SEQUENCE LISTING (35 sequences)

SEQ ID NO: 1 - DNA, 582 bp, Artificial Sequence
  CAG promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcgtcgaggtgagccccacgttctgcttcactctccccatctcccccccctccccacccccaattttgtatttatttattttttaattattttatgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 2 - DNA, 1011 bp, Artificial Sequence
  Chimeric intron
ggagtcgctgcgttgccttcgccccgtgccccgctccgcgccgcctcgcgccgcccgccccggctctgactgaccgcgttactcccacaggtgagcgggcgggacggcccttctccctccgggctgtaattagcgcttggtttaatgacggctcgtttcttttctgtggctgcgtgaaagccttaaagggctccgggagggcctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcgggccgggggcggtgccccgcggtgcgggggggctgcgaggggaacaaaggctgcgtgcggggtgtgtgcgtgggggggtgagcagggggtgtgggcgcggcggtcgggctgtaacccccccctggcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctttagagcctctgctaaccatgttcatgccttcttctttttcctacag

SEQ ID NO: 3 - DNA, 1210 bp, Artificial Sequence
  UCB promoter
ggcctccgcgccgggttttggcgcctcccgcgggcgcccccctcgtcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgagcccttcgctaatgcgggaaagctcttattcgggtgagatgggctgggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagacttgtaaattgtccgctaaattctggccgtttttggcttttttgttagac

SEQ ID NO: 4 - DNA, 390 bp, Artificial Sequence
  PGK promoter
accggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 5 - DNA, 1179 bp, Artificial Sequence
  EF1a promoter plus intron
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 6 - DNA, 939 bp, Artificial Sequence
  EF1a intron
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 7 - DNA, 229 bp, Artificial Sequence
  MECP2 promoter
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 8 - DNA, 1317 bp, Artificial Sequence
  hNSE promoter
atgcagctggacctaggagagaagcaggagaggaagatccagcacaaaaaatccgaagctaaaaacaggacacagagatgggggaagaaaagagggcagagtgaggcaaaaagagactgaagagatgagggtggccgccaggcactttagataggggagaggctttatttacctctgtttgtttttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctgggattacaggggtgagccacagcgcctggtccctatttacttctgtcttctacctccaggagatcaaagacgctggccttcagacctgatcagactcccaggggcagccaccacatgtatgacagagaacagaggatgcctgtttttgccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtcccccacccgcgctcgtacgtgcgcctccgccggcagctcctgactcatcgggggctccgggtcacatgcgcccgcgcggccctataggcgcctcctccgcccgccgcccgggagccgcagccgccgccgccactgccactcccgctctctcagcgccgccgtcgccaccgccaccgccaccgccactaccaccgagatctgcgatctaagtaagcttggcattccggtactgttggtaaagcc

SEQ ID NO: 9 - DNA, 448 bp, Artificial Sequence
  hSyn promoter
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 10 - DNA, 1289 bp, Artificial Sequence
  CamKII promoter
cattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 11 - DNA, 541 bp, Artificial Sequence
  hDLX promoter
ttcagaatgttatgcactcacagtggtttggcatgcatctggtgaattttttttaacgaaaaattagtgttggtttcgatgtatggtagcattctccctaacgtaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtaggaag

SEQ ID NO: 12 - DNA, 49 bp, Artificial Sequence
  pB Globin
ctgggcataaaagtcagggcagagccatctattgcttacatttgcttct

SEQ ID NO: 13 - DNA, 133 bp, Artificial Sequence
  hDLX intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 14 - DNA, 1296 bp, Artificial Sequence
  Endogenous hSLC6A1 promoter
agaatccctcaaacctcaagaactgagagaagggtgtctggggctcctgccaccatccctgtttcccttttaagtaatctgtttccccatctgtccatccatacacacagccacttgtgtctccatgaccaaccgctggcagtggaagggtgtccttcccacccccactcttacacacactcccagctggtacccagagcctggtcaccccaggccaggcctgtgtttccaggtgtaacgggcagcagacgctgccctaggactagagcagggagggggcacgggcccaccccaacccacagcgacccacagagggcgaaaagaggacgaccgcagagagaaacggaaaggacaggccaacggaagcagtactgcaaggctggaaggagaaaagccaggaggggagtgcttgctgtgaaagacagggagacagagaccaagacggacaggcagacaggctggtgacccaggatgaggccggaaagagccatcaaaggaaggagaaggaaggagagagattggagcgggacggcggggcaggcgagggaaggagggggtggggagagggagggaggaagagaggggagaaagagggaggaagagaggggagaaggagggagaagagagcgggagaatgcgagaggaaagaagggagaggggaggcgtagaaggggagaggaggtgaagggaaaaggagagagcctgctggcggcgaagctgcaagaggcagctgcggagggagcgcgcggcgggcctgggggagcgctgggcgggggcgggcggtgcgggcagggctatacccgagctgggcgggctccgggcgccgcgggccctgccctccccctccatccctccggactcgctcccccctcctctcccttccccgcgaccctccgcccgccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgaggcaactccaaggtcctactctctttctgtgcctgttacccaccccgtcctcctagggtgcccttgagccgcaaaactgctgtccacgtggaccgggggtgacatcgcacgtccatctgccaggacccctgcgtccaaattccgagac

SEQ ID NO: 15 - DNA, 1800 bp, Artificial Sequence
  human SLC6A1
atggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctag

SEQ ID NO: 16 - DNA, 1800 bp, Artificial Sequence
  mouse SLC6A1
atggcgactgacaacagcaaggtggctgatgggcagatctctactgaggtcagcgaggcccctgtggccagcgacaagcccaaaaccctggtagtcaaggtgcagaagaaggccggggacctccctgaccgggacacatggaagggacgcttcgacttcctcatgtcctgcgtgggctatgccatcggcctgggcaatgtgtggaggttcccttacctctgtgggaaaaacggtggcggggccttcctaatcccatatttcctgacgctcatctttgcgggtgttcctctcttccttttggagtgctccctaggccagtacacctccattgggggcctgggcgtatggaagctggcgcccatgttcaagggtgtgggcctcgcggcagctgtgctgtccttctggctgaacatctactacatcgtcatcatctcctgggccatctactacctgtacaactccttcaccacgaccctgccatggaaacagtgtgacaacccgtggaacactgaccgctgcttctccaactacagcctggtcaataccaccaacatgaccagcgccgtggtggagttctgggagcgcaacatgcaccagatgacagatggactggacaagccaggacagatccgctggcctctggccatcacactggccattgcctgggtgctcgtgtatttctgcatctggaagggtgttggttggactggaaaggtggtctacttctcagccacgtacccctacatcatgcttatcatcctgttcttccgtggagtgacgcttcccggggccaaggaggggatcctcttctacatcacacccaacttccgaaagctgtctgattctgaggtgtggcttgacgccgccacccagatcttcttctcctacgggctgggcctggggtccctgattgctctgggaagctacaactctttccacaacaatgtgtacagggactccatcatcgtttgctgcatcaactcctgcaccagcatgtttgccggattcgtcatcttctccatcgtgggcttcatggctcatgtcaccaagaggtccatagctgatgtggcagcctcaggcccggggctggcattcttggcgtaccctgaggctgtgacacagctacccatctctcccctctgggctatcctcttcttctccatgctgctgatgctgggcattgacagccagttctgtaccgtggagggcttcatcactgccctggtggacgagtaccccagacttctccgcaatcgccgtgaactcttcattgctgccgtgtgcatcgtgtcctacctgattggcctgtctaacatcacccagggtggcatttatgtcttcaaactgtttgattattactctgccagcggcatgagcttgctgttcctggttttcttcgagtgtgtctccatttcctggttttatggtgtcaaccggttctatgacaacatccaggagatggttggctccaggccctgcatctggtggaagctgtgctggtcctttttcacacccatcattgtggcgggcgtgtttctcttcagtgctgtgcagatgacaccactcaccatgggaagctatgttttccccaagtggggccagggcgtgggctggctcatggctctgtcctccatggtgctcatccccgggtacatggcttacatgttcctcaccctgaagggctccctgaagcagcgtctccaggtcatgattcagcccagtgaagatattgtgcgccctgagaatggccctgagcagccgcaggctggcagctcagccagcaaggaggcctacatctag

SEQ ID NO: 17 - DNA, 135 bp, Artificial Sequence
  SV40 poly A
gatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 18 - protein, 599 aa, Artificial Sequence [sequence omitted]

SEQ ID NO: 19 - protein, 421 aa, Artificial Sequence
  Human GAT-1 isoform b
MVNTTNMTSAVVEFWERNMHQMTDGLDKPGQIRWPLAITLAIAWILVYFCIWKGVGWTGKVVYFSATYPYIMLIILFFRGVTLPGAKEGILFYITPNFRKLSDSEVWLDAATQIFFSYGLGLGSLIALGSYNSFHNNVYRDSIIVCCINSCTSMFAGFVIFSIVGFMAHVTKRSIADVAASGPGLAFLAYPEAVTQLPISPLWAILFFSMLLMLGIDSQFCTVEGFITALVDEYPRLLRNRRELFIAAVCIISYLIGLSNITQGGIYVFKLFDYYSASGMSLLFLVFFECVSISWFYGVNRFYDNIQEMVGSRPCIWWKLCWSFFTPIIVAGVFIFSAVQMTPLTMGNYVFPKWGQGVGWLMALSSMVLIPGYMAYMFLTLKGSLKQRIQVMVQPSEDIVRPENGPEQPQAGSSTSKEAYI

SEQ ID NO: 20 - protein, 421 aa, Artificial Sequence; identical to SEQ ID NO: 19
  Human GAT-1 isoform c

SEQ ID NO: 21 - DNA, 6922 bp, Artificial Sequence
  AAV9 hu14
agaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttcgctgaagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcaggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgcgtcaaaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 22 - DNA, 128 bp, Artificial Sequence
  5′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 23 - DNA, 128 bp, Artificial Sequence
  3′ ITR
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 24 - protein, 735 aa, Artificial Sequence
  AAV true type
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 25 - protein, 736 aa, Artificial Sequence
  AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - DNA, 4417 bp, Artificial Sequence
  Human SLC6A1 transcript variant 2 (isoform a)
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 27 - DNA, 4227 bp, Artificial Sequence
  Human SLC6A1 transcript variant 3 (isoform b)
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 28 - DNA, 4156 bp, Artificial Sequence
  Human SLC6A1 transcript variant 4 (isoform c)
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgaggcaactccaaggtcctactctctttctgtgcctgttacccaccccgtcctcctagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 29 - DNA, 4095 bp, Artificial Sequence
  Human SLC6A1 transcript variant 5 (isoform c)
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 30 - DNA, 6946 bp, Artificial Sequence
  AAV true type DNA sequence
tttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttcgctgaagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagaggatccttgaacctctgggcctggttgaggaacctgttaagacggctccgggaaaaaagaggccggtagagcactctcctgccgagccagactcctcctcgggaaccggaaagagcggccagcagcctgcaagaaaaagattgaattttggtcagactggagacgcagactcagtacctgacccccagcctctcggacagccaccagcagccccctctggtctgggaactaatacgatggctagcggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcagcttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgatgtcaaggcttcagttttctcaggccggagcgagtgacattcgggaccagtctaggaactggcttcctggaccctgttaccgccagcagcgagtatcaaagacagccgcggataacaacaacagtgactactcgtggactggagctaccaagtaccacctcaatggcagagactctctggtgaatccgggcccggccatggcaagccacaaggacgatgaagaaaagtactttcctcagagcggggttctcatctttgggaagcaagactcaggcaaaacaaatgtggacattgaaaaggtcatgattacagacgaagaggaaatcaggacaaccaatcccgtggctacggagcagtatggttctgtatctaccaacctccagagcggcaacacccaagcagctaccagcgatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgcgtcaaaagggcgacacaaaatttattctaaatgcataataaatactgataacatcttatagtttgtattatattttgtattatcgttgacatgtataatttttctagagcggccgcagatctcagctggatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctt

SEQ ID NO: 31 - DNA, 1800 bp, Artificial Sequence; identical to SEQ ID NO: 16
  Mouse SLC6A1

SEQ ID NO: 32 - DNA, 30 bp, Artificial Sequence
  Myc tag
gagcagaaactcatctcagaagaggatctg

SEQ ID NO: 33 - DNA, 27 bp, Artificial Sequence
  HA tag
tacccttacgatgtaccggattacgca

SEQ ID NO: 34 - DNA, 2000 bp, Artificial Sequence
  MECP2 intron
gcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctca

SEQ ID NO: 35 - DNA, 231 bp, Artificial Sequence
  EF1a promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacag

(19) United States
(12) Patent Application Publication
(10) Pub. No.: US 2023/0365652 A1
(43) Pub. Date: Nov. 16, 2023

(54) Nucleic Acid Constructs, Viral Vectors and Viral Particles

(71) Applicant: UCB BIOPHARMA SRL, Brussels (BE)

(72) Inventors: Stefanie Marie Dedeurwaerdere, Slough, Berkshire (GB); Tal Kramer, Slough, Berkshire (GB); Csilla Sipeky, Slough, Berkshire (GB); Brittany Nicole Vallette, Slough, Berkshire (GB); Meiyu Xu, Slough, Berkshire (GB)

(21) Appl. No.: 18/028,736

(22) PCT Filed: Oct. 7, 2021
(86) PCT No.: PCT/EP2021/077666

§ 371 Date: Mar. 27, 2023

Related U.S. Application Data
(60) Provisional application No. 63/089,817, filed on Oct. 9, 2020.

(51) Int. Cl. C07K 14/705; C12N 15/86; A61P 43/00

(52) U.S. Cl. CPC C07K 14/70571; C12N 15/86; A61P 43/00; C12N 2750/14143

(57) ABSTRACT

The present invention relates to nucleic acid constructs, viral vectors and viral particles comprising a transgene encoding GAT-1; and use of such viral particles for treating diseases mediated by SLC6A1-impairment.

### FIELD OF THE INVENTION

The present invention belongs to the field of nucleic acid constructs, viral vectors and viral particles for use in the treatment and/or prevention of disease associated with a loss of solute carrier family 6 member 1 (SLC6A1) function such as myoclonic atonic epilepsy (MAE), MAE-like and other epilepsy indications such as Lennox-Gastaut Syndrome as well as autism spectrum disorder and schizophrenia.

### BACKGROUND OF THE INVENTION

To date, thousands of genes have been associated with neurodevelopmental disorders and with the aid of clinical genetic testing, syndromes are increasingly defined by the mutated gene rather than their clinical characteristics. Disruption of the gene SLC6A1 has been identified as a prominent cause of a wide range of neurodevelopmental disorders, including autism spectrum disorder (ASD), intellectual disability (ID), and seizures of varying types and severity. SLC6A1 encodes GAT-1, a member of the gamma-amino butyric acid (GABA) transporter family expressed in the central nervous system (Bröer S. and Gether U. 2012. Br J Pharmacol 167: 256-278). The SLC6A1 gene was first cloned in 1990 (Guastella J. et al. 1990. Science 249: 1303-1306) and belongs to a family of 20 paralogs. The proteins encoded by 13 of these genes exhibit above 80% sequence identity and six of them are able to transport GABA with different degrees of substrate specificity.

GAT-1 is expressed broadly and exclusively in the mammalian central nervous system, predominantly in the frontal cortex in the adult human brain (Gamazon E. R. et al. 2018. Nat Genet 50: 956-967). Unlike other GABA transporters, GAT-1 is almost exclusively expressed in GABAergic axon terminals and astrocytes. In the developing brain, GABA exerts an excitatory action, but later becomes the main inhibitory neurotransmitter in the central nervous system. The onset of GABAergic inhibition is important to counterbalance neuronal excitation, and when significantly disrupted, it negatively impacts brain development leading to attention and cognitive deficits as well as seizures.

The GAT-1 protein is composed by 12 transmembrane domains that come together to form a single chain transporter. The primary function of GABA transporters is to lower the concentration of GABA in the extracellular space (Scimemi A. 2014. Front Cell Neurosci 8). This task is accomplished by coupling the translocation of GABA across the cell membrane with the dissipation of the electrochemical gradient for sodium and chloride (FIG. 1). By moving these ions across the membrane in fixed ratio with GABA (1 GABA:2 Na⁺:1 Cl⁻), GAT-1 generates a stoichiometric current (Lester H. A. et al. 1994. Annual Review of Pharmacology and Toxicology 34: 219-249). At rest, in the pre-synaptic terminal of GABAergic neurons, the driving force for sodium and chloride forces these ions to move from the extracellular space towards the cell cytoplasm, thus carrying GABA in the same direction. The translocation of GABA across the membrane is relatively rapid, allowing GABA to be removed from the extracellular space within few milliseconds after its release (Isaacson et al. 1993. Neuron 10: 165-175). In addition to regulating the transport of GABA, GAT-1 also behaves as an ion channel, and generates two ionic currents that are not stoichiometrically coupled to the movement of GABA across the membrane. The first is a sodium inward current activated by GABA binding to GAT-1 (Risso et al. 1996. J Physiol 490: 691-702). The second is a leak current that can be detected even in the absence of GABA and is mediated, in vitro, by alkali ions like lithium and caesium (MacAulay et al. 2002. J Physiol (Lond) 544: 447-458). Last, in the absence of GABA, GAT-1 generates sodium-dependent capacitive currents (Mager et al. 1993. Neuron 10: 177-188). Through the coordinated activation of these currents, GAT-1 activation can generate a local shunt (i.e. a change in membrane resistance) or membrane depolarization.

There are five major splice variants of human SLC6A1 encoding three GAT-1 isoforms that differ from one another for alternative use of exons three to five. The transcript ENST00000287766 is the longest isoform of SLC6A1 and is considered canonical (Hunt et al. 2018. Database (Oxford) 2018 https://academic.oup.com/database/article/doi/10.1093/database/bay119/5255129). Thus, most genetic variants are mapped into its sequence. The exact topology of GAT-1 remains unclear due to lack of a crystal structure. Homology modeling of GAT-1 (based on the crystal structure of LeuTAa, a prokaryotic homolog leucine transporter from Aquifex aeolicus with 20-25% sequence homology to GAT-1) allowed the identification of residues that are essential for substrate and sodium binding in transmembrane domains 1,3,6,8 and others necessary for the conformational transitions during the transport process (Bröer S. and Gether U. 2012. Br J Pharmacol 167: 256-278).

However, as in the case of many other neurodevelopmental disorder-associated genes, patient variants within SLC6A1 are broadly distributed along its sequence (Johannesen et al. 2018. Epilepsia 59: 389-402). Two types of variants have been observed in patients: (i) protein truncating variants that stop the protein production for one of the two SLC6A1 gene alleles inherited and (ii) missense variants in critical regions of the protein such as GABA binding sites and transmembrane domains.

Thus, the expected molecular pathological mechanism of SLC6A1 disorders is a loss of function or haploinsufficiency. The disease-model is supported by experiments in both wild type and GAT-1−/− mice, as well as studies on recombinant GAT-1 proteins from individuals with SLC6A1 mutations. However, the mechanisms by which the haploinsufficiency lead to the clinical manifestations are not well understood. Recently, experimental evidence showed that SLC6A1 variants identified in epilepsy patients reduce GABA transport in vitro (Mattison et al. 2018; Cai et al. 2019. Epilepsia 59: e135-e141). Other evidence suggests that SLC6A1 mutations may also cause impaired protein trafficking (Cai et al. 2019. Experimental Neurology 320: 112973).

Currently there is no specific animal model of SLC6A1 genetic disorder. Heterozygous (Het) GAT-1 knockout mice appear phenotypically normal despite having greatly diminished GABA reuptake capacity. Functional GAT-1 KO mice have been previously developed and partially characterized (Chiu et al. 2005. Neurosci 25: 3234-3245; Cope et al. 2009. Nature Medicine 15: 1392-1398; Jensen et al. 2003. Neurophysiology 90: 2690-2701; Lester et al. 1994. Annual Review of Pharmacology and Toxicology 34: 219-249). The full KO animals exhibit absence seizures, a constant tremor, abnormal gait, reduced strength and mobility, as well as anxious behaviours (Chiu et al. 2005. Neurosci 25: 3234-3245; Cope et al. 2009. Nature Medicine 15: 1392-1398). These phenotypes match some of the clinical manifestations of SLC6A1 disorder, which include absence seizures, mobility and cognitive impairment (Johannesen et al. 2018. Epilepsia 59: 389-402).

Valproic acid by itself or in combination with other antiepileptic drugs such as vigabatrine has shown positive results (Johannesen et al. 2018. Epilepsia 59: 389-402). Small molecule or chaperone therapies have also been considered theoretically plausible options to enhance activity of the existing GAT-1 proteins but none has been successful so far. None of these intervention address all, or even a small part, of the pathological traits underlying the very diverse clinical manifestations associated with GAT-1 impairment. Hence, there is still a clear unmet medical need for improved treatment options for SLC6A1-associated disorders.

### SUMMARY OF THE INVENTION

The present invention addresses the above-identified need by providing by mean of gene therapy a healthy copy of the wild type SLC6A1 gene that may be subject to endogenous regulatory mechanisms in the transduced cell and capable of restoring GAT-1 transporter function to the ‘normal’ range.

The present invention may be summarised as follows:

Embodiment 1: A nucleic acid construct comprising a transgene encoding:
i. a gamma butyric acid (GABA) transporter protein 1 (GAT-1) comprising SEQ ID NO: 18, 19, 20; or
ii. a sequence having at least 95%, or 96% or 97% or 98% or 99% or 99.5% sequence identity to SEQ ID NO: 18, 19 or 20 and retaining functionality as GAT-1; or
iii. a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile.

Embodiment 2: The nucleic acid construct according to Embodiment 1 wherein the transgene is a solute carrier family 6 member 1 (SLC6A1) gene, wherein the transgene preferably comprises:
i. SEQ ID NO: 15, 26, 27, 28 or 29, preferably SEQ ID NO: 15 or
ii. a sequence having at least 95%, or 96% or 97% or 98% or 99% or 99.5% sequence identity to SEQ ID NO: 15, 26, 27, 28 or 29.

Embodiment 3: The nucleic acid construct according to any one of Embodiments 1 or 2, further comprising a promoter operably linked to said transgene, wherein said promoter preferably comprises:
a. SEQ ID NO: 1, or preferably SEQ ID NO: 1 operably linked in a 5′ to 3′ orientation to SEQ ID NO: 2; or
b. SEQ ID NO: 3; or
c. SEQ ID NO: 4; or
d. SEQ ID NO: 5 or SEQ ID NO: 35 or SEQ ID NO: 6, or preferably SEQ ID NO: 35 operably linked in a 5′ to 3′ orientation to SEQ ID NO: 6; or
e. SEQ ID NO: 7; or preferably SEQ ID NO: 7 operably linked in a 5′ to 3′ orientation to SEQ ID NO: 34; or
f. SEQ ID NO: 8; or
g. SEQ ID NO: 9; or
h. SEQ ID NO: 10; or
i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
j. SEQ ID NO: 14.

Embodiment 4: The nucleic acid construct according to any one of the preceding Embodiments, wherein the construct comprises a polyadenylation signal sequence, preferably a polyadenylation signal sequence comprising SEQ ID NO: 17.

Embodiment 5: A viral vector comprising the nucleic acid construct according to any one of the preceding Embodiments, wherein the viral vector further comprises inverted terminal repeat (ITR) at 5′ and/or 3′ of said nucleic acid construct, preferably 5′ITR and 3′ITR.

Embodiment 6: The viral vector according to Embodiment 5, wherein the 5′ITR and/or the 3′ITR comprises the ITR of a natural adeno-associated virus (AAV), such as AAV2.

Embodiment 7: The viral vector according to any one of Embodiments 5 or 6, wherein the 5′ITR comprises SEQ ID NO: 22 and/or the 3′ITR comprises SEQ ID NO: 23.

Embodiment 8: A viral particle comprising a nucleic acid construct according to any one of Embodiments 1 to 4 or a viral vector according to any one of Embodiments 5 to 7.

Embodiment 9: The viral particle according to Embodiment 8, wherein the viral particle comprises at least a VP1 capsid protein from an AAV, wherein said capsid protein preferably comprises AAV2, AAV5, AAV6, AAV8, AAV9 (such as comprising SEQ ID NO: 25), AAV10, AAV-true type (AAVtt such as comprising SEQ ID NO: 24) or combinations thereof.

Embodiment 10: The viral particle according to Embodiment 9, wherein the capsid protein is from AAVtt and preferably comprises SEQ ID NO: 24 or it is at least 98.5%, preferably 99% or 99.5% identical to SEQ ID NO: 24.

Embodiment 11: A viral vector comprising a nucleic acid construct comprising a transgene encoding:
i. a gamma butyric acid (GABA) transporter protein 1 (GAT-1) comprising SEQ ID NO: 18, 19, 20; or
ii. a sequence having at least 95% or 96% or 97% or 98% or 99% or 99.5% sequence identity to SEQ ID NO: 18, 19 or 20 and retaining functionality as GAT-1; or
iii. a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Vail 42Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
   wherein said viral vector further comprises a promoter operably-linked to said transgene, wherein said promoter preferably comprises SEQ ID NO: 4 or SEQ ID NO: 14; wherein the nucleic acid construct comprised in said viral vector comprises a polyadenylation signal sequence, preferably a polyadenylation signal sequence comprising SEQ ID NO: 17; and wherein said viral vector further comprises inverted terminal repeat (ITR) at 5′ and/or 3′ flanking said nucleic acid construct, preferably a 5′ITR and a 3′ITR.

Embodiment 12. A viral vector comprising a nucleic acid construct comprising a transgene which is a solute carrier family 6 member 1 (SLC6A1) gene, wherein the transgene preferably comprises:
i. SEQ ID NO: 15, 26, 27, 28 or 29, more preferably SEQ ID NO: 15;
ii. or a sequence having at least 95% or 96% or 97% or 98% or 99% or 99.5% sequence identity to SEQ ID NO: 15, 26, 27, 28 or 29.
   wherein said viral vector further comprises a promoter operably-linked to said transgene, wherein said promoter preferably comprises SEQ ID NO: 4 or SEQ ID NO: 14; wherein the nucleic acid construct comprised in said viral vector comprises a polyadenylation signal sequence, preferably a polyadenylation signal sequence comprising SEQ ID NO: 17 and wherein said viral vector further comprises inverted terminal repeat (ITR) at 5′ and/or 3′ flanking said nucleic acid construct, preferably a 5′ITR and a 3′ITR.

Embodiment 13. The viral vector according to any one of Embodiments 11 or 12, wherein said transgene encodes a gamma butyric acid (GABA) transporter protein 1 (GAT-1) comprising SEQ ID NO: 18.

Embodiment 14. The viral vector according to any one of Embodiments 11 to 13, wherein the polyadenylation signal sequence comprises SEQ ID NO: 17.

Embodiment 15: A viral particle comprising the viral vector according to any one of Embodiments 11 to 14.

Embodiment 16: The viral particle according to Embodiment 15, wherein the viral particle comprises at least a VP1 capsid protein from an AAV, wherein said capsid protein preferably comprises AAV2, AAV5, AAV6, AAV8, AAV9 (such as comprising SEQ ID NO: 25), AAV10, AAV-true type (AAVtt) or combinations thereof.

Embodiment 17: The viral particle according to Embodiment 16, wherein the capsid protein is from AAV9 and preferably comprising SEQ ID NO: 25 or AAVtt and preferably comprises SEQ ID NO: 24 or it is at least 98.5%, preferably 99% or 99.5% identical to SEQ ID NO: 24.

Embodiment 18: A plasmid comprising the nucleic acid construct according to any one of Embodiments 1 to 4 or the viral vector according to any one of Embodiments 5 to 7 or 11 to 14.

Embodiment 19: A host cell for producing a viral particle according to any one of Embodiments 8 to 10 or 15 to 17.

Embodiment 20: The host cell according to Embodiment 18, wherein the host cell comprises:
a. a nucleic acid construct according to any one of Embodiments 1 to 4 or the viral vector according to any one of Embodiments 5 to 7 or 11 to 14;
b. a nucleic acid construct, preferably a plasmid, encoding AAV rep and/or cap genes which does not carry the ITR sequences; and, optionally
c. a nucleic acid construct, for example a plasmid or virus, comprising viral helper genes.

Embodiment 21: A method of producing a viral particle according to any one of Embodiments 8 to 10 or 15 to 17, the method comprising the step of:
a. culturing a host cell according to any one of Embodiments 19 or 20 in a culture medium; and
b. harvesting the viral particles from the host cell culture media and/or inside the host cells.

Embodiment 22: A pharmaceutical composition comprising a nucleic acid construct according to any one of Embodiments 1 to 4 or the viral vector according to any one of Embodiments 5 to 7 or 11 to 14, or a viral particle according to any one of Embodiments 8 to 10 or 15 to 17, in combination with one or more pharmaceutical acceptable excipient, diluent or carrier.

Embodiment 23: The viral particles according to any one of Embodiments 8 to 10 or 15 to 17 for use in therapy.

Embodiment 24: The viral particles for use according to any one of Embodiments 8 to 10 or 15 to 17 in the treatment and/or prevention of disease characterised by SLC6A1 haploinsufficiency, wherein the disease preferably comprises single-gene epilepsies accompanied by cognitive, motor behavioural comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof.

Embodiment 25: The viral particle for use according to any one of Embodiments 23 or 24, wherein the use is for restoring GAT-1 function and/or decreasing seizure frequency.

Embodiment 26: The viral particle for use according to any one of Embodiments 8 to 10 or 15 to 17, wherein said disease is associated with at least one mutation in a patient which leads to a pathological GAT-1 variant, wherein said pathological GAT-1 variants comprises a mutation or combinations of mutations.

Embodiment 27: The viral particle for use according to Embodiment 26, wherein said mutation comprises, with reference to SEQ ID NO: 18, R44W, R44Q, R50L, D52E, D52V, F53S, S56F, G63S, N66D, G75R, G79R, G79V, F92S, G94E, G105S, Q106R, G112V, Y140C, 0173Y, G232V, F270S, R277H, A288V, S295L, G297R, A305T, G307R, V323I, A334P, V342M, A357V, G362R, L366V, A367T, F385L, G393S, S456R, S459R, M487T, V511L, G550R or combination thereof.

Embodiment 28: A method of treating and/or preventing a disease characterised by SLC6A1 haploinsufficiency, wherein the disease preferably comprises single-gene epilepsies accompanied by cognitive, motor behavioural comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof, the method comprising administering to a subject in need thereof of viral particles according to any one of embodiments 8 to 10 or 14 to 16.

Embodiment 29: The method according to Embodiment 28, wherein the method is for restoring GAT-1 function and/or decreasing seizure frequency.

Embodiment 30: The method according to any one of Embodiments 28 or 29, wherein said disease is associated with at least one mutation in a patient which leads to a pathological GAT-1 variant, wherein said pathological GAT-1 variants comprises a mutation or combinations of mutations.

Embodiment 31: The method according to Embodiment 30 wherein said mutation comprises, with reference to SEQ ID NO: 18, R44W, R44Q, R50L, D52E, D52V, F53S, S56F, G63S, N66D, G75R, G79R, G79V, F92S, G94E, G105S, Q106R, G112V, Y140C, 0173Y, G232V, F270S, R277H, A288V, S295L, G297R, A305T, G307R, V323I, A334P, V342M, A357V, G362R, L366V, A367T, F385L, G393S, S456R, S459R, M487T, V511L, G550R or combination thereof.

### BRIEF DESCRIPTION OF THE DRAWINGS

FIG. 1. Cartoon illustrating the SLC6A1 encoded GAT-1 transporter and its function. GAT-1 is a solute carrier protein which regulates the uptake of extracellular GABA. Stoichiometry of GAT-1: one molecule of inhibitory neurotransmitter GABA is co-transported together with two sodium cations and one chloride anion along the electrochemical gradient.

FIG. 2. Protein sequence alignment of the human, monkey and mouse GAT-1 sequences (human variant according to SEQ ID NO: 18). The alignment shows the high sequence identity across the three species.

FIG. 3. Schematic cartoon of the designed constructs. In the figure, “prom”=promoter in general and the various promoters analysed are illustrated at the bottom (CAG, EF1a, PGK and UcB); “INT” means intron and “EX” means exon, “h” or “m”=human and mouse, respectively, SV40 means polyadenylation sequence SV40; “tag”=an HA or myc tag, located either at the N or at the C terminus of a construct with the CAG promoter.

FIG. 4. AD-HEK293 cells transfected with hSLC6A1 and mSLC6A1 plasmids driven by different ubiquitous promoters. The magnification section shows that GAT-1 was transported to the expected cellular localization.

FIG. 5. A: Neuro-2A cells transfected with mSLC6A1 plasmids driven by different neuron-specific promoters. B: Magnification showing that GAT-1 was transported to the expected cellular localization.

FIG. 6. Western blot analysis of (A) HA- and (B) Myc-tagged mSLC6A1 and hSLC6A1 in AD-HEK293 cells. Two technical replicates of each condition are shown. (C) Epitope tagged proteins were also detected using anti-SLC6A1 antibodies. C=Control, 1=CAG-HA-hSLC6A1, 2=CAG-hSLC6A1-Myc, 3=CAG-Myc-hSLC6A1, 4=CAG-Myc-mSLC6A1, 5=CAG-mSLC6A1-Myc, H=human brain lysate, M=mouse brain lysate.

FIG. 7. (A) Schematic cartoon of the designed constructs. In the figure, “h”=human, WT=wild type, p.=protein, IRES Internal ribosome Entry Site, tagRFP=tag red fluorescent protein, SV40=polyadenylation sequence from simian virus 40; (B) Tritiated [³H] GABA uptake assay in transfected COS-7 cells. Results are shown as Mean+SD and normalized to the CAG-hSLC6A1-WT-IRES-tag RFP construct.

FIG. 8. Tritiated [³H] GABA uptake assay in transfected SHSY-5Y cells. Cells were transfected with plasmid containing AAV ITRs (pAAV) where hSLC6A1 expression is driven by the different promoters. Results are shown as Mean+SD and normalized to the CAG-hSLC6A1-WT-IRES-tag RFP construct.

FIG. 9. Lentivirus transduction in iPSCs derived NGN2 neurons. One representative picture is shown per condition with only the channel used to visualise GAT-1.

FIG. 10. (A) Absolute quantification by qPCR of viral genome copies using SV40pA (polyA signal of simian virus 40) normalized to the absolute number of diploid mouse genome. Results are shown as median+interquartile range. (B) RNA expression analysis. Data are shown as relative expression that were scaled to the average expression of all groups. Results are shown as geometric mean+geometric SD

FIG. 11. Protein analysis by Western blot of samples from the right frontal cortex. Panels A, C and E: Western blot representing the GAT-1 expression in the different constructs tested (n=5). Mice from the “control AAV9” group and the vehicle-PBS control group are the same in all three panels. Panels B, D, and F are quantification data of the respective Western blots, GAPDH was used as loading control and for normalization of each GAT-1 band intensity. Results are shown as Mean+SD. The “control AAV9” group was used as the scaling group. Panel G: Western blot representing the HA and GAPDH expression (loading control) of the 3 constructs put together. Panel H: The Western blot represented in Panel G was reproduced twice and the data were quantified, averaged for each sample and shown here. Results are shown as Mean+SD.

FIG. 12. Triple immunolabeling for GFAP (astrocytes), NeuN (neurons) and HA (human GAT-1) in sagittal sections from the mouse brain. AF=Alexa Fluor.

FIG. 13. Triple immunolabeling for GFAP (astrocytes), NeuN (neurons) and HA (human GAT-1) in sagittal sections from the mouse hippocampus. AF=Alexa Fluor.

FIG. 14. Triple immunolabeling for GFAP (astrocytes), NeuN (neurons) and HA (human GAT-1) in sagittal sections from the mouse cerebral cortex. AF=Alexa Fluor.

FIG. 15. Average number of SWDs in SLC6A1^{+/S295L} mice injected with vehicle-PBS (n=11), AAV9-PGK-HA-hSCL6A1 (n=8), AAV9-ENDO-HA-hSCL6A1 (n=13) and AAV9-hDLX-HA-hSCL6A1 (n=9). SWDs were analyzed 6 weeks after injection over a period of 5 hours between 1 μm and 6 pm for 7 consecutive days. The difference between groups was analyzed by non-parametric one-way ANOVA (Kruskal-Wallis test) followed by a Dunn's post hoc multiple comparisons test (**p<0.01; ***p<0.001; ns, nonsignificant).

FIG. 16. (A) Absolute quantification by qPCR of viral genome copies using SV40pA (polyA signal of simian virus 40) normalized to the absolute number of diploid mouse genome (ValidPRime®). Results are shown as Mean+SD. The difference between groups (n=10-15) was analyzed by non-parametric one-way ANOVA (Kruskal-Wallis test) followed by a Dunn's post hoc multiple comparisons test. No significant difference was observed between the groups. (B) RNA expression analysis of human SLC6A1. Data are shown as relative expression that were scaled to the average expression of all groups. Results are shown as geometric mean+geometric SD. The difference between groups (n=10-15) was analyzed by non-parametric one-way ANOVA (Kruskal-Wallis test) followed by a Dunn's post hoc multiple comparisons test (**p<0.01; ***p<0.001; ns, nonsignificant).

FIG. 17. Protein analysis by Western blot of samples from the half medial frontal cortex. Panels A, B and C: Western blots representing the GAT-1 (SLC6A1) protein expression from the different viral vectors studied, PBS control group and WT (wild-type) group (n=7-10). Mice from the WT (wild-type) group and the HET (SLC6A1^{+/S295L} mice) group are the same in all three panels. Panels D, E, and F are quantification data of the respective Western blots, GAPDH was used as loading control and for normalization of each GAT-1 band intensity. Results are shown as Mean+SD. The WT group was used as the scaling group. Panel G and H: Western blots representing the HA and GAPDH expression (loading control) of the 3 viral vectors put together. Panel I: Combined quantification of the Western blot represented in Panel G and H. GAPDH was used as loading control and for normalization of each GAT-1 band intensity. Results are shown as Mean+SD. The PGK group was used as the scaling group for comparison of the promoters. The data was analyzed using one-way ANOVA followed by a Tukey's multiple comparisons test (* p<0.01 **p<0.001, ***p<0.0001).

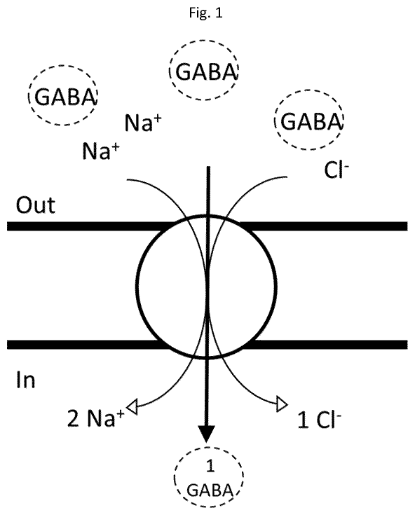

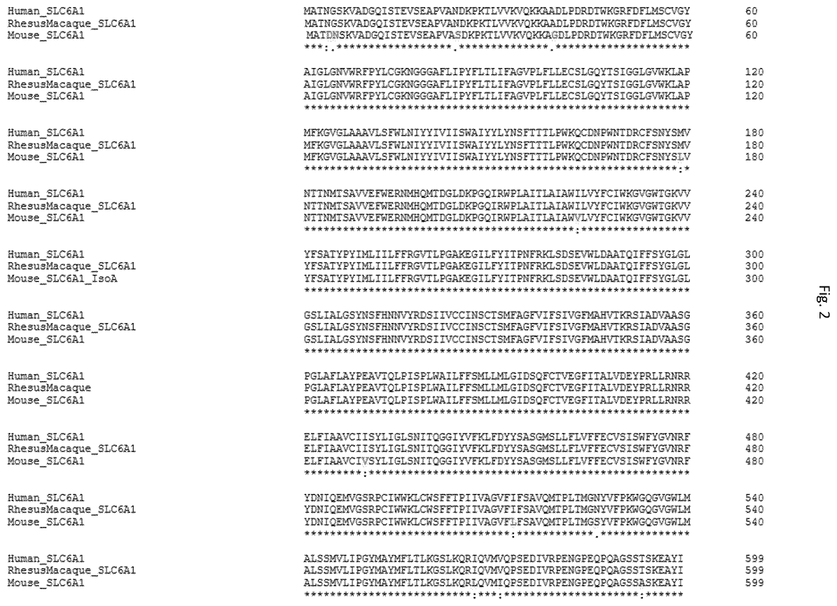

### DETAILED DESCRIPTION OF THE INVENTION

The present invention will now be described with respect to particular non-limiting aspects and embodiments thereof and with reference to certain figures and examples.

Technical terms are used by their common sense unless indicated otherwise. If a specific meaning is conveyed to certain terms, definitions of terms will be given in the context of which the terms are used.

Where an indefinite or definite article is used when referring to a singular noun, e.g. “a”, “an” or “the”, this includes a plural of that noun unless something else is specifically stated.

As used here, the term “comprising” does not exclude other elements. For the purposes of the present disclosure, the term “consisting of” is considered to be a preferred embodiment of the term “comprising of”.

As used herein, the terms “treatment”, “treating” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Treatment thus covers any treatment of a disease in a mammal, particularly in a human, and includes: (a) preventing the disease from occurring in a subject, i.e. a human, which may be predisposed to the disease but has not yet been diagnosed as having it; (b) inhibiting the disease, i.e., arresting its development; and (c) relieving the disease, i.e., causing regression of the disease.

The present invention provides for a nucleic acid construct comprising a transgene encoding a gamma butyric acid (GABA) transporter protein 1 (GAT-1) comprising SEQ ID NO: 18, 19, 20 or a sequence having at least 95% sequence identity to SEQ ID NO: 18, 19, 20 and retaining functionality as GAT-1.

As used herein, the term “transgene” refers to nucleic acid molecule (or nucleic acid in short and interchangeably used herein), DNA or cDNA encoding a gene product for use as the active principle in gene therapy. The gene product may be one or more peptides or proteins.

In one embodiment, the transgene is a solute carrier family 6 member 1 (SLC6A1) gene.

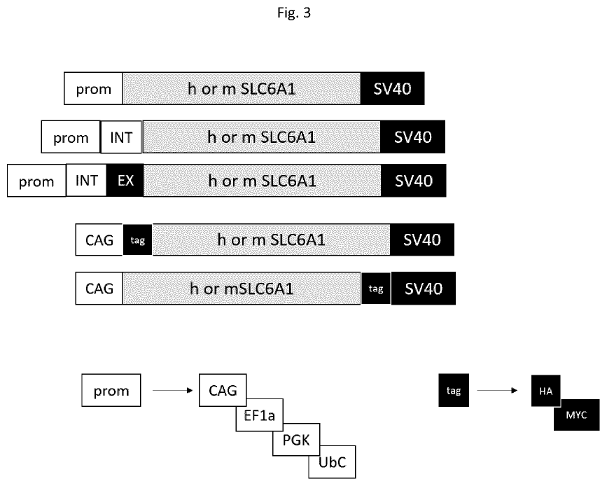

The SLC6A1 gene is located in the short arm of chromosome 3 (GRCh38 genomic coordinates: 3:10,992,733-11,039,248 10,992,748-11,039,247) between the SLC6A11 gene (encoding another type of GABA transporter) and the HRH1 gene (encoding the histamine receptor H1). The SLC6A1 gene is approximately 46.5 Kilobase (Kb) long and comprises 18 exons (https://www.ncbi.nlm.nih.gov/gene/6529). There are five major variants leading to 3 splice isoforms (a, b and c) of human GAT-1 that differ from one another for alternative use of exons three to five. The transcript ENST00000287766 corresponding to the coding sequence portion CDS is the longest isoform of human SLC6A1 and is considered canonical (Hunt et al. 2018) (FIG. 2) and comprises SEQ ID NO: 15. Thus, most genetic variants are mapped into this sequence. Known genetic variants comprise variants 2 comprising SEQ ID NO: 26, variant 3 comprising SEQ ID NO: 27, variant 4 comprising SEQ ID NO: 28 and variant 5 comprising SEQ ID NO: 29.

In particular, the nucleic acid construct according to the present invention comprises a transgene encoding GAT-1, preferably encoding human GAT-1, wherein the transgene comprises SEQ ID NO: 15, 26, 27, 28 or 29, more preferably SEQ ID NO: 15.

As used herein, the term “GAT-1” refers to gamma butyric acid (GABA) transporter protein 1 (GAT-1) (also called GABA transporter 1; MAE; GAT1; GABATR; GABATHG (Uniprot code: P30531). GAT-1 protein is composed by 12 transmembrane domains that come together to form a single chain transporter. The five splice variants of human SLC6A1 leads to three splice isoforms of GAT-1, isoform a comprising SEQ ID NO: 18 (which is considered the canonical sequence), encoded by splice variants 1 or 2, comprising SEQ ID NO: 15 and 26, respectively; isoform b, comprising SEQ ID NO: 19, encoded by splice variant 3 comprising SEQ ID NO: 27; and isoform c, comprising SEQ ID NO: 20, encoded by splice variants 4 or 5, comprising SEQ ID NO: 28 and 29, respectively. As used herein, the term GAT-1 refers to all variants and isoforms of GAT-1 described herein (unless specified otherwise).

Hence, in one embodiment, the nucleic acid construct comprises a transgene encoding a gamma butyric acid (GABA) transporter protein 1 (GAT-1) comprising:
i. SEQ ID NO: 18, 19, 20; or
ii. a sequence having at least 95% or 96% or 97% or 98% or 99% or 99.5% sequence identity to SEQ ID NO: 18 or 19 or 20 and retaining functionality as GAT-1; or
iii. a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; IIe84Phe; Ala221Thr; Ile405Val; Thr558Asn; Phe87Leu; Val240Ala; Val409Met; Arg566His; Ile91Val; Phe242Val; Leu415Ile; Gln572Arg; Vail 42Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
   wherein the transgene is a solute carrier family 6 member 1 (SLC6A1) gene, preferably comprising SEQ ID NO: 15, 26, 27, 28 or 29, more preferably SEQ ID NO: 15; or a or a sequence having at least 95% or 96% or 97% or 98% or 99% or 99.5% sequence identity to SEQ ID NO: 15, 26, 27, 28 or 29.

The terms “nucleic acid” and “polynucleotide” or “nucleotide sequence” may be used interchangeably to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholinos and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2′-O-allyl analogs and 2′-O-methylribonucleotide methylphosphonates which may be used in a nucleotide of the invention.

Furthermore, the term “nucleic acid construct” refers to a non-naturally occurring nucleic acid resulting from the use of recombinant DNA technology. Especially, a nucleic acid construct is a nucleic acid molecule which has been modified to contain segments of nucleic acid sequences, which are combined or juxtaposed in a manner which would not otherwise exist in nature.

In specific embodiments, said nucleic acid construct comprises all or a fragment (at least 1000, 1100, 1500, 2000, 2500 or at least 1500 nucleotides) of a coding nucleic acid sequence having at least 70%, 80%, 90%; 95%, 99% or 100% identity to the coding sequence of a naturally-occurring or recombinant functional variant of GAT-1. Naturally occurring GAT-1 variants include human, primate, murine or other mammalian known GAT-1, typically human GAT-1 comprising SEQ ID NO: 18, 19 or 20.

The term “fragment” as used herein refers to a contiguous portion of a reference sequence. For example, a fragment of SEQ ID NO: 18 or 19 or 20 of at least 1000 nucleotides in length refers to 50, or 100 or 200 or 500 or 1000 and so for contiguous nucleotides of SEQ ID NO: 18 or 19 or 20.

The term “functional variant” or “a naturally-occurring variant” as used herein refers to a nucleic acid or amino acid sequence which has been modified relative to a reference sequence but which retains the function of said reference sequence. For example, a functional variant of SLC6A1 retains the ability to encode a GAT-1. Similarly, a functional variant of a GAT-1 retains the activities of the reference GAT-1. Naturally-occurring variants of GAT-1, with reference to SEQ ID NO: 18, are shown in Table 3 and comprise, with reference to SEQ ID NO: 18, one or more mutations preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Vail 42Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile.

In a preferred embodiment, said nucleic acid construct comprises a transgene encoding human GAT-1, wherein said human GAT-1 comprises SEQ ID NO: 18 or 19 or 20 for example, a transgene comprising a SEQ ID NO: 15, or a variant of said transgene consisting of a nucleotide sequence having at least 75%, at least 80% or at least 90%, at least 95% or at least 99% identity to SEQ ID NO: 15. In one embodiment, the variant of said transgene comprises i) a nucleotide sequence encoding a portion of GAT-1 comprising SEQ ID NO: 18 or 19 or 20 or ii) a nucleotide sequence having at least 75%, at least 80% or at least 90%, at least 95% or at least 99% identity to SEQ ID NO: 15 and retaining substantially the same GAT-1 activity as human GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417H is; Pro573Ser; Thr158Pro; Arg257His; Arg419Cys; Ser574Asn; Asp165Asn; Thr260Met; Arg419His; or Val578Ile.

As used herein, the term “sequence identity” or “identity” refers to the number of matches (identical nucleic acid or amino acid residues) in positions from an alignment of two polynucleotide or polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970, J Mol Biol.; 48(3):443-53) which aligns the sequences optimally over the entire length, while sequences of substantially different lengths are preferably aligned using a local alignment algorithm (e.g. Smith and Waterman algorithm (Smith and Waterman, 1981, J Theor Biol.; 91(2):379-80) or Altschul algorithm (Altschul S F et al., 1997, Nucleic Acids Res.; 25(17):3389-402.; Altschul S F et al., 2005, Bioinformatics.; 21(8):1451-6). Alignment for purposes of determining percent nucleic acid or amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/. Those skilled in the art can determine appropriate parameters for measuring alignment, including any algorithms needed to achieve maximal alignment over the full length of the sequences being compared. For purposes herein, % nucleic acid or amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

The nucleic acid construct according to the present invention comprises a transgene and at least a suitable nucleic acid element for its expression for example in a host, such as in a host cell.

For example, said nucleic acid construct comprises a transgene encoding GAT-1 and one or more control sequences required for expression of GAT-1 in the relevant host. Generally, the nucleic acid construct comprises a transgene (such as the one encoding GAT-1) and regulatory sequences preceding (5′ non-coding sequences) and following (3′ non-coding sequences) the transgene that are required for expression of GAT-1.

Thus, in specific embodiments, said nucleic acid construct comprises at least (i) a transgene encoding GAT-1 and ii) a promoter operably linked to said transgene. Preferably, the transgene is under the control of the promoter.

As used herein, the term “promoter” refers to a regulatory element that directs the transcription of a nucleic acid to which it is operably linked. A promoter can regulate both rate and efficiency of transcription of an operably-linked nucleic acid. A promoter may also be operably-linked to other regulatory elements which enhance (“enhancers”) or repress (“repressors”) promoter-dependent transcription of a nucleic acid. These regulatory elements include, without limitation, transcription factor binding sites, repressor and activator protein binding sites, and any other sequences of nucleotides known to one of skill in the art to act directly or indirectly to regulate the amount of transcription from the promoter, including e.g. attenuators, enhancers, and silencers. The promoter is located near the transcription start site of the gene or coding sequence to which is operably linked, on the same strand and upstream of the DNA sequence (towards the 5′ region of the sense strand). A promoter can be about 100-1000 base pairs long. Positions in a promoter are designated relative to the transcriptional start site fora particular gene (i.e., positions upstream are negative numbers counting back from −1, for example −100 is a position 100 base pairs upstream).

As used herein the term “operably linked in a 5′ to 3′ orientation” or simply “operably linked” refer to a linkage of two or more nucleotide sequences in a functional relationship which allows each of said two or more sequences to perform their normal function. Typically, the term operably-linked is used to refer to the juxtaposition of a regulatory element such as promoter and a transgene encoding a protein of interest. For example, an operable linkage between a promoter and a transgene permits the promoter to function to drive the 5′ expression of the transgene in a suitable expression system, such as in a cell.

Typically, such promoter may be tissue or cell type specific promoter, or an organ-specific promoter, or a promoter specific to multiple organs or a systemic or ubiquitous promoter.

As used herein, the term “ubiquitous promoter” more specifically relates to a promoter that is active in a variety of distinct cells or tissues, for example in both the neurons and astrocytes.

Examples of promoter suitable for expression of the transgene across the central nervous system include chicken beta actin (CBA) promoter (Miyazaki 1989, Gene 79:269-277), the CAG promoter (Niwa 1991, Gene 108:193-199), the Elongation factor 1 alpha promoter (EF1α) (Nakai 1998, Blood 91:4600-4607), the human synapsin 1 gene promoter (hSyn) (Kugler S. et al. Gene Ther. 2003. 10(4):337-47) or the phosphoglycerate kinase 1 promoter (PGK1) (Hannan 1993, Gene 130:233-239), the Methyl CPG Binding Protein 2 (MECP2) promoter (Adachi et al., Hum. Mol. Genetics. 2005; 14(23): 3709-3722), the human neuron-specific enolase (NSE) promoter (Twyman, R. M. and E. A. Jones (1997). J Mol Neurosci 8(1): 63-73)), the calcium/calmodulin dependent protein-kinase II (CAMKII) promoter (Nathanson, J. L., et al. (2009). Neuroscience 161(2): 441-450) and the human ubiquitin C (UBC) promoter (Schorpp, M., et al. (1996). Nucleic Acids Res 24(9): 1787-1788).

In one embodiment, said promoter comprises SEQ ID NO: 1, or preferably SEQ ID NO: 1 operably-linked in a 5′ to 3′ orientation to SEQ ID NO: 2.

In one embodiment, said promoter comprises SEQ ID NO: 3.

In one preferred embodiment, said promoter comprises SEQ ID NO: 4.

In one embodiment, said promoter comprises SEQ ID NO: 5 or SEQ ID NO: 35 or SEQ ID NO: 6, or preferably SEQ ID NO: 35 operably-linked in a 5′ to 3′ orientation to SEQ ID NO: 6.

In one embodiment, said promoter comprises SEQ ID NO: 7 or preferably SEQ ID NO: 7 operably-linked in a 5′ to 3′ orientation to SEQ ID NO: 34.

In one embodiment, said promoter comprises SEQ ID NO: 8.

In one embodiment, said promoter comprises SEQ ID NO: 9.

In one embodiment, said promoter comprises SEQ ID NO: 10.

In one embodiment, said promoter comprises SEQ ID NO: 11, or preferably SEQ ID NO: 11 operably-linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13.

In another preferred embodiment, said promoter comprises SEQ ID NO: 14.

In alternative embodiments, the nucleic acid construct comprises at least (i) a transgene encoding GAT-1 and a promoter operably-linked to said transgene, wherein the promoter is at least 90%, 91%, 92%, 93%, 94%, 95%, 96%, 97%, 98%, 99%, to:
a. SEQ ID NO: 1, or SEQ ID NO: 1 operably-linked in a 5′ to 3′ orientation to SEQ ID NO: 2; or
b. SEQ ID NO: 3 or
c. SEQ ID NO: 4; or
d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
e. SEQ ID NO: 7 or preferably SEQ ID NO: 7 operably-linked in a 5′ to 3′ orientation to SEQ ID NO: 34; or
f. SEQ ID NO: 8; or
g. SEQ ID NO: 9; or
h. SEQ ID NO: 10; or
i. SEQ ID NO: 11, or preferably SEQ ID NO: 11 operably-linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
j. SEQ ID NO: 14.

The promoter used in the nucleic acid constructs of the present invention may be a functional variant or fragment of the promoters described herein. A functional variant or fragment of the promoters described herein may be functional in the sense that it retains the characteristics of the corresponding non-variant or full-length promoter. Thus, a functional variant or fragment of the promoters described herein retains the capacity to drive the transcription of transgene to which said functional variant or fragment is operably linked, thereby driving the expression of GAT-1 encoded by said transgene. A functional variant or fragment of the promoters described herein may retain specificity for a particular tissue type. For example, a functional variant or fragment of the promoter described herein may be specific for cells of the CNS such as the endogenous hSLC6A1 promoter. A functional variant or fragment of the promoters described herein may specifically drive expression of GAT-1 in the neurons and/or the astrocytes.

The promoters used in the present invention may comprise a “minimal sequence”, which should be understood to be a nucleotide sequence of the promoter of sufficient length and which comprise the required elements to function as a promoter, i.e. capable of driving the transcription of the transgene to which said promoter is operably linked, thereby driving the expression of GAT-1.

The minimal promoter used in the nucleic acid constructs of the present invention may be a for example the promoter CAG comprising SEQ ID NO: 1 or the EF1a promoter comprising SEQ ID NO: 5 or the hDLX promoter comprising SEQ ID NO: 11.

The promoter described in the present invention may comprise one or more introns. As used herein, the term “intron” refers to a intragenic non-coding nucleotide sequence. Typically, introns are transcribed from the DNA into messenger RNA (mRNA) during transcription of a gene but are excised from the mRNA transcript by splicing prior to its translation.

The promoter used in the present invention may comprise a functional variant or fragment of an intron described herein. A functional variant or fragment of an intron described herein may be functional in the sense that it retains the characteristics of the corresponding non-variant or full-length intron. Thus, functional variants or fragments of an intron described herein are non-coding. Functional variants or fragments of an intron described herein may also retain the capacity to be transcribed from DNA to mRNA and/or the capacity to be excised from mRNA by splicing.

Introns that may be incorporated in the promoters used in the present invention may be from naturally non-coding regions or engineered.

Introns used in the present invention may be a) the chimeric intron CBA/RbG intron comprising or consisting of SEQ ID NO: 2 or a functional variant or fragment thereof having at least 90%, 91%, 92%, 93%, 94%, 95%, 96%, 97%, 98%, 99%, 99.5%, or 99.9% identity to SEQ ID NO: 2; b) the EF1a intron comprising or consisting of SEQ ID NO: 6 or a functional variant or fragment thereof having at least 90%, 91%, 92%, 93%, 94%, 95%, 96%, 97%, 98%, 99%, 99.5%, or 99.9% identity to SEQ ID NO: 6; or c) the MECP2 intron comprising or consisting of SEQ ID NO: 34 or a functional variant or fragment thereof having at least 90%, 91%, 92%, 93%, 94%, 95%, 96%, 97%, 98%, 99%, 99.5%, or 99.9% identity to SEQ ID NO: 34; or d) the hDLX intron comprising or consisting of SEQ ID NO: 13 or a functional variant or fragment thereof having at least 90%, 91%, 92%, 93%, 94%, 95%, 96%, 97%, 98%, 99%, 99.5%, or 99.9% identity to SEQ ID NO: 13.

In some embodiments, the promoters and/or introns described here may be combined with non-expressing exonic sequences. The non-expressing exonic sequences are not capable of producing a transcript rather may flank an intronic sequence to provide splice sites.

Alternatively, the promoter for use in the present invention may be a chemical inducible promoter. As used herein, a chemical inducible promoter is a promoter that is regulated by the in vivo administration of a chemical inducer to said subject in need thereof. Examples of suitable chemical inducible promoters include without limitation Tetracycline/Minocycline inducible promoter (Chtarto 2003, Neurosci Lett. 352:155-158) or rapamycin inducible systems (Sanftner 2006, Mol Ther. 13:167-174).

The nucleic acid construct according to the invention may further a 3′ untranslated region that usually contains a polyadenylation signal sequence and/or transcription terminator.

As used herein, the term “polyadenylation signal sequence” (or “polyadenylation site or “poly(A) signal” which are all used interchangeably herein) refers to a specific recognition sequence within 3′ untranslated region (3′ UTR) of the gene, which is transcribed into precursor mRNA molecule and guides the termination of the gene transcription. The polyadenylation signal sequence acts as a signal for the endonucleolytic cleavage of the newly formed precursor mRNA at its 3′-end, and for the addition to this 3′-end of a RNA stretch consisting only of adenine bases (polyadenylation process; poly(A) tail). The polyadenylation signal sequence is important for the nuclear export, translation, and stability of mRNA. In the context of the invention, the polyadenylation signal sequence is a recognition sequence that can direct polyadenylation of mammalian genes and/or viral genes, in mammalian cells.

The polyadenylation signal sequence signals typically consist of a) a consensus sequence AAUAAA, which has been shown to be required for both 3′-end cleavage and polyadenylation of pre-messenger RNA (pre-mRNA) as well as to promote downstream transcriptional termination, and b) additional elements upstream and downstream of AAUAAA that control the efficiency of utilization of AAUAAA as a poly(A) signal. There is considerable variability in these motifs in mammalian genes.

In one embodiment, optionally in combination with one or more features of the various embodiments described above or below, the polyadenylation signal sequence of the nucleic acid construct of the invention is a polyadenylation signal sequence of a mammalian gene or a viral gene. Suitable polyadenylation signals include, among others, a SV40 early polyadenylation signal, a SV40 late polyadenylation signal, a HSV thymidine kinase polyadenylation signal, a protamine gene polyadenylation signal, an adenovirus 5 EIb polyadenylation signal, a growth hormone polyadenylation signal, a PBGD polyadenylation signal, in silico designed polyadenylation signal (synthetic) and the like.

In one particular embodiment, the nucleic acid construct comprises a transgene encoding a gamma butyric acid (GABA) transporter protein 1 (GAT-1) comprising SEQ ID NO: 18, 19, 20; or a sequence having at least 95% or 96% or 97% or 98% or 99% or 99.5% sequence identity to SEQ ID NO: 18, 19 or 20 and retaining functionality as GAT-1, wherein the nucleic acid construct further comprises a promoter operably-linked to said transgene, wherein said promoter preferably comprises SEQ ID NO: 1, or preferably SEQ ID NO: 1 operably linked in a 5′ to 3′ orientation to SEQ ID NO: 2; or SEQ ID NO: 3; or SEQ ID NO: 4; or SEQ ID NO: 5, or SEQ ID NO: 35 or SEQ ID NO: 6 or preferably SEQ ID NO: 35 operably linked in a 5′ to 3′ orientation to SEQ ID NO: 6; or SEQ ID NO: 7; or SEQ ID NO: 8; or SEQ ID NO: 9; or SEQ ID NO: 10; or SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or SEQ ID NO: 14; wherein the nucleic acid construct further comprises a polyadenylation signal sequence preferably a SV40 polyadenylation signal sequence, more preferably comprising a polyadenylation signal sequence comprising SEQ ID NO: 17. Preferably, the transgene is a solute carrier family 6 member 1 (SLC6A1) gene comprising SEQ ID NO: 15, 26, 27, 28 or 29, more preferably SEQ ID NO: 15.

In a most preferred embodiment, the nucleic acid construct comprises a transgene encoding a gamma butyric acid (GABA) transporter protein 1 (GAT-1) comprising SEQ ID NO: 18, 19, 20; or a sequence having at least 95% or 96% or 97% or 98% or 99% or 99.5% sequence identity to SEQ ID NO: 18, 19 or 20 and retaining functionality as GAT-1, wherein the nucleic acid construct further comprises a promoter operably-linked to said transgene, wherein said promoter preferably comprises SEQ ID NO: 4 or SEQ ID NO: 14; wherein the nucleic acid construct further comprises a polyadenylation signal sequence, preferably a SV40 polyadenylation signal sequence, more preferably comprising a polyadenylation signal sequence comprising SEQ ID NO: 17; wherein the transgene is a solute carrier family 6 member 1 (SLC6A1) gene comprising SEQ ID NO: 15, 26, 27, 28 or 29, more preferably SEQ ID NO: 15.

In one embodiment, there is provided a nucleic acid construct comprising a transgene encoding a gamma butyric acid (GABA) transporter protein 1 (GAT-1) and retaining functionality as GAT-1, wherein the nucleic acid construct further comprises a promoter operably-linked to said transgene, wherein said promoter preferably comprises SEQ ID NO: 4 or SEQ ID NO: 14; wherein the nucleic acid construct further comprises a polyadenylation signal sequence.

In one embodiment, there is provided a nucleic acid construct comprising a transgene encoding a gamma butyric acid (GABA) transporter protein 1 (GAT-1) and retaining functionality as GAT-1, wherein the nucleic acid construct further comprises a promoter operably-linked to said transgene, wherein said promoter preferably comprises SEQ ID NO: 4 or SEQ ID NO: 14; wherein the nucleic acid construct further comprises a polyadenylation signal sequence, preferably a SV40 polyadenylation signal sequence, more preferably comprising a polyadenylation signal sequence comprising SEQ ID NO: 17.

In another embodiment, the transgene encoding a gamma butyric acid (GABA) transporter protein 1 (GAT-1) and retaining functionality as GAT-1, further comprises a promoter operably-linked to said transgene, wherein said promoter preferably comprises SEQ ID NO: 4 or SEQ ID NO: 14; wherein the nucleic acid construct further comprises a polyadenylation signal sequence, preferably a SV40 polyadenylation signal sequence, more preferably comprising a polyadenylation signal sequence comprising SEQ ID NO: 17; and wherein the transgene encoding a gamma butyric acid (GABA) transporter protein 1 (GAT-1) comprises, with reference to SEQ ID NO: 18, one or more mutations, preferably one or more mutations selected from Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; Val578Ile.

The nucleic acid construct may also comprise additional regulatory elements such as, for example, enhancer sequences, introns, microRNA targeted sequence, a polylinker sequence facilitating the insertion of a DNA fragment within a vector and/or splicing signal sequences.

The present invention further provides for a viral vector comprising the nucleic acid construct as described herein.

The term “viral vector” typically refers to the nucleic acid part of the viral particle as disclosed herein, which may be packaged in a capsid to form a viral particle for delivering into a host, such as a patient.

Viral vectors of the present invention typically comprise at least (i) a nucleic acid construct including a transgene and suitable nucleic acid elements for its expression in a host, and (ii) all or a portion of a viral genome, for example at least inverted terminal repeats of a viral genome.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

In one embodiment, the viral vector according to the present invention comprises a 5′ITR, and a 3′ITR of a virus.

In one embodiment, the viral vector comprises a 5′ITR and a 3′ITR of a virus independently selected from the group consisting of parvoviruses (in particular adeno-associated viruses), adenoviruses, alphaviruses, retroviruses (in particular gamma retroviruses, and lentiviruses), herpesviruses, and SV40; in a preferred embodiment the virus is an adeno-associated virus (AAV), an adenovirus (Ad), or a lentivirus. More preferably an AAV.

In one embodiment, the viral vector comprises a 5′ITR and a 3′ITR of an AAV.

AAV has arisen considerable interest as a potential vector for human gene therapy. Among the favourable properties of the virus are its lack of association with any human disease, its ability to infect both dividing and non-dividing cells, and the wide range of cell lines derived from different tissues that can be infected. The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length.

AAV ITRs in the viral vectors of the invention may have a wild-type nucleotide sequence or may be altered by the insertion, deletion or substitution of one or more nucleotides, typically, no more than 5, 4, 3, 2 or 1 nucleotide insertion, deletion or substitution as compared to known AAV ITRs. The serotype of the inverted terminal repeats (ITRs) of the AAV vector may be selected from any known human or non-human AAV serotype.

In specific embodiments, the viral vector may be carried out by using ITRs of any AAV serotype. Known AAV ITRs include without limitations, AAV1, AAV2, AAV3 (including types 3A and 3B), AAV-LK03, AAV4, AAV5, AAV6, AAV7, AAV8, AAV9, AAV10 (AAVrh10), AAV11, AAV12, avian AAV, bovine AAV, canine AAV, equine AAV, ovine AAV. Recombinant serotype such as Rec2 and Rec3 identified from primate brain are also included.

Alternatively, the viral vector of the invention may comprise synthetic 5′ITR and/or 3′ITR.

In one embodiment, the nucleic acid construct described above is comprised in said viral vector which further comprises a 5′ITR and a 3′ITR of an AAV of a serotype AAV2. In a particular embodiment, the viral vector comprises a 5′ITR and 3′ITR of an AAV of a serotype AAV2, preferably of SEQ ID NO: 15 and/or 16 or a sequence having at least 80% or at least 90% of identity with SEQ ID NO: 15 and/or 16.

In one embodiment, the viral vector comprising the nucleic acid construct as described herein, wherein the viral vector further comprises inverted terminal repeat (ITR) at 5′ and/or 3′ flanking said nucleic acid construct, preferably a 5′ITR and 3′ITR.

In one embodiment, the 5′ITR and/or the 3′ITR comprise the ITR of a natural adeno-associated virus (AAV), such as AAV2.

In one preferred embodiment, the 5′ITR comprises SEQ ID NO: 22 and/or the 3′ITR comprises SEQ ID NO: 23.

In one particular embodiment, the viral vector comprises a nucleic acid construct comprising a transgene encoding GAT-1 comprising:
i. SEQ ID NO: 18, 19, 20; or
ii. a sequence having at least 95% or 96% or 97% or 98% or 99% or 99.5% sequence identity to SEQ ID NO: 18, 19 or 20 and retaining functionality as GAT-1;
   wherein the nucleic acid construct further comprises a promoter operably-linked to said transgene, wherein said promoter preferably comprises:
a. SEQ ID NO: 1, or preferably SEQ ID NO: 1 operably linked in a 5′ to 3′ orientation to SEQ ID NO: 2; or
b. SEQ ID NO: 3; or
c. SEQ ID NO: 4; or
d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
e. SEQ ID NO: 7; or preferably SEQ ID NO: 7 operably-linked in a 5′ to 3′ orientation to SEQ ID NO: 34; or
f. SEQ ID NO: 8; or
g. SEQ ID NO: 9; or
h. SEQ ID NO: 10; or
i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
j. SEQ ID NO: 14;
   wherein the nucleic acid construct further comprises a polyadenylation signal sequence preferably a SV40 polyadenylation signal sequence, more preferably comprising a polyadenylation signal sequence comprising SEQ ID NO: 17; and wherein the viral vector further comprises inverted terminal repeat (ITR) at 5′ and/or 3′ flanking said nucleic acid construct, preferably a 5′ITR and 3′ITR.

In one embodiment, the 5′ITR and/or the 3′ITR comprise the ITR of a natural adeno-associated virus (AAV), such as AAV2.

In one preferred embodiment, the 5′ITR comprises SEQ ID NO: 22 and/or the 3′ITR comprises SEQ ID NO: 23.

Hence, in one preferred embodiment, the viral vector comprises a nucleic acid construct comprising a transgene encoding GAT-1 comprising:
a) SEQ ID NO: 18, 19, 20; or
b) a sequence having at least 95% or 96% or 97% or 98% or 99% or 99.5% sequence identity to SEQ ID NO: 18, 19 or 20 and retaining functionality as GAT-1; or
c) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
   wherein the nucleic acid construct further comprises a promoter operably-linked to said transgene, wherein said promoter preferably comprises:
a. SEQ ID NO: 1, or preferably SEQ ID NO: 1 operably linked in a 5′ to 3′ orientation to SEQ ID NO: 2; or
b. SEQ ID NO: 3; or
c. SEQ ID NO: 4; or
d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
e. SEQ ID NO: 7; or preferably SEQ ID NO: 7 operably-linked in a 5′ to 3′ orientation to SEQ ID NO: 34; or
f. SEQ ID NO: 8; or
g. SEQ ID NO: 9; or
h. SEQ ID NO: 10; or
i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
j. SEQ ID NO: 14;
   wherein the nucleic acid construct further comprises a polyadenylation signal sequence preferably a SV40 polyadenylation signal sequence, more preferably comprising a polyadenylation signal sequence comprising SEQ ID NO: 17; and wherein the viral vector further comprises inverted terminal repeat (ITR) at 5′ and/or 3′ flanking said nucleic acid construct, preferably a 5′ITR and 3′ITR; wherein the 5′ITR comprises SEQ ID NO: 22 and/or the 3′ITR comprises SEQ ID NO: 23. More preferably, the transgene is a solute carrier family 6 member 1 (SLC6A1) gene comprising SEQ ID NO: 15, 26, 27, 28 or 29, even more preferably SEQ ID NO: 15.

In a preferred embodiment, the invention provides for a viral vector comprising a nucleic acid construct comprising a transgene encoding:
i. a gamma butyric acid (GABA) transporter protein 1 (GAT-1) comprising SEQ ID NO: 18, 19, 20; or
ii. a sequence having at least 95% or 96% or 97% or 98% or 99% or 99.5% sequence identity to SEQ ID NO: 18, 19 or 20 and retaining functionality as GAT-1; or
iii. a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Vail 42Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
   wherein said viral vector further comprises a promoter operably-linked to said transgene, wherein said promoter preferably comprises SEQ ID NO: 4 or SEQ ID NO: 14; wherein the nucleic acid construct comprised in said viral vector comprises a polyadenylation signal sequence and wherein said viral vector further comprises inverted terminal repeat (ITR) at 5′ and/or 3′ flanking said nucleic acid construct, preferably a 5′ITR and a 3′ITR. More preferably, the transgene encodes a gamma butyric acid (GABA) transporter protein 1 (GAT-1) comprising SEQ ID NO: 18.

In a preferred embodiment, the invention provides for a viral vector comprising a nucleic acid construct comprising a transgene encoding:
i. a gamma butyric acid (GABA) transporter protein 1 (GAT-1) comprising SEQ ID NO: 18, 19, 20; or
ii. a sequence having at least 95% or 96% or 97% or 98% or 99% or 99.5% sequence identity to SEQ ID NO: 18, 19 or 20 and retaining functionality as GAT-1; or
iii. a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Vail 42Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
   wherein said viral vector further comprises a promoter operably-linked to said transgene, wherein said promoter preferably comprises SEQ ID NO: 4 or SEQ ID NO: 14; wherein the nucleic acid construct comprised in said viral vector comprises a polyadenylation signal sequence, preferably a polyadenylation signal sequence comprising SEQ ID NO: 17, and wherein said viral vector further comprises inverted terminal repeat (ITR) at 5′ and/or 3′ flanking said nucleic acid construct, preferably a 5′ITR and a 3′ITR. More preferably, the transgene encodes a gamma butyric acid (GABA) transporter protein 1 (GAT-1) comprising SEQ ID NO: 18.

In a preferred embodiment, the invention provides for a viral vector comprising a nucleic acid construct comprising a transgene which is a solute carrier family 6 member 1 (SLC6A1) gene, wherein the transgene preferably comprises:
i. SEQ ID NO: 15, 26, 27, 28 or 29, more preferably SEQ ID NO: 15;
ii. or a sequence having at least 95% or 96% or 97% or 98% or 99% or 99.5% sequence identity to SEQ ID NO: 15, 26, 27, 28 or 29.
   wherein said viral vector further comprises a promoter operably-linked to said transgene, wherein said promoter preferably comprises SEQ ID NO: 4 or SEQ ID NO: 14; wherein the nucleic acid construct comprised in said viral vector comprises a polyadenylation signal sequence and wherein said viral vector further comprises inverted terminal repeat (ITR) at 5′ and/or 3′ flanking said nucleic acid construct, preferably a 5′ITR and a 3′ITR. More preferably, the transgene encodes a gamma butyric acid (GABA) transporter protein 1 (GAT-1) comprising SEQ ID NO: 18.

In a preferred embodiment, the invention provides for a viral vector comprising a nucleic acid construct comprising a transgene which is a solute carrier family 6 member 1 (SLC6A1) gene, wherein the transgene preferably comprises:
i. SEQ ID NO: 15, 26, 27, 28 or 29, more preferably SEQ ID NO: 15;
ii. or a sequence having at least 95% or 96% or 97% or 98% or 99% or 99.5% sequence identity to SEQ ID NO: 15, 26, 27, 28 or 29.
   wherein said viral vector further comprises a promoter operably-linked to said transgene, wherein said promoter preferably comprises SEQ ID NO: 4 or SEQ ID NO: 14; wherein the nucleic acid construct comprised in said viral vector comprises a polyadenylation signal sequence, preferably a polyadenylation signal sequence comprising SEQ ID NO: 17 and wherein said viral vector further comprises inverted terminal repeat (ITR) at 5′ and/or 3′ flanking said nucleic acid construct, preferably a 5′ITR and a 3′ITR. More preferably, the transgene encodes a gamma butyric acid (GABA) transporter protein 1 (GAT-1) comprising SEQ ID NO: 18.

The transgene encoding a gamma butyric acid (GABA) transporter protein 1 (GAT-1) and retaining functionality as GAT-1, wherein the nucleic acid construct further comprises a promoter operably-linked to said transgene, wherein said promoter preferably comprises SEQ ID NO: 4 or SEQ ID NO: 14; wherein the nucleic acid construct further comprises a polyadenylation signal sequence, preferably a SV40 polyadenylation signal sequence, more preferably comprising a polyadenylation signal sequence comprising SEQ ID NO: 17; wherein the transgene encoding a gamma butyric acid (GABA) transporter protein 1 (GAT-1) comprises, with reference to SEQ ID NO: 18, one or more mutations, preferably one or more mutations selected from Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; Val578Ile.

The present invention further provides for a viral particle comprising the nucleic acid construct or the viral vector as described herein.

As used herein, the term “viral particle” relates to an infectious and typically replication-defective virus particle comprising (i) a viral vector packaged within (optionally comprising a nucleic acid construct comprising a transgene) and (ii) a capsid.

In preferred embodiments, the capsid is formed of capsid proteins of an adeno-associated virus.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. AAV serotypes comprise AAV1, AAV2, AAV3 (including A and B) AAV-LK03, AAV4, AAV5, AAV6, AAV7, AAV8, AAV9, AAV10 (AAVrh10) or AAV11, or combinations thereof, also recombinant serotypes, such as Rec2 and Rec3 identified from primate brain. In the viral particle of the invention, the capsid may be derived from any AAV serotype and combinations of serotypes (such as VP1 from an AAV and VP2 and/or VP3 from a different serotype).

In specific embodiments, examples of AAV serotypes of the capsid proteins for use in a viral particle according to the present invention comprises AAV2, AAV5, AAV8, AAV9, AAV2-retro or AAVtt.

Hence, in one embodiment, the viral particle according to the invention comprises at least a VP1 capsid protein from an AAV, wherein said capsid protein preferably comprises AAV2, AAV5, AAV6, AAV8, AAV9 (such as AAV9.hu14 comprising SEQ ID NO: 25), AAV10, AAV-true type (AAVtt such as comprising SEQ ID NO: 24) or combinations thereof.

AAVtt is described in detail in Tordo et al., Brain. 2018; 141(7): 2014-2031 and WO 2015/121501, which are incorporated herein by reference in their entirety.

Reviews of AAV serotypes and variants may be found in Choi et al (Curr Gene Ther. 2005; 5(3); 299-310) and Wu et al (Molecular Therapy. 2006; 14(3), 316-327).

In a preferred embodiment, the viral particle comprises the capsid protein from AAVtt and preferably comprises SEQ ID NO: 24 or it is at least 98.5%, preferably 99% or 99.5% identical to SEQ ID NO: 24.

In another preferred embodiment, the viral particle comprises the capsid protein from AAV9 and preferably comprises SEQ ID NO: 25 or it is at least 98.5%, preferably 99% or 99.5% identical to SEQ ID NO: 25.

AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated 5 virus 4 NC_001829; Adeno-associated virus 5 Y18065,5AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature.

The term genetic isolate describes a population of AAV viruses which has undergone limited genetic mixing with other naturally occurring AAV viruses, thereby defining a recognizably distinct population at a genetic level. Examples of clades and isolates of AAV that may be used in the invention include:
Clade A: AAV1 NC_002077, AF063497, AAV6 NC_001862, Hu. 48 AY530611, Hu 43 AY530606, Hu 44 AY530607, Hu 46 AY530609;
Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AYS30624, Hu64 AY530625, Hul3 AY530578, Hu56 AY530618, Hu57 AY530619, Hu49 AY530612, Hu58 25 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, HuT70 AY695373, Hu T40 AY695372, Hu T32 AY695371, Hu T17 AY695370, Hu LG15 AY695377;
Clade C: Hu9 AY530629, HulO AY530576, Hull AY530577, Hu53 AY530615, Hu55 AY530617, Hu54 AY530616, Hu7 AY530628, Hul8 AY530583, Hul5 AY530580, Hul6 AY530581, Hu25 AY530591, Hu60 AY530622, Ch5 AY243021, Hu3 AY530595, Hul AY530575, Hu4 AY530602 Hu2, AY530585, Hu61 AY530623;
Clade D: Rh62 AY530573, Rh48 AY530561, Rh54 AY530567, Rh55 AY530568, C5 y2 AY243020, AAV7 AF513851, Rh35 AY243000, Rh37 AY242998, Rh36 AY242999, Cy6 AY243016, Cy4 AY243018, Cy3 AY243019, Cy5 AY243017, Rhl3 AY243013;
Clade E: Rh38 AY530558, Hu66 AY530626, Hu42 AY530605, Hu67 AY530627, Hu40 AY530603, Hu41 AY530604, Hu37 AY530600, Rh40 10 AY530559, Rh2 AY243007, Bbl AY243023, Bb2 AY243022, RhlO AY243015, Hul7 AY530582, Hub AY530621, Rh25 AY530557, Pi2 AY530554, Pi1 AY530553, Pi3 AY530555, Rh57 AY530569, Rh50 AY530563, Rh49 AY530562, Hu39 AY530601, Rh58 AY530570, Rhbl AY530572, Rh52AY530565, Rh53 AY530566, Rh51 AY530564, Rh64 AY530574, Rh43 15 AY530560, AAV8 AF513852, Rh8 AY242997, Rhl AY530556; and
Clade F: Hu 14 (AAV9) AY530579, Hu31 AY530596, Hu32 AY530597; Clonal Isolate AAV5 Y18065, AF085716, AAV 3 NC_001729, AAV 3B NC_001863, AAV4 15 NC_001829, Rh34 AY243001, Rh33 AY243002, Rh32 AY243003.

The skilled person can select an appropriate serotype, variant, Glade, clone or isolate of AAV for use in the present invention on the basis of their common general knowledge. It should be understood however that the invention also encompasses use of an AAV genome of other serotypes that may not yet have been identified or characterized.

The invention encompasses the use of capsid protein sequences from different serotypes, clades, clones, or isolates of AAV within the same vector. The invention also encompasses the packaging of the genome of one serotype into the capsid of another serotype i.e. pseudotyping. Chimeric, shuffled or capsid-modified derivatives may be selected to provide one or more desired functionalities. Thus, these derivatives may display increased efficiency of gene delivery, decreased immunogenicity (humoral or cellular), an altered tropism range and/or improved targeting of a particular cell type compared to an AAV viral vector comprising a naturally occurring AAV capsid, such as that of AAV2. Increased efficiency of gene delivery may be affected by improved receptor or co-receptor binding at the cell surface, improved internalization, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid 5 sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes. Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the viral particle to a particular cell population. The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al, referenced above.

In some embodiment, a viral particle according to the invention may be prepared by encapsulating the viral vector of an AAV vector/genome derived from a particular AAV serotype or an engineered viral vector in a viral particle formed by natural Cap proteins corresponding to an AAV of the same particular serotype. Nevertheless, several techniques have been developed to modify and improve the structural and functional properties of naturally occurring viral particles (Bunning H et al. J Gene Med 2008; 10: 717-733). Thus, in another embodiment, viral particles according to the present invention includes the nucleic acid construct comprising a transgene encoding GAT-1, flanked by ITR(s) of a given AAV serotype packaged, for example, into: a) a viral particle constituted of capsid proteins derived from the same or different AAV serotype, for example AAV2 ITRs and AAV9 capsid proteins; AAV2 ITRs and AAVtt capsid proteins; b) a mosaic viral particle constituted of a mixture of capsid proteins from different AAV serotypes or mutants, for example AAV2 ITRs with a capsid formed by proteins of two or multiple AAV serotypes; c) a chimeric viral particle constituted of capsid proteins that have been truncated by domain swapping between different AAV serotypes or variants, for example AAV2 ITRs with AAV5 capsid proteins with AAV3 domains; or d) a viral particle engineered to display selective binding domains, enabling stringent interaction with target cell specific receptors.

AAV-based gene therapy targeting the CNS have already been reviewed in Pignataro D, Sucunza D, Rico A J et al., J Neural Transm 2018; 125:575-589. More specifically, the AAV particles may be selected and/or engineered to target at least neuronal and microglial cells of the brain and of the CNS.

In specific embodiments, examples of AAV serotype of the capsid proteins for use of AAV viral particle according to the present invention comprises AAV2, AAV5, AAV6, AAV8, AAV9 (such as comprising SEQ ID NO: 25), AAV10, AAV-true type (AAVtt such as comprising SEQ ID NO: 24) or combinations thereof. In more preferred embodiments, said AAV serotype of the capsid proteins are selected from AAV9 or AAVtt serotype.

AAVtt capsid also named AAV2 true-type capsid is described for example in WO2015/121501. In one embodiment, AAVtt VP1 capsid protein comprises at least one amino acid substitution with respect to the wild type AAV VP1 capsid protein at a position corresponding to one or more of the following positions in an AAV2 protein sequence (NCBI Reference sequence: YP_680426.1): 125, 151, 162, 312, 457, 492, 499, 533, 546, 548, 585, 588 and/or 593, more particularly, AAVtt comprises one or more of the following amino acid substitutions with respect to a wild type AAV2 VP1 capsid protein (NCBI Reference sequence: YP_680426.1): V125I, V151A, A162S, T205S, N312S, Q457M, S492A, E499D, F533Y, G546D, E548G, R585S, R588T and/or A593S. In one particular embodiment, AAVtt comprises four or more mutations with respect to the wild type AAV2 VP1 capsid protein at the positions 457, 492, 499 and 533.

In a particular embodiment, optionally in combination with one or more features of the various embodiments described herein, the viral particle comprises a viral vector as described above, preferably comprising a nucleic acid construct comprising a transgene encoding a gamma butyric acid (GABA) transporter protein 1 (GAT-1) comprising i) SEQ ID NO: 18, 19, 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile; and comprising capsid proteins of an AAV9 serotype or of an AAVtt serotype, preferably capsid protein of AAVtt serotype comprising SEQ ID NO: 24 or an amino acid sequence having at least 95%, 96%, 97%, 98%, preferably 98.5%, more preferably 99% or 99.5% of identity with SEQ ID NO: 24.

In another embodiment, the viral particle comprises a viral vector comprising a nucleic acid construct comprising a transgene encoding a gamma butyric acid (GABA) transporter protein 1 (GAT-1) comprising i) SEQ ID NO: 18, 19, 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Vail 42Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; Val578Ile; and comprising capsid proteins of an AAV9 serotype or of an AAVtt serotype, preferably capsid protein of AAVtt serotype comprising SEQ ID NO: 24 or an amino acid sequence having at least 95%, 96%, 97%, 98%, preferably 98.5%, more preferably 99% or 99.5% of identity with SEQ ID NO: 24.

In another particular embodiment, optionally in combination with one or more features of the various embodiments described above or below, the viral particle comprises a viral vector as described above, preferably comprising a nucleic acid construct comprising a transgene encoding a gamma butyric acid (GABA) transporter protein 1 (GAT-1) comprising i) SEQ ID NO: 18, 19, 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile, and comprising capsid proteins of an AAV9 serotype or of an AAVtt serotype, preferably capsid protein of AAV 9 serotype comprising SEQ ID NO: 25 or an amino acid sequence having at least 95%, 96%, 97%, 98%, preferably 98.5%, more preferably 99% or 99.5% of identity with SEQ ID NO: 25.

In another embodiment, the viral particle comprises a viral vector comprising a nucleic acid construct comprising a transgene encoding a gamma butyric acid (GABA) transporter protein 1 (GAT-1) comprising i) SEQ ID NO: 18, 19, 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Vail 42Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; Val578Ile; and comprising capsid proteins of an AAV9 serotype or of an AAVtt serotype, preferably capsid protein of AAV 9 serotype comprising SEQ ID NO: 25 or an amino acid sequence having at least 95%, 96%, 97%, 98%, preferably 98.5%, more preferably 99% or 99.5% of identity with SEQ ID NO: 25.

In one preferred embodiment, the viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1 or iii) a naturally-occurring variant comprising, with reference to SEQ ID NO: 18, one or more mutations as shown in Table 3;
B) a promoter operably-linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
   e. SEQ ID NO: 7; or preferably SEQ ID NO: 7 operably-linked in a 5′ to 3′ orientation to SEQ ID NO: 34
   f. SEQ ID NO: 8; or
   g. SEQ ID NO: 9; or
   h. SEQ ID NO: 10; or
   i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   j. SEQ ID NO: 14;
C) a polyadenylation signal sequence, preferably a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17;
   wherein said viral particle preferably comprises capsid proteins of AAVtt, and more preferably comprising SEQ ID NO: 24 or a sequence having at least 95%, 96%, 97%, 98%, preferably 98.5%, more preferably 99% or 99.5% identity with SEQ ID NO: 24; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

In another preferred embodiment, the viral particle comprises a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene; wherein said promoter comprises a PGK promoter or an endogenous human SLC6A1 promoter; wherein said promoter preferably comprises SEQ ID NO: 4 or SEQ ID NO: 14;
C) a polyadenylation signal sequence;
   wherein said viral particle preferably comprises capsid proteins of AAVtt, and more preferably comprising SEQ ID NO: 24 or a sequence having at least 95%, 96%, 97%, 98%, preferably 98.5%, more preferably 99% or 99.5% identity with SEQ ID NO: 24; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

In another preferred embodiment, the viral particle comprises a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene; wherein said promoter comprises a PGK promoter or an endogenous human SLC6A1 promoter; wherein said promoter preferably comprises SEQ ID NO: 4 or SEQ ID NO: 14;
C) a polyadenylation signal sequence, preferably a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17;
   wherein said viral particle preferably comprises capsid proteins of AAVtt, and more preferably comprising SEQ ID NO: 24 or a sequence having at least 95%, 96%, 97%, 98%, preferably 98.5%, more preferably 99% or 99.5% identity with SEQ ID NO: 24; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

In another preferred embodiment, the viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations as shown in Table 3;
B) a promoter operably-linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
   e. SEQ ID NO: 7; or preferably SEQ ID NO: 7 operably-linked in a 5′ to 3′ orientation to SEQ ID NO: 34
   f. SEQ ID NO: 8; or
   g. SEQ ID NO: 9; or
   h. SEQ ID NO: 10; or
   i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   j. SEQ ID NO: 14;
C) a polyadenylation signal sequence, preferably a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17;
   wherein said viral particle preferably comprises capsid proteins of AAV9, and more preferably comprising SEQ ID NO: 25 or a sequence having at least 95%, 96%, 97%, 98%, preferably 98.5%, more preferably 99% or 99.5% identity with SEQ ID NO: 25; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

In another preferred embodiment, the viral particle comprises a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene; wherein said promoter comprises a PGK promoter or an endogenous human SLC6A1 promoter; wherein said promoter preferably comprises SEQ ID NO: 4 or SEQ ID NO: 14;
C) a polyadenylation signal sequence;
   wherein said viral particle preferably comprises capsid proteins of AAV9, and more preferably comprising SEQ ID NO: 25 or a sequence having at least 95%, 96%, 97%, 98%, preferably 98.5%, more preferably 99% or 99.5% identity with SEQ ID NO: 25; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

In another preferred embodiment, the viral particle comprises a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene; wherein said promoter comprises a PGK promoter or an endogenous human SLC6A1 promoter; wherein said promoter preferably comprises SEQ ID NO: 4 or SEQ ID NO: 14;
C) a polyadenylation signal sequence, preferably a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17;
   wherein said viral particle preferably comprises capsid proteins of AAV9, and more preferably comprising SEQ ID NO: 25 or a sequence having at least 95%, 96%, 97%, 98%, preferably 98.5%, more preferably 99% or 99.5% identity with SEQ ID NO: 25; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

In another preferred embodiment, the viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence having at least 95% or 96% or 97% or 98% or 99% or 99.5% sequence identity to SEQ ID NO: 15, 26, 27, 28 or 29; or
B) a transgene encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
   wherein said viral particle comprises capsid proteins of AAVtt, preferably comprising SEQ ID NO: 24 or a sequence having at least 95%, 96%, 97%, 98%, preferably 98.5%, more preferably 99% or 99.5% identity with SEQ ID NO: 24.

In another preferred embodiment, the viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence having at least 95% or 96% or 97% or 98% or 99% or 99.5% sequence identity to SEQ ID NO: 15, 26, 27, 28 or 29; or
B) a transgene encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
   wherein said viral particle comprises capsid proteins of AAV9, preferably comprising SEQ ID NO: 25 or a sequence having at least 95%, 96%, 97%, 98%, preferably 98.5%, more preferably 99% or 99.5% identity with SEQ ID NO: 25.

The production of recombinant AAV viral particles is generally known in the art and has been described for instance in U.S. Pat. Nos. 5,173,414 and 5,139,941; WO 92/01070, WO 93/03769, Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

Production of viral particles carrying the viral vector and nucleic acid construct as described above can be performed by means of conventional methods and protocols, which are selected taking into account the structural features chosen for the actual embodiment of the viral particles to be produced.

Briefly, viral particles can be produced in a host cell, more particularly in specific virus-producing cell (packaging cell), which is transfected with the nucleic acid construct or viral vector to be packaged, in the presence of a helper vector or virus or other DNA construct(s).

The term “packaging cells” as used herein, refers to a cell or cell line which may be transfected with a nucleic acid construct or viral vector of the invention, and provides in trans all the missing functions which are required for the complete replication and packaging of a viral vector. Typically, the packaging cells express in a constitutive or inducible manner one or more of said missing viral functions. Said packaging cells can be adherent or suspension cells.

Typically, a process of producing viral particles comprises the following steps:
a) culturing a packaging cell comprising a nucleic acid construct or viral vector as described above in a culture medium; and
b) harvesting the viral particles from the cell culture supernatant and/or inside the cells.

Conventional methods can be used to produce viral particles, which consist on transient cell co-transfection with nucleic acid construct or expression vector (e.g. a plasmid) carrying the transgene encoding GAT-1; a nucleic acid construct (e.g., an AAV helper plasmid) that encodes rep and cap genes, but does not carry ITR sequences; and with a third nucleic acid construct (e.g., a plasmid) providing the adenoviral functions necessary for AAV replication. Viral genes necessary for AAV replication are referred herein as viral helper genes. Typically, said genes necessary for AAV replication are adenoviral helper genes, such as E1A, E1B, E2a, E4, or VA RNAs. Preferably, the adenoviral helper genes are of the Ad5 or Ad2 serotype.

Large-scale production of AAV particles according to the invention can also be carried out for example by infection of insect cells with a combination of recombinant baculoviruses (Urabe et al. Hum. Gene Ther. 2002; 13: 1935-1943). SF9 cells are co-infected with two or three baculovirus vectors respectively expressing AAV rep, AAV cap and the AAV vector to be packaged. The recombinant baculovirus vectors will provide the viral helper gene functions required for virus replication and/or packaging. Smith et al 2009 (Molecular Therapy, vol. 17, no. 11, pp 1888-1896) further describes a dual baculovirus expression system for large-scale production of AAV particles in insect cells.

Suitable culture media will be known to a person skilled in the art. The ingredients that compose such media may vary depending on the type of cell to be cultured. In addition to nutrient composition, osmolarity and pH are considered important parameters of culture media. The cell growth medium comprises a number of ingredients well known by the person skilled in the art, including amino acids, vitamins, organic and inorganic salts, sources of carbohydrate, lipids, trace elements (to name a few, CuSO4, FeSO4, Fe(NO3)3, ZnSO4), each ingredient being present in an amount which supports the cultivation of a cell in vitro (i.e., survival and growth of cells). Ingredients may also include different auxiliary substances, such as buffer substances (like sodium bicarbonate, Hepes, Tris or similarly performing buffers), oxidation stabilizers, stabilizers to counteract mechanical stress, protease inhibitors, animal growth factors, plant hydrolyzates, anti-clumping agents, anti-foaming agents. Characteristics and compositions of the cell growth media vary depending on the particular cellular requirements. Examples of commercially available cell growth media are: MEM (Minimum Essential Medium), BME (Basal Medium Eagle) DMEM (Dulbecco's modified Eagle's Medium), Iscoves DMEM (Iscove's modification of Dulbecco's Medium), GMEM, RPMI 1640, Leibovitz L-15, McCoy's, Medium 199, Ham (Ham's Media) F10 and derivatives, Ham F12, DMEM/F12, etc.

Further guidance for the construction and production of viral vectors for use according to the invention can be found in Viral Vectors for Gene Therapy, Methods and Protocols. Series: Methods in Molecular Biology, Vol. 737. Merten and Al-Rubeai (Eds.); 2011 Humana Press (Springer); Gene Therapy. M. Giacca. 2010 Springer-Verlag; Heilbronn R. and Weger S. Viral Vectors for Gene Transfer: Current Status of Gene Therapeutics. In: Drug Delivery, Handbook of Experimental Pharmacology 197; M. Schafer-Korting (Ed.). 2010 Springer-Verlag; pp. 143-170; Adeno-Associated Virus: Methods and Protocols. R. O. Snyder and P. Moulllier (Eds). 2011 Humana Press (Springer); Bunning H. et al. Recent developments in adeno-associated virus technology. J. Gene Med. 2008; 10:717-733; Adenovirus: Methods and Protocols. M. Chillón and A. Bosch (Eds.); Third Edition. 2014 Humana Press (Springer)

The present invention also relates to a host cell comprising a nucleic acid construct or a viral vector encoding GAT-1 as described above. More particularly, host cell according to the present invention is a specific virus-producing cell, also named packaging cell which is transfected with the a nucleic acid construct or a viral vector as described above, in the presence of a helper vector or virus or other DNA constructs and provides in trans all the missing functions which are required for the complete replication and packaging of a viral particle. Said packaging cells can be adherent or suspension cells.

For example, said packaging cells may be eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2) and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells.

As an alternative to mammalian sources, the packaging cells for producing the viral particles may be derived from avian sources such as chicken, duck, goose, quail or pheasant.

Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, the cells can be any packaging cells permissive for baculovirus infection and replication. In a particular embodiment, said cells are insect cells, such as SF9 cells (ATCC CRL-1711), Sf21 cells (IPLB-Sf21), MG1 cells (BTI-TN-MG1) or High Five™ cells (BTI-TN-5B1-4).

Accordingly, in a particular embodiment, optionally in combination with one or more features of the various embodiments described above or below, the host cell comprises:
a. a nucleic acid construct or viral vector comprising a transgene encoding human GAT-1 as described herein,
b. a nucleic acid construct, for example a plasmid, encoding AAV rep and/or cap genes which does not carry the ITR sequences; and, optionally,
c. a nucleic acid construct, for example a plasmid or virus, comprising viral helper genes.

In another aspect, the present invention relates to a host cell transduced with the viral particle described herein and the term “host cell” as used herein refers to any cell line that is susceptible to infection by a virus of interest, and amenable to culture in vitro.

In one additional aspect, the present invention therefore provides for a plasmid comprising a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
B) a promoter operably linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35 or SEQ ID NO: 6, or preferably SEQ ID NO: 35 operably linked in a 5′ to 3′ orientation to SEQ ID NO: 6; or
   e. SEQ ID NO: 7; or preferably SEQ ID NO: 7 operably linked in a 5′ to 3′ orientation to SEQ ID NO: 34; or
   f. SEQ ID NO: 8; or
   g. SEQ ID NO: 9; or
   h. SEQ ID NO: 10; or
   i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   j. SEQ ID NO: 14;
C) a polyadenylation signal sequence;
   wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In a further aspect of the invention there is provided a host cell for producing a viral particle wherein said viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
B) a promoter operably-linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
   e. SEQ ID NO: 7; or preferably SEQ ID NO: 7 operably-linked in a 5′ to 3′ orientation to SEQ ID NO: 34; or
   f. SEQ ID NO: 8; or
   g. SEQ ID NO: 9; or
   h. SEQ ID NO: 10; or
   i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   j. SEQ ID NO: 14;
C) a polyadenylation signal sequence;
   wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO:

23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In one embodiment of this aspect, the host cell further comprises:
a. a nucleic acid construct, preferably a plasmid, encoding AAV rep and/or cap genes which does not carry the ITR sequences; and, optionally
b. a nucleic acid construct, for example a plasmid or virus, comprising viral helper genes;
   wherein said AAV rep and/or cap genes encode capsid proteins of i) AAVtt, and more preferably comprising SEQ ID NO: 24 or a sequence having at least 95%, 96%, 97%, 98%, preferably 98.5%, more preferably 99% or 99.5% identity with SEQ ID NO: 24 or ii) AAV9, and more preferably comprising SEQ ID NO: 25 or a sequence having at least 95%, 96%, 97%, 98%, preferably 98.5%, more preferably 99% or 99.5% identity with SEQ ID NO: 25.

In a further aspect of the present invention, there is provided a method of producing a viral particle, the method comprising the step of:
a. culturing a host cell comprising a nucleic acid construct; and
b. harvesting the viral particles from the host cell culture media and/or inside the host cells;
   wherein a nucleic acid construct comprises:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
B) a promoter operably-linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
   e. SEQ ID NO: 7; or preferably SEQ ID NO: 7 operably-linked in a 5′ to 3′ orientation to SEQ ID NO: 34; or
   f. SEQ ID NO: 8; or
   g. SEQ ID NO: 9; or
   h. SEQ ID NO: 10; or
   i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   j. SEQ ID NO: 14;
C) a polyadenylation signal sequence;
   wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

Another aspect of the present invention relates to a pharmaceutical composition comprising a nucleic acid construct, or a viral vector, or a viral particle or a host cell described herein in combination with one or more pharmaceutical acceptable excipient, diluent or carrier.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Any suitable pharmaceutically acceptable carrier, diluent or excipient can be used in the preparation of a pharmaceutical composition (See e.g., Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Preferably, said pharmaceutical composition is formulated as a solution, more preferably as an optionally buffered saline solution. Supplementary active compounds can also be incorporated into the pharmaceutical compositions of the invention. Guidance on co-administration of additional therapeutics can for example be found in the Compendium of Pharmaceutical and Specialties (CPS) of the Canadian Pharmacists Association.

In one embodiment, the pharmaceutical composition is a composition suitable for intraparenchymal, intracerebral, intravenous, or intrathecal administration. These pharmaceutical compositions are exemplary only and do not limit the pharmaceutical compositions suitable for other parenteral and non-parenteral administration routes. The pharmaceutical compositions described herein can be packaged in single unit dosage or in multidosage forms.

In one preferred embodiment of the present invention there is provided a pharmaceutical composition comprising a viral particle in combination with one or more pharmaceutical acceptable excipient, diluent or carrier, wherein said viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
B) a promoter operably-linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   k. SEQ ID NO: 1, or preferably SEQ ID NO: 1 operably linked in a 5′ to 3′ orientation to SEQ ID NO: 2; or
   l. SEQ ID NO: 3; or
   m. SEQ ID NO: 4; or
   n. SEQ ID NO: 5 or SEQ ID NO: 35 or SEQ ID NO: 6, or preferably SEQ ID NO: 35 operably linked in a 5′ to 3′ orientation to SEQ ID NO: 6; or
   o. SEQ ID NO: 7; or preferably SEQ ID NO: 7 operably linked in a 5′ to 3′ orientation to SEQ ID NO: 34; or
   p. SEQ ID NO: 8; or
   q. SEQ ID NO: 9; or
   r. SEQ ID NO: 10; or
   s. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   t. SEQ ID NO: 14;
C) a polyadenylation signal sequence;
   wherein said viral particle preferably comprises capsid proteins of AAVtt, and more preferably comprising SEQ ID NO: 24 or a sequence having at least 95%, 96%, 97%, 98%, preferably 98.5%, more preferably 99% or 99.5% identity with SEQ ID NO: 24; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In a further preferred embodiment, there is provided a pharmaceutical composition comprising a viral particle in combination with one or more pharmaceutical acceptable excipient, diluent or carrier, wherein said viral particle comprises a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene; wherein said promoter comprises a PGK promoter or an endogenous human SLC6A1 promoter; wherein said promoter preferably comprises SEQ ID NO: 4 or SEQ ID NO: 14;
C) a polyadenylation signal sequence;
   wherein said viral particle preferably comprises capsid proteins of AAVtt, and more preferably comprising SEQ ID NO: 24 or a sequence having at least 95%, 96%, 97%, 98%, preferably 98.5%, more preferably 99% or 99.5% identity with SEQ ID NO: 24; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In another preferred embodiment, there is provided a pharmaceutical composition comprises a viral particle in combination with one or more pharmaceutical acceptable excipient, diluent or carrier, said viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
B) a promoter operably-linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35, or preferably SEQ ID NO: 35 operably-linked in a 5′ to 3′ orientation to SEQ ID NO: 6; or
   e. SEQ ID NO: 7; or preferably SEQ ID NO: 7 operably-linked in a 5′ to 3′ orientation to SEQ ID NO: 34; or
   f. SEQ ID NO: 8; or
   g. SEQ ID NO: 9; or
   h. SEQ ID NO: 10; or
   i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   j. SEQ ID NO: 14;
C) a polyadenylation signal sequence;
   wherein said viral particle preferably comprises capsid proteins of AAV9, and more preferably comprising SEQ ID NO: 25 or a sequence having at least 95%, 96%, 97%, 98%, preferably 98.5%, more preferably 99% or 99.5% identity with SEQ ID NO: 25; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In a further preferred embodiment, there is provided a pharmaceutical composition comprising a viral particle in combination with one or more pharmaceutical acceptable excipient, diluent or carrier, wherein said viral particle comprises a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene; wherein said promoter comprises a PGK promoter or an endogenous human SLC6A1 promoter; wherein said promoter preferably comprises SEQ ID NO: 4 or SEQ ID NO: 14;
C) a polyadenylation signal sequence;
   wherein said viral particle preferably comprises capsid proteins of AAV9, and more preferably comprising SEQ ID NO: 25 or a sequence having at least 95%, 96%, 97%, 98%, preferably 98.5%, more preferably 99% or 99.5% identity with SEQ ID NO: 25; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In other embodiments, the pharmaceutical composition comprises, in combination with one or more pharmaceutical acceptable excipient, diluent or carrier, a viral vector or nucleic acid construct as described herein.

An additional aspect of the present invention provides for the viral particle, viral vector or nucleic acid construct described herein for use in therapy.

In one aspect, the present invention provides for a viral particle, or a pharmaceutical composition comprising said viral particle in combination with one or more pharmaceutical acceptable excipient, diluent or carrier, said viral particle comprising a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
B) a promoter operably-linked to said transgene; wherein said promoter comprises CAG promoter, a UbC promoter, a PGK promoter, an EF1a promoter, a MECP2 promoter, a hNSE promoter, a hSyn promoter, a CamKII promoter, a hDLX promoter or a an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
   e. SEQ ID NO: 7; or preferably SEQ ID NO: 7 operably-linked in a 5′ to 3′ orientation to SEQ ID NO: 34
   f. SEQ ID NO: 8; or
   g. SEQ ID NO: 9; or
   h. SEQ ID NO: 10; or
   i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   j. SEQ ID NO: 14;
C) a polyadenylation signal sequence;
   wherein said viral particle preferably comprises capsid proteins of
1. AAVtt, and more preferably comprising SEQ ID NO: 24 or a sequence having at least 95%, 96%, 97%, 98%, preferably 98.5%, more preferably 99% or 99.5% identity with SEQ ID NO: 24; or
2. AAV9, and more preferably comprising SEQ ID NO: 25 or a sequence having at least 95%, 96%, 97%, 98%, preferably 98.5%, more preferably 99% or 99.5% identity with SEQ ID NO:
   wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23; wherein the viral particle or the pharmaceutical composition comprising the viral particle, is for use in therapy.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

Preferably, the use in therapy is for the treatment of myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof.

In one preferred embodiment, the present invention provides for a viral particle or a pharmaceutical composition comprising a viral particle in combination with one or more pharmaceutical acceptable excipient, diluent or carrier, said viral particle comprising a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
B) a promoter operably linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35, or preferably SEQ ID NO: 35 operably linked in a 5′ to 3′ orientation to SEQ ID NO: 6; or
   e. SEQ ID NO: 7; or preferably SEQ ID NO: 7 operably linked in a 5′ to 3′ orientation to SEQ ID NO: 34
   f. SEQ ID NO: 8; or
   g. SEQ ID NO: 9; or
   h. SEQ ID NO: 10; or
   i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   j. SEQ ID NO: 14;
C) a polyadenylation signal sequence;
   wherein said viral particle preferably comprises capsid proteins of
1. AAVtt, and more preferably comprising SEQ ID NO: 24 or a sequence having at least 95%, 96%, 97%, 98%, preferably 98.5%, more preferably 99% or 99.5% identity with SEQ ID NO: 24; or
2. AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23; wherein the viral particle or the pharmaceutical composition comprising the viral particle, is for use in the treatment of diseases caused by SLC6A1 impairment comprising single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In one preferred embodiment, the present invention provides for a viral particle or a pharmaceutical composition comprising said viral particle in combination with one or more pharmaceutical acceptable excipient, diluent or carrier, said viral particle comprising a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene; wherein said promoter comprises a PGK promoter, preferably comprising SEQ ID NO: 4 or an endogenous human SLC6A1 promoter, preferably comprising SEQ ID NO: 14;
C) a polyadenylation signal sequence;
   wherein said viral particle preferably comprises capsid proteins of
3. AAVtt, and more preferably comprising SEQ ID NO: 24 or a sequence having at least 95%, 96%, 97%, 98%, preferably 98.5%, more preferably 99% or 99.5% identity with SEQ ID NO: 24; or
4. AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23; wherein the viral particle or the pharmaceutical composition comprising said viral particle, is for use in the treatment of diseases caused by SLC6A1 impairment comprising single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In another preferred embodiment, the present invention provides for a viral particle or a pharmaceutical composition comprising a viral particle in combination with one or more pharmaceutical acceptable excipient, diluent or carrier, wherein said viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
B) a promoter operably linked to said transgene;
C) a polyadenylation signal sequence;
   wherein said viral particle preferably comprises capsid proteins of
1. AAVtt, and more preferably comprising SEQ ID NO: 24 or a sequence having at least 95%, 96%, 97%, 98%, preferably 98.5%, more preferably 99% or 99.5% identity with SEQ ID NO: 24; or
2. AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences; wherein the viral particle or the pharmaceutical composition comprising the viral particle, is for use in the treatment of diseases caused by SLC6A1 impairment comprising single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In another aspect, the present invention provides for a method of treating single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof, the method comprising administering to a subject a therapeutically-effective amount of a viral particle or a pharmaceutical composition comprising a viral particle in combination with one or more pharmaceutical acceptable excipient, diluent or carrier, said viral particle comprising a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434M et; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile; and
B) a promoter operably linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
   e. SEQ ID NO: 7; or preferably SEQ ID NO: 7 operably linked in a 5′ to 3′ orientation to SEQ ID NO: 34SEQ ID NO: 8; or
   f. SEQ ID NO: 9; or
   g. SEQ ID NO: 10; or
   h. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   i. SEQ ID NO: 14; and
C) a polyadenylation signal sequence;
   wherein said viral particle preferably comprises capsid proteins of AAVtt, and more preferably comprising SEQ ID NO: 24 or a sequence having at least 95%, 96%, 97%, 98%, preferably 98.5%, more preferably 99% or 99.5% identity with SEQ ID NO: 24; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In a preferred embodiment, the present invention provides fora method of treating single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof, the method comprising administering to a subject a therapeutically-effective amount of a viral particle or a pharmaceutical composition comprising a viral particle in combination with one or more pharmaceutical acceptable excipient, diluent or carrier, said viral particle comprising a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene; wherein said promoter comprises a PGK promoter, preferably comprising SEQ ID NO: 4 or an endogenous human SLC6A1 promoter, preferably comprising SEQ ID NO: 14;
C) a polyadenylation signal sequence;
   wherein said viral particle preferably comprises capsid proteins of
1. AAVtt, and more preferably comprising SEQ ID NO: 24 or a sequence having at least 95%, 96%, 97%, 98%, preferably 98.5%, more preferably 99% or 99.5% identity with SEQ ID NO: 24; or
2. AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In another preferred embodiment, the present invention provides for a method of treating single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof, the method comprising administering to a subject a therapeutically-effective amount of a viral particle or a pharmaceutical composition comprising a viral particle in combination with one or more pharmaceutical acceptable excipient, diluent or carrier, said viral particle comprising a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434M et; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260M et; Arg419His; or Val578Ile; and
B) a promoter operably linked to said transgene; and
C) a polyadenylation signal sequence;
   wherein said viral particle preferably comprises capsid proteins of
1. AAVtt, and more preferably comprising SEQ ID NO: 24 or a sequence having at least 95%, 96%, 97%, 98%, preferably 98.5%, more preferably 99% or 99.5% identity with SEQ ID NO: 24; or
2. AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

The term “subject” or “patient” as interchangeably used herein, refers to mammals. Mammalian species that can benefit from the disclosed methods of treatment or use in therapy include, but are not limited to, humans, non-human primates such as apes, chimpanzees, monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. Preferably, the term “subject” or “patient” refers to a human subject or human patient and even more preferably, said human subject or human patient is a neonate, an infant, a child or an adolescent.

A “therapeutically effective amount” refers to an amount of viral particles (comprising the transgene), optionally within a pharmaceutical formulation, or the amount of pharmaceutical formulation comprising such viral particles, which, when administered to a mammal or patient or subject, achieves the desired therapeutic result, such as one or more of the following therapeutic results:
a significant reduction in different seizure types (such as absence, atonic/“drop attacks”, myoclonus seizures, generalized seizures, simple partial seizures, febrile seizures, infantile spasms or combinations thereof);
a significant achievement of seizure freedom;
a significant reduction of developmental delay, language impairment, attention deficit hyperactivity disorder (ADHD), stereotypies, autism and ataxia features.

In a further aspect, the present invention provides for the use of a viral particle or a pharmaceutical composition comprising a viral particle in combination with one or more pharmaceutical acceptable excipient, diluent or carrier, in the manufacture of a medicament for the treatment of single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof; wherein said viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile; and
B) a promoter operably linked to said transgene; wherein said promoter comprises CAG promoter, or a UbC promoter, or a PGK promoter, or an EF1a promoter, or a MECP2 promoter, or a hNSE promoter, or a hSyn promoter, or a CamKII promoter, or a hDLX promoter or an endogenous human SLC6A1 promoter; wherein said promoter preferably comprises:
   a. SEQ ID NO: 1, or preferably SEQ ID NO: 1 operably linked in a 5′ to 3′ orientation to SEQ ID NO: 2; or
   b. SEQ ID NO: 3; or
   c. SEQ ID NO: 4; or
   d. SEQ ID NO: 5 or SEQ ID NO: 35 or SEQ ID NO: 6, or preferably SEQ ID NO: 35 operably-linked in a 5′ to 3′ orientation to SEQ ID NO: 6; or
   e. SEQ ID NO: 7; or preferably SEQ ID NO: 7 operably linked in a 5′ to 3′ orientation to SEQ ID NO: 34;
   f. SEQ ID NO: 8; or
   g. SEQ ID NO: 9; or
   h. SEQ ID NO: 10; or
   i. SEQ ID NO: 11, or SEQ ID NO: 11 operably linked in a 5′ to 3′ orientation to SEQ ID NO: 12 or preferably SEQ ID NO: 11 operably linked in a 5′ to 3′ orientation to SEQ ID NO: 12, wherein SEQ ID NO: 12 is operably linked in a 5′ to 3′ orientation to SEQ ID NO: 13; or
   j. SEQ ID NO: 14; and
C) a polyadenylation signal sequence;
   wherein said viral particle preferably comprises capsid proteins of AAVtt, and more preferably comprising SEQ ID NO: 24 or a sequence having at least 95%, 96%, 97%, 98%, preferably 98.5%, more preferably 99% or 99.5% identity with SEQ ID NO: 24; wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In a preferred embodiment, there is provided the use of a viral particle or a pharmaceutical composition comprising a viral particle in combination with one or more pharmaceutical acceptable excipient, diluent or carrier, in the manufacture of a medicament for the treatment of single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof; wherein said viral particle comprises a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene; wherein said promoter comprises a PGK promoter, preferably comprising SEQ ID NO: 4 or an endogenous human SLC6A1 promoter, preferably comprising SEQ ID NO: 14;
C) a polyadenylation signal sequence;
   wherein said viral particle preferably comprises capsid proteins of
1. AAVtt, and more preferably comprising SEQ ID NO: 24 or a sequence having at least 95%, 96%, 97%, 98%, preferably 98.5%, more preferably 99% or 99.5% identity with SEQ ID NO: 24; or
2. AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences, and wherein each of the 5′ITR and a 3′ITR sequences, independently, comprise or consist of sequences SEQ ID NO: 22 or 23 or a sequence having at least 80% or at least 90% of identity with SEQ ID NO: 22 and/or 23, wherein preferably 5′ITR comprises SEQ ID NO: 22 and/or 3′ITR comprises SEQ ID NO: 23.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In further embodiment, there is provided the use of a viral particle or a pharmaceutical composition comprising a viral particle in combination with one or more pharmaceutical acceptable excipient, diluent or carrier, in the manufacture of a medicament for the treatment of single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof; wherein said viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile; and
B) a promoter operably linked to said transgene; and
C) a polyadenylation signal sequence;
   wherein said viral particle preferably comprises capsid proteins of
1. AAVtt, and more preferably comprising SEQ ID NO: 24 or a sequence having at least 95%, 96%, 97%, 98%, preferably 98.5%, more preferably 99% or 99.5% identity with SEQ ID NO: 24; or
2. AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

The above methods and uses are particularly suitable for treating single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof.

In preferred embodiments, the methods and uses disclosed herein are preferably also for restoring GAT-1 function, more preferably, restoring GAT-1 function at the GABAergic synapses and/or along axon or neuropil or astrocytes.

In another preferred embodiment, the methods and uses disclosed herein are preferably also for decreasing seizure frequency or for restoring GAT-1 function and decreasing seizure frequency.

As used herein, disease caused by SLC6A1-impairment leading to single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof may be also identified by known genetic mutations.

In one embodiment, the disease caused by SLC6A1-impairment is associated with at least one mutation in the patient and leads to a pathological GAT-1 variant, wherein said pathological GAT-1 variants comprises a mutation or combinations of mutations.

As used herein, the term “pathological GAT-1 variant” means a variant of GAT-1 found in patient samples and identified through several methods of data collection, including clinical testing, research, and which is reported as being associated with a pathological phenotype such as any of the following: single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof

In a preferred embodiment, said mutation comprises, with reference to SEQ ID NO: 18, one or more mutation selected from the group consisting of R44W, R44Q, R50L, D52E, D52V, F53S, S56F, G63S, N66D, G75R, G79R, G79V, F92S, G94E, G105S, Q106R, G112V, Y140C, 0173Y, G232V, F270S, R277H, A288V, S295L, G297R, A305T, G307R, V323I, A334P, V342M, A357V, G362R, L366V, A367T, F385L, G393S, S456R, S459R, M487T, V511L, G550R or combinations thereof.

These mutations are also illustrated in Table 2A and Table 2B in the Example section hereinafter.

Hence, in one embodiment there is provided a viral particle or a pharmaceutical composition comprises a viral particle in combination with one or more pharmaceutical acceptable excipient, diluent or carrier, wherein the viral particle comprises a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1; or iii) a naturally-occurring variant comprising, with reference to SEQ ID NO: 18, one or more mutations, preferably selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; or Val578Ile;
B) a promoter operably-linked to said transgene;
C) a polyadenylation signal sequence;
   wherein said viral particle preferably comprises capsid proteins of i) AAVtt, and more preferably comprising SEQ ID NO: 24 or a sequence having at least 95%, 96%, 97%, 98%, preferably 98.5%, more preferably 99% or 99.5% identity with SEQ ID NO: 24; or ii) AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences; wherein the viral particle or the pharmaceutical composition comprising the viral particle, is for use in the treatment of diseases caused by SLC6A1 impairment comprising single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof; wherein said disease is caused by SLC6A1-impairment is associated with at least one mutation in the patient and leads to a pathological GAT-1 variant, wherein said pathological GAT-1 variants comprises a mutation or combinations of mutations and wherein said mutation preferably comprises, with reference to SEQ ID NO: 18, one or more mutation selected from the group consisting of R44W, R44Q, R50L, D52E, D52V, F53S, S56F, G63S, N66D, G75R, G79R, G79V, F92S, G94E, G105S, Q106R, G112V, Y140C, 0173Y, G232V, F270S, R277H, A288V, S295L, G297R, A305T, G307R, V323I, A334P, V342M, A357V, G362R, L366V, A367T, F385L, G393S, S456R, S459R, M487T, V511L, G550R or combinations thereof.

In another embodiment, the viral particle or a pharmaceutical composition comprises a viral particle in combination with one or more pharmaceutical acceptable excipient, diluent or carrier, wherein the viral particle comprises a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene, wherein said promoter is a PGK promoter, preferably comprising SEQ ID NO: 4 or an endogenous human SLC6A1 promoter, preferably comprising SEQ ID NO: 14;
C) a polyadenylation signal sequence;
   wherein said viral particle preferably comprises capsid proteins of i) AAVtt, and more preferably comprising SEQ ID NO: 24 or a sequence having at least 95%, 96%, 97%, 98%, preferably 98.5%, more preferably 99% or 99.5% identity with SEQ ID NO: 24; or ii) AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences; wherein the viral particle or the pharmaceutical composition comprising the viral particle is for use in the treatment of diseases caused by SLC6A1 impairment comprising single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof; wherein said disease is caused by SLC6A1-impairment is associated with at least one mutation in the patient and leads to a pathological GAT-1 variant, wherein said pathological GAT-1 variants comprises a mutation or combinations of mutations and wherein said mutation preferably comprises, with reference to SEQ ID NO: 18, one or more mutation selected from the group consisting of R44W, R44Q, R50L, D52E, D52V, F53S, S56F, G63S, N66D, G75R, G79R, G79V, F92S, G94E, G105S, Q106R, G112V, Y140C, 0173Y, G232V, F270S, R277H, A288V, S295L, G297R, A305T, G307R, V323I, A334P, V342M, A357V, G362R, L366V, A367T, F385L, G393S, S456R, S459R, M487T, V511L, G550R or combinations thereof.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

In another embodiment, the present invention provides for a method of treating single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof, the method comprising administering to a subject a therapeutically-effective amount of a viral particle or a pharmaceutical composition comprising a viral particle in combination with one or more pharmaceutical acceptable excipient, diluent or carrier, comprising a nucleic acid construct comprising:
A) a transgene encoding human GAT-1; wherein said transgene comprises SEQ ID NO: 15, 26, 27, 28 or 29, preferably SEQ ID NO: 15 or a sequence encoding human GAT-1, wherein human GAT-1 comprises i) SEQ ID NO: 18, 19 or 20; or ii) a sequence having at least 95% or 96% or 97% or 98% or 99% or 99.5% sequence identity to SEQ ID NO: 18, 19 or 20 and retaining functionality as GAT-1;
B) a promoter operably linked to said transgene;
C) a polyadenylation signal sequence;
   wherein said viral particle preferably comprises capsid proteins of i) AAVtt, and more preferably comprising SEQ ID NO: 24 or a sequence having at least 95%, 96%, 97%, 98%, preferably 98.5%, more preferably 99% or 99.5% identity with SEQ ID NO: 24; or ii) AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences; wherein said disease is caused by SLC6A1-impairment is associated with at least one mutation in the patient and leads to a pathological GAT-1 variant, wherein said pathological GAT-1 variants comprises a mutation or combinations of mutations and wherein said mutation preferably comprises, with reference to SEQ ID NO: 18, one or more mutation selected from the group consisting of R44W, R44Q, R50L, D52E, D52V, F53S, S56F, G63S, N66D, G75R, G79R, G79V, F92S, G94E, G105S, Q106R, G112V, Y140C, 0173Y, G232V, F270S, R277H, A288V, S295L, G297R, A305T, G307R, V323I, A334P, V342M, A357V, G362R, L366V, A367T, F385L, G393S, S456R, S459R, M487T, V511L, G550R or combination thereof.

In another embodiment, the present invention provides for a method of treating single-gene epilepsies, such as single-gene epilepsies accompanied by cognitive, motor behavioral comorbidities, early onset developmental and epileptic encephalopathy, epileptic encephalopathy, childhood onset Epilepsy Syndromes, myoclonic atonic epilepsy (MAE), MEA-like and other epilepsy indications such as Lennox Gastaut Syndrome as well as autism spectrum disorder and schizophrenia or diseases associated with impaired GABA uptake or combinations thereof, the method comprising administering to a subject a therapeutically-effective amount of a viral particle or a pharmaceutical composition comprising a viral particle in combination with one or more pharmaceutical acceptable excipient, diluent or carrier, comprising a nucleic acid construct comprising:
A) a transgene encoding human GAT-1; wherein said transgene comprises SEQ ID NO: 15, 26, 27, 28 or 29, preferably SEQ ID NO: 15;
B) a promoter operably-linked to said transgene, wherein said promoter is a PGK promoter, preferably comprising SEQ ID NO: 4 or an endogenous human SLC6A1 promoter, preferably comprising SEQ ID NO: 14;
C) a polyadenylation signal sequence;
   wherein said viral particle preferably comprises capsid proteins of i) AAVtt, and more preferably comprising SEQ ID NO: 24 or a sequence having at least 95%, 96%, 97%, 98%, preferably 98.5%, more preferably 99% or 99.5% identity with SEQ ID NO: 24; or ii) AAV9, and more preferably comprising SEQ ID NO: 25 or a sequence having at least 95%, 96%, 97%, 98%, preferably 98.5%, more preferably 99% or 99.5% identity with SEQ ID NO: 25;
   wherein said nucleic acid construct is comprised in a viral vector which further comprises a 5′ITR and a 3′ITR sequences; wherein said disease is caused by SLC6A1-impairment is associated with at least one mutation in the patient and leads to a pathological GAT-1 variant, wherein said pathological GAT-1 variants comprises a mutation or combinations of mutations and wherein said mutation preferably comprises, with reference to SEQ ID NO: 18, one or more mutation selected from the group consisting of R44W, R44Q, R50L, D52E, D52V, F53S, S56F, G63S, N66D, G75R, G79R, G79V, F92S, G94E, G105S, Q106R, G112V, Y140C, 0173Y, G232V, F270S, R277H, A288V, S295L, G297R, A305T, G307R, V323I, A334P, V342M, A357V, G362R, L366V, A367T, F385L, G393S, S456R, S459R, M487T, V511L, G550R or combination thereof.

Preferably, the polyadenylation sequence is a SV40 polyadenylation signal sequence, more preferably comprising or consisting of SEQ ID NO: 17 or a sequence having at least 95%, 96%, 97%, 98%, preferably 98.5%, more preferably 99% or 99.5% identity with SEQ ID NO: 17.

The methods of treatment and uses in the treatment described herein may be administered in combination with Valproate and any and all other potential anti-epileptic drugs (AEDs) known to date, as well as neuromodulatory-based treatments (vagus nerve stimulation, deep brain stimulation) and ketogenic diets or similar.

The dose of the therapy comprising administering the viral particle or a composition thereof further comprising one or more pharmaceutical acceptable excipient, diluent or carrier of the invention may be determined according to various parameters, especially according to the age, weight and condition of the patient to be treated; the route of administration; and the required regimen. A physician will be able to determine the required route of administration and dosage for any particular patient.

The nucleic acid constructs, viral vectors, viral particles, or pharmaceutical compositions of the invention may be administered, optionally through the use of a purpose-specific administration device, to the brain and/or the cerebrospinal fluid (CSF) of the patient. The delivery to the brain may be selected from intracerebral delivery, intraparenchymal delivery, intracortical delivery, intrahippocampal delivery, intraputaminal delivery, intracerebellar delivery, and combinations thereof. The delivery to the CSF may be selected from intra-cisterna magna delivery, intrathecal delivery, intracerebroventricular (ICV) delivery, and combinations thereof. The delivery to the brain and/or the cerebrospinal fluid (CSF) of the patient may be by injection. The injection to the brain may be selected from intracerebral injection, intraparenchymal injection, intracortical delivery, intrahippocampal delivery, intraputaminal injection, intracerebellar delivery, and combinations thereof. The delivery to the CSF may be selected from intra-cisterna magna injection, intrathecal injection, intracerebroventricular (ICV) injection, and combinations thereof.

The dose of the nucleic acid construct, vector, viral vector or pharmaceutical composition of the invention may be provided as a single dose, but may be repeated in cases where vector may not have targeted the correct region. The treatment is preferably a single injection, but repeat injections, for example in future years and/or with different AAV serotypes may be considered.

The sequences included in the present invention are shown in Table 1:

TABLE 1

| Sequence | |
|---|---|
| identifier | |
| and name | Sequence |
| SEQ ID NO: 1 | CGTTACATAACTTACGGTAAATGGCCCGCCTGGCTGACCGCCCAACGACCCCCGCCCATT |
| CAG 1.6 kb | GACGTCAATAATGACGTATGTTCCCATAGTAACGCCAATAGGGACTTTCCATTGACGTCA |
| promoter | ATGGGTGGAGTATTTACGGTAAACTGCCCACTTGGCAGTACATCAAGTGTATCATATGCC |
| | AAGTACGCCCCCTATTGACGTCAATGACGGTAAATGGCCCGCCTGGCATTATGCCCAGTA |
| | CATGACCTTATGGGACTTTCCTACTTGGCAGTACATCTACGTATTAGTCATCGCTATTACC |
| | ATGCGTCGAGGTGAGCCCCACGTTCTGCTTCACTCTCCCCATCTCCCCCCCCTCCCCACCC |
| | CCAATTTTGTATTTATTTATTTTTTAATTATTTTATGCAGCGATGGGGGCGGGGGGGGGG |
| | GGGGCGCGCGCCAGGCGGGGCGGGGCGGGGCGAGGGGCGGGGCGGGGCGAGGCGG |
| | AGAGGTGCGGCGGCAGCCAATCAGAGCGGCGCGCTCCGAAAGTTTCCTTTTATGGCGAG |
| | GCGGCGGCGGCGGCGGCCCTATAAAAAGCGAAGCGCGCGGCGGGCG |
| SEQ ID NO: 2 | GGAGTCGCTGCGTTGCCTTCGCCCCGTGCCCCGCTCCGCGCCGCCTCGCGCCGCCCGCCC |
| Chimeric | CGGCTCTGACTGACCGCGTTACTCCCACAGGTGAGCGGGCGGGACGGCCCTTCTCCCTCC |
| intron | GGGCTGTAATTAGCGCTTGGTTTAATGACGGCTCGTTTCTTTTCTGTGGCTGCGTGAAAG |
| (CBA + RbG) | CCTTAAAGGGCTCCGGGAGGGCCTTTGTGCGGGGGGGAGCGGCTCGGGGGGTGCGTG |
| | CGTGTGTGTGTGCGTGGGGAGCGCCGCGTGCGGCCCGCGCTGCCCGGCGGCTGTGAGC |
| | GCTGCGGGCGCGGCGCGGGGCTTTGTGCGCTCCGCGTGTGCGCGAGGGGAGCGCGGG |
| | CCGGGGGCGGTGCCCCGCGGTGCGGGGGGGCTGCGAGGGGAACAAAGGCTGCGTGCG |
| | GGGTGTGTGCGTGGGGGGGTGAGCAGGGGGTGTGGGCGCGGCGGTCGGGCTGTAACC |
| | CCCCCCTGGCACCCCCCTCCCCGAGTTGCTGAGCACGGCCCGGCTTCGGGTGCGGGGCT |
| | CCGTGCGGGGCGTGGCGCGGGGCTCGCCGTGCCGGGCGGGGGGTGGCGGCAGGTGG |
| | GGGTGCCGGGCGGGGCGGGGCCGCCTCGGGCCGGGGAGGGCTCGGGGGAGGGGCGC |
| | GGCGGCCCCGGAGCGCCGGCGGCTGTCGAGGCGCGGCGAGCCGCAGCCATTGCCTTTT |
| | ATGGTAATCGTGCGAGAGGGCGCAGGGACTTCCTTTGTCCCAAATCTGGCGGAGCCGAA |
| | ATCTGGGAGGCGCCGCCGCACCCCCTCTAGCGGGCGCGGGCGAAGCGGTGCGGCGCCG |
| | GCAGGAAGGAAATGGGCGGGGAGGGCCTTCGTGCGTCGCCGCGCCGCCGTCCCCTTCT |
| | CCATCTCCAGCCTCGGGGCTGCCGCAGGGGGACGGCTGCCTTCGGGGGGGACGGGGCA |
| | GGGGGGGGTTCGGCTTCTGGCGTGTGACCGGCGGCTTTAGAGCCTCTGCTAACCATGTT |
| | CATGCCTTCTTCTTTTTCCTACAG |
| SEQ ID NO: 3 | GGCCTCCGCGCCGGGTTTTGGCGCCTCCCGCGGGCGCCCCCCTCGTCACGGCGAGCGCT |
| UbC | GCCACGTCAGACGAAGGGCGCAGGAGCGTCCTGATCCTTCCGCCCGGACGCTCAGGACA |
| promoter | GCGGCCCGCTGCTCATAAGACTCGGCCTTAGAACCCCAGTATCAGCAGAAGGACATTTTA |
| | GGACGGGACTTGGGTGACTCTAGGGCACTGGTTTTCTTTCCAGAGAGCGGAACAGGCGA |
| | GGAAAAGTAGTCCCTTCTCGGCGATTCTGCGGAGGGATCTCCGTGGGGCGGTGAACGCC |
| | GATGATTATATAAGGACGCGCCGGGTGTGGCACAGCTAGTTCCGTCGCAGCCGGGATTT |
| | GGGTCGCGGTTCTTGTTTGTGGATCGCTGTGATCGTCACTTGGTGAGTAGCGGGCTGCT |
| | GGGCTGGCCGGGGCTTTCGTGGCCGCCGGGCCGCTCGGTGGGACGGAAGCGTGTGGA |
| | GAGACCGCCAAGGGCTGTAGTCTGGGTCCGCGAGCAAGGTTGCCCTGAACTGGGGGTT |
| | GGGGGGAGCGCAGCAAAATGGCGGCTGTTCCCGAGTCTTGAATGGAAGACGCTTGTGA |
| | GGCGGGCTGTGAGGTCGTTGAAACAAGGTGGGGGGCATGGTGGGCGGCAAGAACCCA |
| | AGGTCTTGAGCCCTTCGCTAATGCGGGAAAGCTCTTATTCGGGTGAGATGGGCTGGGCA |
| | CCATCTGGGGACCCTGACGTGAAGTTTGTCACTGACTGGAGAACTCGGTTTGTCGTCTGT |
| | TGCGGGGGCGGCAGTTATGGCGGTGCCGTTGGGCAGTGCACCCGTACCTTTGGGAGCG |
| | CGCGCCCTCGTCGTGTCGTGACGTCACCCGTTCTGTTGGCTTATAATGCAGGGTGGGGCC |
| | ACCTGCCGGTAGGTGTGCGGTAGGCTTTTCTCCGTCGCAGGACGCAGGGTTCGGGCCTA |
| | GGGTAGGCTCTCCTGAATCGACAGGCGCCGGACCTCTGGTGAGGGGAGGGATAAGTGA |
| | GGCGTCAGTTTCTTTGGTCGGTTTTATGTACCTATCTTCTTAAGTAGCTGAAGCTCCGGTT |
| | TTGAACTATGCGCTCGGGGTTGGCGAGTGTGTTTTGTGAAGTTTTTTAGGCACCTTTTGA |
| | AATGTAATCATTTGGGTCAATATGTAATTTTCAGTGTTAGACTTGTAAATTGTCCGCTAAA |
| | TTCTGGCCGTTTTTGGCTTTTTTGTTAGAC |
| SEQ ID NO: 4 | ACCGGTAGGCGCCAACCGGCTCCGTTCTTTGGTGGCCCCTTCGCGCCACCTTCTACTCCTC |
| PGK | CCCTAGTCAGGAAGTTCCCCCCCGCCCCGCAGCTCGCGTCGTGCAGGACGTGACAAATG |
| promoter | GAAGTAGCACGTCTCACTAGTCTCGTGCAGATGGACAGCACCGCTGAGCAATGGAAGCG |
| | GGTAGGCCTTTGGGGCAGCGGCCAATAGCAGCTTTGCTCCTTCGCTTTCTGGGCTCAGAG |
| | GCTGGGAAGGGGTGGGTCCGGGGGCGGGCTCAGGGGCGGGCTCAGGGGCGGGGCGG |
| | GCGCCCGAAGGTCCTCCGGAGGCCCGGCATTCTGCACGCTTCAAAAGCGCACGTCTGCC |
| | GCGCTGTTCTCCTCTTCCTCATCTCCGGGCCTTTCG |
| SEQ ID NO: 5 | GGCTCCGGTGCCCGTCAGTGGGCAGAGCGCACATCGCCCACAGTCCCCGAGAAGTTGG |
| EF1a | GGGGAGGGGTCGGCAATTGAACCGGTGCCTAGAGAAGGTGGCGCGGGGTAAACTGGG |
| promoter | AAAGTGATGTCGTGTACTGGCTCCGCCTTTTTCCCGAGGGTGGGGGAGAACCGTATATA |
| plus intron | AGTGCACTAGTCGCCGTGAACGTTCTTTTTCGCAACGGGTTTGCCGCCAGAACACAGGTA |
| | AGTGCCGTGTGTGGTTCCCGCGGGCCTGGCCTCTTTACGGGTTATGGCCCTTGCGTGCCT |
| | TGAATTACTTCCACCTGGCTGCAGTACGTGATTCTTGATCCCGAGCTTCGGGTTGGAAGT |
| | GGGTGGGAGAGTTCGTGGCCTTGCGCTTAAGGAGCCCCTTCGCCTCGTGCTTGAGTTGT |
| | GGCCTGGCCTGGGCGCTGGGGCCGCCGCGTGCGAATCTGGTGGCACCTTCGCGCCTGTC |
| | TCGCTGCTTTCGATAAGTCTCTAGCCATTTAAAATTTTTGATGACCTGCTGCGACGCTTTTT |
| | TTCTGGCAAGATAGTCTTGTAAATGCGGGCCAAGATCAGCACACTGGTATTTCGGTTTTT |
| | GGGGCCGCGGGCGGCGACGGGGCCCGTGCGTCCCAGCGCACATGTTCGGCGAGGCGG |
| | GGCCTGCGAGCGCGGCCACCGAGAATCGGACGGGGGTAGTCTCAAGCTGCCCGGCCTG |
| | CTCTGGTGCCTGGCCTCGCGCCGCCGTGTATCGCCCCGCCCTGGGCGGCAAGGCTGGCC |
| | CGGTCGGCACCAGTTGCGTGAGCGGAAAGATGGCCGCTTCCCGGCCCTGCTGCAGGGA |
| | GCACAAAATGGAGGACGCGGCGCTCGGGAGAGCGGGCGGGTGAGTCACCCACACAAA |
| | GGAAAAGGGCCTTTCCGTCCTCAGCCGTCGCTTCATGTGACTCCACGGAGTACCGGGCG |
| | CCGTCCAGGCACCTCGATTAGTTCTCCAGCTTTTGGAGTACGTCGTCTTTAGGTTGGGGG |
| | GAGGGGTTTTATGCGATGGAGTTTCCCCACACTGAGTGGGTGGAGACTGAAGTTAGGCC |
| | AGCTTGGCACTTGATGTAATTCTCCTTGGAATTTGCCCTTTTTGAGTTTGGATCTTGGTTC |
| | ATTCTCAAGCCTCAGACAGTGGTTCAAAGTTTTTTTCTTCCATTTCAGGTGTCGTGA |
| SEQ ID NO: 6 | GTAAGTGCCGTGTGTGGTTCCCGCGGGCCTGGCCTCTTTACGGGTTATGGCCCTTGCGTG |
| EF1a intron | CCTTGAATTACTTCCACCTGGCTGCAGTACGTGATTCTTGATCCCGAGCTTCGGGTTGGA |
| | AGTGGGTGGGAGAGTTCGTGGCCTTGCGCTTAAGGAGCCCCTTCGCCTCGTGCTTGAGT |
| | TGTGGCCTGGCCTGGGCGCTGGGGCCGCCGCGTGCGAATCTGGTGGCACCTTCGCGCCT |
| | GTCTCGCTGCTTTCGATAAGTCTCTAGCCATTTAAAATTTTTGATGACCTGCTGCGACGCT |
| | TTTTTTCTGGCAAGATAGTCTTGTAAATGCGGGCCAAGATCAGCACACTGGTATTTCGGT |
| | TTTTGGGGCCGCGGGCGGCGACGGGGCCCGTGCGTCCCAGCGCACATGTTCGGCGAGG |
| | CGGGGCCTGCGAGCGCGGCCACCGAGAATCGGACGGGGGTAGTCTCAAGCTGCCCGGC |
| | CTGCTCTGGTGCCTGGCCTCGCGCCGCCGTGTATCGCCCCGCCCTGGGCGGCAAGGCTG |
| | GCCCGGTCGGCACCAGTTGCGTGAGCGGAAAGATGGCCGCTTCCCGGCCCTGCTGCAGG |
| | GAGCACAAAATGGAGGACGCGGCGCTCGGGAGAGCGGGCGGGTGAGTCACCCACACA |
| | AAGGAAAAGGGCCTTTCCGTCCTCAGCCGTCGCTTCATGTGACTCCACGGAGTACCGGG |
| | CGCCGTCCAGGCACCTCGATTAGTTCTCCAGCTTTTGGAGTACGTCGTCTTTAGGTTGGG |
| | GGGAGGGGTTTTATGCGATGGAGTTTCCCCACACTGAGTGGGTGGAGACTGAAGTTAG |
| | GCCAGCTTGGCACTTGATGTAATTCTCCTTGGAATTTGCCCTTTTTGAGTTTGGATCTTGG |
| | TTCATTCTCAAGCCTCAGACAGTGGTTCAAAGTTTTTTTCTTCCATTTCAG |
| SEQ ID NO: 7 | AGCTGAATGGGGTCCGCCTCTTTTCCCTGCCTAAACAGACAGGAACTCCTGCCAATTGAG |
| MECP2 | GGCGTCACCGCTAAGGCTCCGCCCCAGCCTGGGCTCCACAACCAATGAAGGGTAATCTC |
| promoter | GACAAAGAGCAAGGGGTGGGGCGCGGGCGCGCAGGTGCAGCAGCACACAGGCTGGTC |
| | GGGAGGGCGGGGCGCGACGTCTGCCGTGCGGGGTCCCGGCATCGGTTGCGCGC |
| SEQ ID NO: 8 | ATGCAGCTGGACCTAGGAGAGAAGCAGGAGAGGAAGATCCAGCACAAAAAATCCGAAG |
| hNSE | CTAAAAACAGGACACAGAGATGGGGGAAGAAAAGAGGGCAGAGTGAGGCAAAAAGAG |
| promoter | ACTGAAGAGATGAGGGTGGCCGCCAGGCACTTTAGATAGGGGAGAGGCTTTATTTACCT |
| | CTGTTTGTTTTTTTTTTTTTTTTTTTTTTTTTTTTTTTGCGAGGTAGTCTTGCTTAGTCTCCAG |
| | GCTGGAGTGCAGTGGCACAATCTCAGCTCACTGCAACTTCCACCTCCTGGGTTCAAGCAA |
| | TTCTCCTGCCTCAGCCTCCCGAGTAGCTGGGACTACAGGCGCATGCAACCGCGCCTGGCT |
| | AATTTTTGTATTTTTAGTAGAAACGGGGTTTCACCACGTTAGCCAGGATGGTCTGGATCT |
| | CCTGACCTCGTGATCTGCCCGCCTCCGCCTTCCAAAGTGCTGGGATTACAGGGGTGAGCC |
| | ACAGCGCCTGGTCCCTATTTACTTCTGTCTTCTACCTCCAGGAGATCAAAGACGCTGGCCT |
| | TCAGACCTGATCAGACTCCCAGGGGCAGCCACCACATGTATGACAGAGAACAGAGGATG |
| | CCTGTTTTTGCCCCAAAGCTGGAAATTCATCACAACCTGAGGCCCAGGATCTGCTCTGTG |
| | CCGGTCCTCTGGGCAGTGTGGGGTGCAGAATGGGGTGCCTAGGCCTGAGCGTTGCCTG |
| | GAGCCTAGGCCGGGGGCCGCCCTCGGGCAGGCGTGGGTGAGAGCCAAGACCGCGTGG |
| | GCCGCGGGGTGCTGGTAGGAGTGGTTGGAGAGACTTGCGAAGGCGGCTGGGGTGTTC |
| | GGATTTCCAATAAAGAAACAGAGTGATGCTCCTGTGTCTGACCGGGTTTGTGAGACATT |
| | GAGGCTGTCTTGGGCTTCACTGGCAGTGTGGGCCTTCGTACCCGGGCTACAGGGGTGCG |
| | GCTCTGCCTGTTACTGTCGAGTGGGTCGGGCCGTGGGTATGAGCGCTTGTGTGCGCTGG |
| | GGCCAGGTCGTGGGTGCCCCCACCCTTCCCCCATCCTCCTCCCTTCCCCACTCCACCCTCG |
| | TCGGTCCCCCACCCGCGCTCGTACGTGCGCCTCCGCCGGCAGCTCCTGACTCATCGGGGG |
| | CTCCGGGTCACATGCGCCCGCGCGGCCCTATAGGCGCCTCCTCCGCCCGCCGCCCGGGA |
| | GCCGCAGCCGCCGCCGCCACTGCCACTCCCGCTCTCTCAGCGCCGCCGTCGCCACCGCCA |
| | CCGCCACCGCCACTACCACCGAGATCTGCGATCTAAGTAAGCTTGGCATTCCGGTACTGT |
| | TGGTAAAGCC |
| SEQ ID NO: 9 | AGTGCAAGTGGGTTTTAGGACCAGGATGAGGCGGGGTGGGGGTGCCTACCTGACGACC |
| hSyn | GACCCCGACCCACTGGACAAGCACCCAACCCCCATTCCCCAAATTGCGCATCCCCTATCA |
| promoter | GAGAGGGGGAGGGGAAACAGGATGCGGCGAGGCGCGTGCGCACTGCCAGCTTCAGCA |
| | CCGCGGACAGTGCCTTCGCCCCCGCCTGGCGGCGCGCGCCACCGCCGCCTCAGCACTGA |
| | AGGCGCGCTGACGTCACTCGCCGGTCCCCCGCAAACTCCCCTTCCCGGCCACCTTGGTCG |
| | CGTCCGCGCCGCCGCCGGCCCAGCCGGACCGCACCACGCGAGGCGCGAGATAGGGGGG |
| | CACGGGCGCGACCATCTGCGCTGCGGCGCCGGCGACTCAGCGCTGCCTCAGTCTGCGGT |
| | GGGCAGCGGAGGAGTCGTGTCGTGCCTGAGAGCGCAG |
| SEQ ID NO: | CATTATGGCCTTAGGTCACTTCATCTCCATGGGGTTCTTCTTCTGATTTTCTAGAAAATGA |
| 10 | GATGGGGGTGCAGAGAGCTTCCTCAGTGACCTGCCCAGGGTCACATCAGAAATGTCAGA |
| CamKII | GCTAGAACTTGAACTCAGATTACTAATCTTAAATTCCATGCCTTGGGGGCATGCAAGTAC |
| promoter | GATATACAGAAGGAGTGAACTCATTAGGGCAGATGACCAATGAGTTTAGGAAAGAAGA |
| | GTCCAGGGCAGGGTACATCTACACCACCCGCCCAGCCCTGGGTGAGTCCAGCCACGTTC |
| | ACCTCATTATAGTTGCCTCTCTCCAGTCCTACCTTGACGGGAAGCACAAGCAGAAACTGG |
| | GACAGGAGCCCCAGGAGACCAAATCTTCATGGTCCCTCTGGGAGGATGGGTGGGGAGA |
| | GCTGTGGCAGAGGCCTCAGGAGGGGCCCTGCTGCTCAGTGGTGACAGATAGGGGTGAG |
| | AAAGCAGACAGAGTCATTCCGTCAGCATTCTGGGTCTGTTTGGTACTTCTTCTCACGCTAA |
| | GGTGGCGGTGTGATATGCACAATGGCTAAAAAGCAGGGAGAGCTGGAAAGAAACAAG |
| | GACAGAGACAGAGGCCAAGTCAACCAGACCAATTCCCAGAGGAAGCAAAGAAACCATT |
| | ACAGAGACTACAAGGGGGAAGGGAAGGAGAGATGAATTAGCTTCCCCTGTAAACCTTA |
| | GAACCCAGCTGTTGCCAGGGCAACGGGGCAATACCTGTCTCTTCAGAGGAGATGAAGTT |
| | GCCAGGGTAACTACATCCTGTCTTTCTCAAGGACCATCCCAGAATGTGGCACCCACTAGC |
| | CGTTACCATAGCAACTGCCTCTTTGCCCCACTTAATCCCATCCCGTCTGTTAAAAGGGCCC |
| | TATAGTTGGAGGTGGGGGAGGTAGGAAGAGCGATGATCACTTGTGGACTAAGTTTGTTC |
| | GCATCCCCTTCTCCAACCCCCTCAGTACATCACCCTGGGGGAACAGGGTCCACTTGCTCCT |
| | GGGCCCACACAGTCCTGCAGTATTGTGTATATAAGGCCAGGGCAAAGAGGAGCAGGTTT |
| | TAAAGTGAAAGGCAGGCAGGTGTTGGGGAGGCAGTTACCGGGGCAACGGGAACAGGG |
| | CGTTTCGGAGGTGGTTGCCATGGGGACCTGGATGCTGACGAAGGCTCGCGAGGCTGTG |
| | AGCAGCCACAGTGCCCTGCTCAGAAGCCCCAAGCTCGTCAGTCAAGCCGGTTCTCCGTTT |
| | GCACTCAGGAGCACGGGCAGGCGAGTGGCCCCTAGTTCTGGGGGCAGC |
| SEQ ID NO: | TTCAGAATGTTATGCACTCACAGTGGTTTGGCATGCATCTGGTGAATTTTTTTTAACGAAA |
| 11 | AATTAGTGTTGGTTTCGATGTATGGTAGCATTCTCCCTAACGTAATTTGAATAATTCAGCA |
| hDLX | AAGCCCCACTACCAGCTGTACTTCTGCAGCCTCTTCCATTCTTTTCAGCATTATAATTTTGG |
| promoter | TTAATTTTCAATTTTAGGTCCTACGTCTCTGCAATTTGTGTATGAATAACAGAATAATTTCC |
| | CTCTTTTGTTTCGCCTTTCCTGTTCCTGAATCTAAATAAAGATGGCTTTTTAGTATTAAAAG |
| | TGGAAGAAAATTACAGGTAATTATCTTTGACGGTAAAAACGCTGTAATCAGCGGGCTAC |
| | ATGAAAAATTACTCTAATTATGGCTGCATTTAAGAGAATGGAAAAAAACCTTCTTGTGGA |
| | TAAAAACCTTAAATTGTCCCCAATGTCTGCTTCAAATTGGATGGCACTGCAGCTGGAGGC |
| | TTTGTTCAGAATTGATCCTGGGGAGCTACGAACCCAAAGTTTCACAGTAGGAAG |
| SEQ ID NO: | CTGGGCATAAAAGTCAGGGCAGAGCCATCTATTGCTTACATTTGCTTCT |
| 12 | |
| pBGlobin | |
| SEQ ID NO: | GTAAGTATCAAGGTTACAAGACAGGTTTAAGGAGACCAATAGAAACTGGGCTTGTCGAG |
| 13 hDLX | ACAGAGAAGACTCTTGCGTTTCTGATAGGCACCTATTGGTCTTACTGACATCCACTTTGCC |
| intron | TTTCTCTCCACAG |
| SEQ ID NO: | AGAATCCCTCAAACCTCAAGAACTGAGAGAAGGGTGTCTGGGGCTCCTGCCACCATCCC |
| 14 | TGTTTCCCTTTTAAGTAATCTGTTTCCCCATCTGTCCATCCATACACACAGCCACTTGTGTC |
| Endogenous | TCCATGACCAACCGCTGGCAGTGGAAGGGTGTCCTTCCCACCCCCACTCTTACACACACT |
| hSLC6A1 | CCCAGCTGGTACCCAGAGCCTGGTCACCCCAGGCCAGGCCTGTGTTTCCAGGTGTAACG |
| promoter | GGCAGCAGACGCTGCCCTAGGACTAGAGCAGGGAGGGGGCACGGGCCCACCCCAACCC |
| | ACAGCGACCCACAGAGGGCGAAAAGAGGACGACCGCAGAGAGAAACGGAAAGGACAG |
| | GCCAACGGAAGCAGTACTGCAAGGCTGGAAGGAGAAAAGCCAGGAGGGGAGTGCTTG |
| | CTGTGAAAGACAGGGAGACAGAGACCAAGACGGACAGGCAGACAGGCTGGTGACCCA |
| | GGATGAGGCCGGAAAGAGCCATCAAAGGAAGGAGAAGGAAGGAGAGAGATTGGAGC |
| | GGGACGGCGGGGCAGGCGAGGGAAGGAGGGGGTGGGGAGAGGGAGGGAGGAAGAG |
| | AGGGGAGAAAGAGGGAGGAAGAGAGGGGAGAAGGAGGGAGAAGAGAGCGGGAGAA |
| | TGCGAGAGGAAAGAAGGGAGAGGGGAGGCGTAGAAGGGGAGAGGAGGTGAAGGGA |
| | AAAGGAGAGAGCCTGCTGGCGGCGAAGCTGCAAGAGGCAGCTGCGGAGGGAGCGCGC |
| | GGCGGGCCTGGGGGAGCGCTGGGCGGGGGCGGGCGGTGCGGGCAGGGCTATACCCG |
| | AGCTGGGCGGGCTCCGGGCGCCGCGGGCCCTGCCCTCCCCCTCCATCCCTCCGGACTCG |
| | CTCCCCCCTCCTCTCCCTTCCCCGCGACCCTCCGCCCGCCCTCGGAAGACCGAGACAGCG |
| | GAGAGGTTGCGGGTGAGCTGCGCTGAGCCCAGGAGCCGAGGAGTCGGGAGCGCAGTA |
| | GCGCTGAGCCCGAGCCCGAGCGGCCCCGCGTCCCGAGCGCATCGGAGCGGCCGAGCCG |
| | CCCGGATGCAGCGCCTGTCCCGGGCAGCGCAGCCCCGGCCGCAGGATCTCACCCAGGGT |
| | GGCAGAAGGAGGCCTTCTGGAGCTGACCCACCCCCGACGACCATCAGGGTGAGGCAAC |
| | TCCAAGGTCCTACTCTCTTTCTGTGCCTGTTACCCACCCCGTCCTCCTAGGGTGCCCTTGA |
| | GCCGCAAAACTGCTGTCCACGTGGACCGGGGGTGACATCGCACGTCCATCTGCCAGGAC |
| | CCCTGCGTCCAAATTCCGAGAC |
| SEQ ID NO: | ATGGCGACCAACGGCAGCAAGGTGGCCGACGGGCAGATCTCCACCGAGGTCAGCGAGG |
| 15 | CCCCTGTGGCCAATGACAAGCCCAAAACCTTGGTGGTCAAGGTGCAGAAGAAGGCGGC |
| Human | AGACCTCCCCGACCGGGACACGTGGAAGGGCCGCTTCGACTTCCTCATGTCCTGTGTGG |
| SLC6A1 NCBI | GCTATGCCATCGGCCTGGGCAACGTCTGGAGGTTCCCCTATCTCTGCGGGAAAAATGGT |
| NM_003042.4 | GGGGGAGCCTTCCTGATCCCCTATTTCCTGACACTCATCTTTGCGGGGGTCCCACTCTTCC |
| (transcript | TGCTGGAGTGCTCCCTGGGCCAGTACACCTCCATCGGGGGGCTAGGGGTATGGAAGCTG |
| variant 1) | GCTCCTATGTTCAAGGGCGTGGGCCTTGCGGCTGCTGTGCTATCATTCTGGCTGAACATC |
| | TACTACATCGTCATCATCTCCTGGGCCATTTACTACCTGTACAACTCCTTCACCACGACACT |
| | GCCGTGGAAACAGTGCGACAACCCCTGGAACACAGACCGCTGCTTCTCCAACTACAGCA |
| | TGGTCAACACTACCAACATGACCAGCGCTGTGGTGGAGTTCTGGGAGCGCAACATGCAT |
| | CAGATGACGGACGGGCTGGATAAGCCAGGTCAGATCCGCTGGCCACTGGCCATCACGCT |
| | GGCCATCGCCTGGATCCTTGTGTATTTCTGTATCTGGAAGGGTGTTGGCTGGACTGGAAA |
| | GGTGGTCTACTTTTCAGCCACATACCCCTACATCATGCTGATCATCCTGTTCTTCCGTGGA |
| | GTGACGCTGCCCGGGGCCAAGGAGGGCATCCTCTTCTACATCACACCCAACTTCCGCAA |
| | GCTGTCTGACTCCGAGGTGTGGCTGGATGCGGCAACCCAGATCTTCTTCTCATACGGGCT |
| | GGGCCTGGGGTCCCTGATCGCTCTCGGGAGCTACAACTCTTTCCACAACAATGTCTACAG |
| | GGACTCCATCATCGTCTGCTGCATCAATTCGTGCACCAGCATGTTCGCAGGATTCGTCATC |
| | TTCTCCATCGTGGGCTTCATGGCCCATGTCACCAAGAGGTCCATTGCTGATGTGGCGGCC |
| | TCAGGCCCCGGGCTGGCGTTCCTGGCATACCCAGAGGCGGTGACCCAGCTGCCTATCTC |
| | CCCACTCTGGGCCATCCTCTTCTTCTCCATGCTGTTGATGCTGGGCATTGACAGCCAGTTC |
| | TGCACTGTGGAGGGCTTCATCACAGCCCTGGTGGATGAGTACCCCAGGCTCCTCCGCAA |
| | CCGCAGAGAGCTCTTCATTGCTGCTGTCTGCATCATCTCCTACCTGATCGGTCTCTCTAAC |
| | ATCACTCAGGGGGGTATTTATGTCTTCAAACTCTTTGACTACTACTCTGCCAGTGGCATGA |
| | GCCTGCTGTTCCTCGTGTTCTTTGAATGTGTCTCTATTTCCTGGTTTTACGGTGTCAACCGA |
| | TTCTATGACAATATCCAAGAGATGGTTGGATCCAGGCCCTGCATCTGGTGGAAACTCTGC |
| | TGGTCTTTCTTCACACCAATCATTGTGGCGGGCGTGTTCATTTTCAGTGCTGTGCAGATGA |
| | CGCCACTCACCATGGGAAACTATGTTTTCCCCAAGTGGGGCCAGGGTGTGGGCTGGCTG |
| | ATGGCTCTGTCTTCCATGGTCCTCATCCCCGGGTACATGGCCTACATGTTCCTCACCTTAA |
| | AGGGCTCCCTGAAGCAGCGCATCCAAGTCATGGTCCAGCCCAGCGAAGACATCGTTCGC |
| | CCAGAGAATGGTCCTGAGCAGCCCCAGGCGGGCAGCTCCACCAGCAAGGAGGCCTACA |
| | TCTAG |
| SEQ ID NO: | ATGGCGACTGACAACAGCAAGGTGGCTGATGGGCAGATCTCTACTGAGGTCAGCGAGG |
| 16 | CCCCTGTGGCCAGCGACAAGCCCAAAACCCTGGTAGTCAAGGTGCAGAAGAAGGCCGG |
| Mouse | GGACCTCCCTGACCGGGACACATGGAAGGGACGCTTCGACTTCCTCATGTCCTGCGTGG |
| SLC6A1 | GCTATGCCATCGGCCTGGGCAATGTGTGGAGGTTCCCTTACCTCTGTGGGAAAAACGGT |
| | GGCGGGGCCTTCCTAATCCCATATTTCCTGACGCTCATCTTTGCGGGTGTTCCTCTCTTCC |
| | TTTTGGAGTGCTCCCTAGGCCAGTACACCTCCATTGGGGGCCTGGGCGTATGGAAGCTG |
| | GCGCCCATGTTCAAGGGTGTGGGCCTCGCGGCAGCTGTGCTGTCCTTCTGGCTGAACATC |
| | TACTACATCGTCATCATCTCCTGGGCCATCTACTACCTGTACAACTCCTTCACCACGACCCT |
| | GCCATGGAAACAGTGTGACAACCCGTGGAACACTGACCGCTGCTTCTCCAACTACAGCCT |
| | GGTCAATACCACCAACATGACCAGCGCCGTGGTGGAGTTCTGGGAGCGCAACATGCACC |
| | AGATGACAGATGGACTGGACAAGCCAGGACAGATCCGCTGGCCTCTGGCCATCACACTG |
| | GCCATTGCCTGGGTGCTCGTGTATTTCTGCATCTGGAAGGGTGTTGGTTGGACTGGAAA |
| | GGTGGTCTACTTCTCAGCCACGTACCCCTACATCATGCTTATCATCCTGTTCTTCCGTGGA |
| | GTGACGCTTCCCGGGGCCAAGGAGGGGATCCTCTTCTACATCACACCCAACTTCCGAAA |
| | GCTGTCTGATTCTGAGGTGTGGCTTGACGCCGCCACCCAGATCTTCTTCTCCTACGGGCT |
| | GGGCCTGGGGTCCCTGATTGCTCTGGGAAGCTACAACTCTTTCCACAACAATGTGTACAG |
| | GGACTCCATCATCGTTTGCTGCATCAACTCCTGCACCAGCATGTTTGCCGGATTCGTCATC |
| | TTCTCCATCGTGGGCTTCATGGCTCATGTCACCAAGAGGTCCATAGCTGATGTGGCAGCC |
| | TCAGGCCCGGGGCTGGCATTCTTGGCGTACCCTGAGGCTGTGACACAGCTACCCATCTCT |
| | CCCCTCTGGGCTATCCTCTTCTTCTCCATGCTGCTGATGCTGGGCATTGACAGCCAGTTCT |
| | GTACCGTGGAGGGCTTCATCACTGCCCTGGTGGACGAGTACCCCAGACTTCTCCGCAATC |
| | GCCGTGAACTCTTCATTGCTGCCGTGTGCATCGTGTCCTACCTGATTGGCCTGTCTAACAT |
| | CACCCAGGGTGGCATTTATGTCTTCAAACTGTTTGATTATTACTCTGCCAGCGGCATGAG |
| | CTTGCTGTTCCTGGTTTTCTTCGAGTGTGTCTCCATTTCCTGGTTTTATGGTGTCAACCGGT |
| | TCTATGACAACATCCAGGAGATGGTTGGCTCCAGGCCCTGCATCTGGTGGAAGCTGTGC |
| | TGGTCCTTTTTCACACCCATCATTGTGGCGGGCGTGTTTCTCTTCAGTGCTGTGCAGATGA |
| | CACCACTCACCATGGGAAGCTATGTTTTCCCCAAGTGGGGCCAGGGCGTGGGCTGGCTC |
| | ATGGCTCTGTCCTCCATGGTGCTCATCCCCGGGTACATGGCTTACATGTTCCTCACCCTGA |
| | AGGGCTCCCTGAAGCAGCGTCTCCAGGTCATGATTCAGCCCAGTGAAGATATTGTGCGC |
| | CCTGAGAATGGCCCTGAGCAGCCGCAGGCTGGCAGCTCAGCCAGCAAGGAGGCCTACA |
| | TCTAG |
| SEQ ID NO: | GATCCAGACATGATAAGATACATTGATGAGTTTGGACAAACCACAACTAGAATGCAGTG |
| 17 | AAAAAAATGCTTTATTTGTGAAATTTGTGATGCTATTGCTTTATTTGTAACCATTATAAGC |
| SV40 poly A | TGCAATAAACAAGTT |
| SEQ ID NO: | MATNGSKVADGQISTEVSEAPVANDKPKTLVVKVQKKAADLPDRDTWKGRFDFLMSCVGY |
| 18 | AIGLGNVWRFPYLCGKNGGGAFLIPYFLTLIFAGVPLFLLECSLGQYTSIGGLGVWKLAPMFK |
| Human GAT-1 | GVGLAAAVLSFWLNIYYIVIISWAIYYLYNSFTTTLPWKQCDNPWNTDRCFSNYSMVNTTN |
| isoform a | MTSAVVEFWERNMHQMTDGLDKPGQIRWPLAITLAIAWILVYFCIWKGVGWTGKVVYFSATY |
| | PYIMLIILFFRGVTLPGAKEGILFYITPNFRKLSDSEVWLDAATQIFFSYGLGLGSLIALGSYN |
| | SFHNNVYRDSIIVCCINSCTSMFAGFVIFSIVGFMAHVTKRSIADVAASGPGLAFLAYPEAVTQ |
| | LPISPLWAILFFSMLLMLGIDSQFCTVEGFITALVDEYPRLLRNRRELFIAAVCIISYLIGLSN |
| | ITQGGIYVFKLFDYYSASGMSLLFLVFFECVSISWFYGVNRFYDNIQEMVGSRPCIWWKLCWSF |
| | FTPIIVAGVFIFSAVQMTPLTMGNYVFPKWGQGVGWLMALSSMVLIPGYMAYMFLTLK |
| | GSLKQRIQVMVQPSEDIVRPENGPEQPQAGSSTSKEAYI |
| SEQ ID NO: | MVNTTNMTSAVVEFWERNMHQMTDGLDKPGQIRWPLAITLAIAWILVYFCIWKGVGWTGKV |
| 19 | VYFSATYPYIMLIILFFRGVTLPGAKEGILFYITPNFRKLSDSEVWLDAATQIFFSYGLGLGS |
| Human GAT-1 | LIALGSYNSFHNNVYRDSIIVCCINSCTSMFAGFVIFSIVGFMAHVTKRSIADVAASGPGLAFLA |
| isoform b | YPEAVTQLPISPLWAILFFSMLLMLGIDSQFCTVEGFITALVDEYPRLLRNRRELFIAAVCIISY |
| | LIGLSNITQGGIYVFKLFDYYSASGMSLLFLVFFECVSISWFYGVNRFYDNIQEMVGSRPCIWWK |
| | LCWSFFTPIIVAGVFIFSAVQMTPLTMGNYVFPKWGQGVGWLMALSSMVLIPGYMAYMFL |
| | TLKGSLKQRIQVMVQPSEDIVRPENGPEQPQAGSSTSKEAYI |
| SEQ ID NO: | MVNTTNMTSAVVEFWERNMHQMTDGLDKPGQIRWPLAITLAIAWILVYFCIWKGVGWTGKV |
| 20 | VYFSATYPYIMLIILFFRGVTLPGAKEGILFYITPNFRKLSDSEVWLDAATQIFFSYGLGLGS |
| Human GAT-1 | LIALGSYNSFHNNVYRDSIIVCCINSCTSMFAGFVIFSIVGFMAHVTKRSIADVAASGPGLAFLA |
| isoform c | YPEAVTQLPISPLWAILFFSMLLMLGIDSQFCTVEGFITALVDEYPRLLRNRRELFIAAVCIISY |
| | LIGLSNITQGGIYVFKLFDYYSASGMSLLFLVFFECVSISWFYGVNRFYDNIQEMVGSRPCIWWK |
| | LCWSFFTPIIVAGVFIFSAVQMTPLTMGNYVFPKWGQGVGWLMALSSMVLIPGYMAYMFL |
| | TLKGSLKQRIQVMVQPSEDIVRPENGPEQPQAGSSTSKEAYI |
| SEQ ID NO: | AGAAAAACTCATCGAGCATCAAATGAAATTGCAATTTATTCATATCAGGATTATCAATAC |
| 21 | CATATTTTTGAAAAAGCCGTTTCTGTAATGAAGGAGAAAACTCACCGAGGCAGTTCCATA |
| AAV9.hu14 | GGATGGCAAGATCCTGGTATCGGTCTGCGATTCCGACTCGTCCAACATCAATACAACCTA |
| DNA | TTAATTTCCCCTCGTCAAAAATAAGGTTATCAAGTGAGAAATCACCATGAGTGACGACTG |
| sequence | AATCCGGTGAGAATGGCAAAAGTTTATGCATTTCTTTCCAGACTTGTTCAACAGGCCAGC |
| | CATTACGCTCGTCATCAAAATCACTCGCATCAACCAAACCGTTATTCATTCGTGATTGCGC |
| | CTGAGCGAGGCGAAATACGCGATCGCTGTTAAAAGGACAATTACAAACAGGAATCGAGT |
| | GCAACCGGCGCAGGAACACTGCCAGCGCATCAACAATATTTTCACCTGAATCAGGATATT |
| | CTTCTAATACCTGGAACGCTGTTTTTCCGGGGATCGCAGTGGTGAGTAACCATGCATCAT |
| | CAGGAGTACGGATAAAATGCTTGATGGTCGGAAGTGGCATAAATTCCGTCAGCCAGTTT |
| | AGTCTGACCATCTCATCTGTAACATCATTGGCAACGCTACCTTTGCCATGTTTCAGAAACA |
| | ACTCTGGCGCATCGGGCTTCCCATACAAGCGATAGATTGTCGCACCTGATTGCCCGACAT |
| | TATCGCGAGCCCATTTATACCCATATAAATCAGCATCCATGTTGGAATTTAATCGCGGCCT |
| | CGACGTTTCCCGTTGAATATGGCTCATATTCTTCCTTTTTCAATATTATTGAAGCATTTATC |
| | AGGGTTATTGTCTCATGAGCGGATACATATTTGAATGTATTTAGAAAAATAAACAAATAG |
| | GGGTCAGTGTTACAACCAATTAACCAATTCTGAACATTATCGCGAGCCCATTTATACCTG |
| | AATATGGCTCATAACACCCCTTGTTTGCCTGGCGGCAGTAGCGCGGTGGTCCCACCTGAC |
| | CCCATGCCGAACTCAGAAGTGAAACGCCGTAGCGCCGATGGTAGTGTGGGGACTCCCCA |
| | TGCGAGAGTAGGGAACTGCCAGGCATCAAATAAAACGAAAGGCTCAGTCGAAAGACTG |
| | GGCCTTTCGCCCGGGCTAATTAGGGGGTGTCGCCCTTCGCTGAAGTCCTGTATTAGAGGT |
| | CACGTGAGTGTTTTGCGACATTTTGCGACACCATGTGGTCACGCTGGGTATTTAAGCCCG |
| | AGTGAGCACGCAGGGTCTCCATTTTGAAGCGGGAGGTTTGAACGCGCAGCCGCCATGCC |
| | GGGGTTTTACGAGATTGTGATTAAGGTCCCCAGCGACCTTGACGAGCATCTGCCCGGCA |
| | TTTCTGACAGCTTTGTGAACTGGGTGGCCGAGAAGGAATGGGAGTTGCCGCCAGATTCT |
| | GACATGGATCTGAATCTGATTGAGCAGGCACCCCTGACCGTGGCCGAGAAGCTGCAGCG |
| | CGACTTTCTGACGGAATGGCGCCGTGTGAGTAAGGCCCCGGAGGCCCTTTTCTTTGTGCA |
| | ATTTGAGAAGGGAGAGAGCTACTTCCACATGCACGTGCTCGTGGAAACCACCGGGGTGA |
| | AATCCATGGTTTTGGGACGTTTCCTGAGTCAGATTCGCGAAAAACTGATTCAGAGAATTT |
| | ACCGCGGGATCGAGCCGACTTTGCCAAACTGGTTCGCGGTCACAAAGACCAGAAATGGC |
| | GCCGGAGGCGGGAACAAGGTGGTGGATGAGTGCTACATCCCCAATTACTTGCTCCCCAA |
| | AACCCAGCCTGAGCTCCAGTGGGCGTGGACTAATATGGAACAGTATTTAAGCGCCTGTTT |
| | GAATCTCACGGAGCGTAAACGGTTGGTGGCGCAGCATCTGACGCACGTGTCGCAGACGC |
| | AGGAGCAGAACAAAGAGAATCAGAATCCCAATTCTGATGCGCCGGTGATCAGATCAAAA |
| | ACTTCAGCCAGGTACATGGAGCTGGTCGGGTGGCTCGTGGACAAGGGGATTACCTCGG |
| | AGAAGCAGTGGATCCAGGAGGACCAGGCCTCATACATCTCCTTCAATGCGGCCTCCAAC |
| | TCGCGGTCCCAAATCAAGGCTGCCTTGGACAATGCGGGAAAGATTATGAGCCTGACTAA |
| | AACCGCCCCCGACTACCTGGTGGGCCAGCAGCCCGTGGAGGACATTTCCAGCAATCGGA |
| | TTTATAAAATTTTGGAACTAAACGGGTACGATCCCCAATATGCGGCTTCCGTCTTTCTGGG |
| | ATGGGCCACGAAAAAGTTCGGCAAGAGGAACACCATCTGGCTGTTTGGGCCTGCAACTA |
| | CCGGGAAGACCAACATCGCGGAGGCCATAGCCCACACTGTGCCCTTCTACGGGTGCGTA |
| | AACTGGACCAATGAGAACTTTCCCTTCAACGACTGTGTCGACAAGATGGTGATCTGGTG |
| | GGAGGAGGGGAAGATGACCGCCAAGGTCGTGGAGTCGGCCAAAGCCATTCTCGGAGG |
| | AAGCAAGGTGCGCGTGGACCAGAAATGCAAGTCCTCGGCCCAGATAGACCCGACTCCCG |
| | TGATCGTCACCTCCAACACCAACATGTGCGCCGTGATTGACGGGAACTCAACGACCTTCG |
| | AACACCAGCAGCCGTTGCAAGACCGGATGTTCAAATTTGAACTCACCCGCCGTCTGGATC |
| | ATGACTTTGGGAAGGTCACCAAGCAGGAAGTCAAAGACTTTTTCCGGTGGGCAAAGGAT |
| | CACGTGGTTGAGGTGGAGCATGAATTCTACGTCAAAAAGGGTGGAGCCAAGAAAAGAC |
| | CCGCCCCCAGTGACGCAGATATAAGTGAGCCCAAACGGGTGCGCGAGTCAGTTGCGCA |
| | GCCATCGACGTCAGACGCGGAAGCTTCGATCAACTACGCAGGACAGGTACCAAAACAAA |
| | TGTTCTCGTCACGTGGGCATGAATCTGATGCTGTTTCCCTGCAGACAATGCGAGAGACTG |
| | AATCAGAATTCAAATATCTGCTTCACTCACGGTGTCAAAGACTGTTTAGAGTGCTTTCCCG |
| | TGTCAGAATCTCAACCCGTTTCTGTCGTCAAAAAGGCGTATCAGAAACTGTGCTACATTC |
| | ATCACATCATGGGAAAGGTGCCAGACGCTTGCACTGCTTGCGACCTGGTCAATGTGGAC |
| | TTGGATGACTGTGTTTCTGAACAATAAATGACTTAAACCAGGTATGGCTGCCGATGGTTA |
| | TCTTCCAGATTGGCTCGAGGACAACCTTAGTGAAGGAATTCGCGAGTGGTGGGCTTTGA |
| | AACCTGGAGCCCCTCAACCCAAGGCAAATCAACAACATCAAGACAACGCTCGAGGTCTT |
| | GTGCTTCCGGGTTACAAATACCTTGGACCCGGCAACGGACTCGACAAGGGGGAGCCGGT |
| | CAACGCAGCAGACGCGGCGGCCCTCGAGCACGACAAGGCCTACGACCAGCAGCTCAAG |
| | GCCGGAGACAACCCGTACCTCAAGTACAACCACGCCGACGCCGAGTTCCAGGAGCGGCT |
| | CAAAGAAGATACGTCTTTTGGGGGCAACCTCGGGCGAGCAGTCTTCCAGGCCAAAAAGA |
| | GGCTTCTTGAACCTCTTGGTCTGGTTGAGGAAGCGGCTAAGACGGCTCCTGGAAAGAAG |
| | AGGCCTGTAGAGCAGTCTCCTCAGGAACCGGACTCCTCCGCGGGTATTGGCAAATCGGG |
| | TGCACAGCCCGCTAAAAAGAGACTCAATTTCGGTCAGACTGGCGACACAGAGTCAGTCC |
| | CAGACCCTCAACCAATCGGAGAACCTCCCGCAGCCCCCTCAGGTGTGGGATCTCTTACAA |
| | TGGCTTCAGGTGGTGGCGCACCAGTGGCAGACAATAACGAAGGTGCCGATGGAGTGGG |
| | TAGTTCCTCGGGAAATTGGCATTGCGATTCCCAATGGCTGGGGGACAGAGTCATCACCA |
| | CCAGCACCCGAACCTGGGCCCTGCCCACCTACAACAATCACCTCTACAAGCAAATCTCCA |
| | ACAGCACATCTGGAGGATCTTCAAATGACAACGCCTACTTCGGCTACAGCACCCCCTGGG |
| | GGTATTTTGACTTCAACAGATTCCACTGCCACTTCTCACCACGTGACTGGCAGCGACTCAT |
| | CAACAACAACTGGGGATTCCGGCCTAAGCGACTCAACTTCAAGCTCTTCAACATTCAGGT |
| | CAAAGAGGTTACGGACAACAATGGAGTCAAGACCATCGCCAATAACCTTACCAGCACGG |
| | TCCAGGTCTTCACGGACTCAGACTATCAGCTCCCGTACGTGCTCGGGTCGGCTCACGAGG |
| | GCTGCCTCCCGCCGTTCCCAGCGGACGTTTTCATGATTCCTCAGTACGGGTATCTGACGCT |
| | TAATGATGGAAGCCAGGCCGTGGGTCGTTCGTCCTTTTACTGCCTGGAATATTTCCCGTC |
| | GCAAATGCTAAGAACGGGTAACAACTTCCAGTTCAGCTACGAGTTTGAGAACGTACCTTT |
| | CCATAGCAGCTACGCTCACAGCCAAAGCCTGGACCGACTAATGAATCCACTCATCGACCA |
| | ATACTTGTACTATCTCTCAAAGACTATTAACGGTTCTGGACAGAATCAACAAACGCTAAA |
| | ATTCAGTGTGGCCGGACCCAGCAACATGGCTGTCCAGGGAAGAAACTACATACCTGGAC |
| | CCAGCTACCGACAACAACGTGTCTCAACCACTGTGACTCAAAACAACAACAGCGAATTTG |
| | CTTGGCCTGGAGCTTCTTCTTGGGCTCTCAATGGACGTAATAGCTTGATGAATCCTGGAC |
| | CTGCTATGGCCAGCCACAAAGAAGGAGAGGACCGTTTCTTTCCTTTGTCTGGATCTTTAA |
| | TTTTTGGCAAACAAGGAACTGGAAGAGACAACGTGGATGCGGACAAAGTCATGATAACC |
| | AACGAAGAAGAAATTAAAACTACTAACCCGGTAGCAACGGAGTCCTATGGACAAGTGGC |
| | CACAAACCACCAGAGTGCCCAAGCACAGGCGCAGACCGGCTGGGTTCAAAACCAAGGA |
| | ATACTTCCGGGTATGGTTTGGCAGGACAGAGATGTGTACCTGCAAGGACCCATTTGGGC |
| | CAAAATTCCTCACACGGACGGCAACTTTCACCCTTCTCCGCTGATGGGAGGGTTTGGAAT |
| | GAAGCACCCGCCTCCTCAGATCCTCATCAAAAACACACCTGTACCTGCGGATCCTCCAAC |
| | GGCCTTCAACAAGGACAAGCTGAACTCTTTCATCACCCAGTATTCTACTGGCCAAGTCAG |
| | CGTGGAGATCGAGTGGGAGCTGCAGAAGGAAAACAGCAAGCGCTGGAACCCGGAGAT |
| | CCAGTACACTTCCAACTATTACAAGTCTAATAATGTTGAATTTGCTGTTAATACTGAAGGT |
| | GTATATAGTGAACCCCGCCCCATTGGCACCAGATACCTGACTCGTAATCTGTAATTGCTT |
| | GTTAATCAATAAACCGTTTAATTCGTTTCAGTTGAACTTTGGTCTCTGCGCGTCAAAAGGG |
| | CGACACAAAATTTATTCTAAATGCATAATAAATACTGATAACATCTTATAGTTTGTATTAT |
| | ATTTTGTATTATCGTTGACATGTATAATTTTGATATCAAAAACTGATTTTCCCTTTATTATTT |
| | TCGAGATTTATTTTCTTAATTCTCTTTAACAAACTAGAAATATTGTATATACAAAAAATCAT |
| | AAATAATAGATGAATAGTTTAATTATAGGTGTTCATCAATCGAAAAAGCAACGTATCTTA |
| | TTTAAAGTGCGTTGCTTTTTTCTCATTTATAAGGTTAAATAATTCTCATATATCAAGCAAAG |
| | TGACAGGCGCCCTTAAATATTCTGACAAATGCTCTTTCCCTAAACTCCCCCCATAAAAAAA |
| | CCCGCCGAAGCGGGTTTTTACGTTATTTGCGGATTAACGATTACTCGTTATCAGAACCGC |
| | CCAGGGGGCCCGAGCTTAAGACTGGCCGTCGTTTTACAACACAGAAAGAGTTTGTAGAA |
| | ACGCAAAAAGGCCATCCGTCAGGGGCCTTCTGCTTAGTTTGATGCCTGGCAGTTCCCTAC |
| | TCTCGCCTTCCGCTTCCTCGCTCACTGACTCGCTGCGCTCGGTCGTTCGGCTGCGGCGAG |
| | CGGTATCAGCTCACTCAAAGGCGGTAATACGGTTATCCACAGAATCAGGGGATAACGCA |
| | GGAAAGAACATGTGAGCAAAAGGCCAGCAAAAGGCCAGGAACCGTAAAAAGGCCGCG |
| | TTGCTGGCGTTTTTCCATAGGCTCCGCCCCCCTGACGAGCATCACAAAAATCGACGCTCA |
| | AGTCAGAGGTGGCGAAACCCGACAGGACTATAAAGATACCAGGCGTTTCCCCCTGGAAG |
| | CTCCCTCGTGCGCTCTCCTGTTCCGACCCTGCCGCTTACCGGATACCTGTCCGCCTTTCTCC |
| | CTTCGGGAAGCGTGGCGCTTTCTCATAGCTCACGCTGTAGGTATCTCAGTTCGGTGTAGG |
| | TCGTTCGCTCCAAGCTGGGCTGTGTGCACGAACCCCCCGTTCAGCCCGACCGCTGCGCCT |
| | TATCCGGTAACTATCGTCTTGAGTCCAACCCGGTAAGACACGACTTATCGCCACTGGCAG |
| | CAGCCACTGGTAACAGGATTAGCAGAGCGAGGTATGTAGGCGGTGCTACAGAGTTCTTG |
| | AAGTGGTGGGCTAACTACGGCTACACTAGAAGAACAGTATTTGGTATCTGCGCTCTGCT |
| | GAAGCCAGTTACCTTCGGAAAAAGAGTTGGTAGCTCTTGATCCGGCAAACAAACCACCG |
| | CTGGTAGCGGTGGTTTTTTTGTTTGCAAGCAGCAGATTACGCGCAGAAAAAAAGGATCT |
| | CAAGAAGATCCTTTGATCTTTTCTACGGGGTCTGACGCTCAGTGGAACGACGCGCGCGTA |
| | ACTCACGTTAAGGGATTTTGGTCATGAGCTTGCGCCGTCCCGTCAAGTCAGCGTAATGCT |
| | CTGCTT |
| SEQ ID NO: | AGGAACCCCTAGTGATGGAGTTGGCCACTCCCTCTCTGCGCGCTCGCTCGCTCACTGAGG |
| 22 | CCGGGCGACCAAAGGTCGCCCGACGCCCGGGCTTTGCCCGGGCGGCCTCAGTGAGCGA |
| 5′ ITR | GCGAGCGCGC |
| SEQ ID NO: | GCGCGCTCGCTCGCTCACTGAGGCCGCCCGGGCAAAGCCCGGGCGTCGGGCGACCTTTG |
| 23 | GTCGCCCGGCCTCAGTGAGCGAGCGAGCGCGCAGAGAGGGAGTGGCCAACTCCATCAC |
| 3′ ITR | TAGGGGTTCCT |
| SEQ ID NO: | MAADGYLPDWLEDTLSEGIRQWWKLKPGPPPPKPAERHKDDSRGLVLPGYKYLGPFNGLD |
| 24 | KGEPVNEADAAALEHDKAYDRQLDSGDNPYLKYNHADAEFQERLKEDTSFGGNLGRAVFQ |
| AAV true | AKKRILEPLGLVEEPVKTAPGKKRPVEHSPAEPDSSSGTGKSGQQPARKRLNFGQTGDADSV |
| type (AAVtt) | PDPQPLGQPPAAPSGLGTNTMASGSGAPMADNNEGADGVGNSSGNWHCDSTWMGDR |
| | VITTSTRTWALPTYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQLPYVLGSAHQGCL |
| | PPFPADVFMVPQYGYLTLNNGSQAVGRSSFYCLEYFPSQMLRTGNNFTFSYTFEDVPFHSSY |
| | AHSQSLDRLMNPLIDQYLYYLSRTNTPSGTTTMSRLQFSQAGASDIRDQSRNWLPGPCYRQ |
| | QRVSKTAADNNNSDYSWTGATKYHLNGRDSLVNPGPAMASHKDDEEKYFPQSGVLIFGKQ |
| | DSGKTNVDIEKVMITDEEEIRTTNPVATEQYGSVSTNLQSGNTQAATSDVNTQGVLPGMV |
| | WQDRDVYLQGPIWAKIPHTDGHFHPSPLMGGFGLKHPPPQILIKNTPVPANPSTTFSAAKF |
| | ASFITQYSTGQVSVEIEWELQKENSKRWNPEIQYTSNYNKSVNVDFTVDTNGVYSEPRPIGTR |
| | YLTRNL |
| SEQ ID NO: | MAADGYLPDWLEDNLSEGIREWWALKPGAPQPKANQQHQDNARGLVLPGYKYLGPGNG |
| 25 | LDKGEPVNAADAAALEHDKAYDQQLKAGDNPYLKYNHADAEFQERLKEDTSFGGNLGRAV |
| AAV9 | FQAKKRLLEPLGLVEEAAKTAPGKKRPVEQSPQEPDSSAGIGKSGAQPAKKRLNFGQTGDTE |
| | SVPDPQPIGEPPAAPSGVGSLTMASGGGAPVADNNEGADGVGSSSGNWHCDSQWLGDR |
| | VITTSTRTWALPTYNNHLYKQISNSTSGGSSNDNAYFGYSTPWGYFDFNRFHCHFSPRDWQ |
| | RLINNNWGFRPKRLNFKLFNIQVKEVTDNNGVKTIANNLTSTVQVFTDSDYQLPYVLGSAHE |
| | GCLPPFPADVFMIPQYGYLTLNDGSQAVGRSSFYCLEYFPSQMLRTGNNFQFSYEFENVPFH |
| | SSYAHSQSLDRLMNPLIDQYLYYLSKTINGSGQNQQTLKFSVAGPSNMAVQGRNYIPGPSYR |
| | QQRVSTTVTQNNNSEFAWPGASSWALNGRNSLMNPGPAMASHKEGEDRFFPLSGSLIFGK |
| | QGTGRDNVDADKVMITNEEEIKTTNPVATESYGQVATNHQSAQAQAQTGWVQNQGILPG |
| | MVWQDRDVYLQGPIWAKIPHTDGNFHPSPLMGGFGMKHPPPQILIKNTPVPADPPTAFN |
| | KDKLNSFITQYSTGQVSVEIEWELQKENSKRWNPEIQYTSNYYKSNNVEFAVNTEGVYSEPRP |
| | IGTRYLTRNL |
| SEQ ID NO: | GCCCTCGGAAGACCGAGACAGCGGAGAGGTTGCGGGTGAGCTGCGCTGAGCCCAGGA |
| 26 | GCCGAGGAGTCGGGAGCGCAGTAGCGCTGAGCCCGAGCCCGAGCGGCCCCGCGTCCCG |
| Human | AGCGCATCGGAGCGGCCGAGCCGCCCGGATGCAGCGCCTGTCCCGGGCAGCGCAGCCC |
| SLC6A1 | CGGCCGCAGGATCTCACCCAGGGTGGCAGAAGGAGGCCTTCTGGAGCTGACCCACCCCC |
| transcript | GACGACCATCAGGGTGCCCTTGAGCCGCAAAACTGCTGTCCACGTGGACCGGGGGTGAC |
| variant 2 | ATCGCACGTCCATCTGCCAGGACCCCTGCGTCCAAATTCCGAGACATGGCGACCAACGG |
| (isoform a) | CAGCAAGGTGGCCGACGGGCAGATCTCCACCGAGGTCAGCGAGGCCCCTGTGGCCAAT |
| | GACAAGCCCAAAACCTTGGTGGTCAAGGTGCAGAAGAAGGCGGCAGACCTCCCCGACC |
| | GGGACACGTGGAAGGGCCGCTTCGACTTCCTCATGTCCTGTGTGGGCTATGCCATCGGC |
| | CTGGGCAACGTCTGGAGGTTCCCCTATCTCTGCGGGAAAAATGGTGGGGGAGCCTTCCT |
| | GATCCCCTATTTCCTGACACTCATCTTTGCGGGGGTCCCACTCTTCCTGCTGGAGTGCTCC |
| | CTGGGCCAGTACACCTCCATCGGGGGGCTAGGGGTATGGAAGCTGGCTCCTATGTTCAA |
| | GGGCGTGGGCCTTGCGGCTGCTGTGCTATCATTCTGGCTGAACATCTACTACATCGTCAT |
| | CATCTCCTGGGCCATTTACTACCTGTACAACTCCTTCACCACGACACTGCCGTGGAAACAG |
| | TGCGACAACCCCTGGAACACAGACCGCTGCTTCTCCAACTACAGCATGGTCAACACTACC |
| | AACATGACCAGCGCTGTGGTGGAGTTCTGGGAGCGCAACATGCATCAGATGACGGACG |
| | GGCTGGATAAGCCAGGTCAGATCCGCTGGCCACTGGCCATCACGCTGGCCATCGCCTGG |
| | ATCCTTGTGTATTTCTGTATCTGGAAGGGTGTTGGCTGGACTGGAAAGGTGGTCTACTTT |
| | TCAGCCACATACCCCTACATCATGCTGATCATCCTGTTCTTCCGTGGAGTGACGCTGCCCG |
| | GGGCCAAGGAGGGCATCCTCTTCTACATCACACCCAACTTCCGCAAGCTGTCTGACTCCG |
| | AGGTGTGGCTGGATGCGGCAACCCAGATCTTCTTCTCATACGGGCTGGGCCTGGGGTCC |
| | CTGATCGCTCTCGGGAGCTACAACTCTTTCCACAACAATGTCTACAGGGACTCCATCATC |
| | GTCTGCTGCATCAATTCGTGCACCAGCATGTTCGCAGGATTCGTCATCTTCTCCATCGTGG |
| | GCTTCATGGCCCATGTCACCAAGAGGTCCATTGCTGATGTGGCGGCCTCAGGCCCCGGG |
| | CTGGCGTTCCTGGCATACCCAGAGGCGGTGACCCAGCTGCCTATCTCCCCACTCTGGGCC |
| | ATCCTCTTCTTCTCCATGCTGTTGATGCTGGGCATTGACAGCCAGTTCTGCACTGTGGAG |
| | GGCTTCATCACAGCCCTGGTGGATGAGTACCCCAGGCTCCTCCGCAACCGCAGAGAGCT |
| | CTTCATTGCTGCTGTCTGCATCATCTCCTACCTGATCGGTCTCTCTAACATCACTCAGGGG |
| | GGTATTTATGTCTTCAAACTCTTTGACTACTACTCTGCCAGTGGCATGAGCCTGCTGTTCC |
| | TCGTGTTCTTTGAATGTGTCTCTATTTCCTGGTTTTACGGTGTCAACCGATTCTATGACAAT |
| | ATCCAAGAGATGGTTGGATCCAGGCCCTGCATCTGGTGGAAACTCTGCTGGTCTTTCTTC |
| | ACACCAATCATTGTGGCGGGCGTGTTCATTTTCAGTGCTGTGCAGATGACGCCACTCACC |
| | ATGGGAAACTATGTTTTCCCCAAGTGGGGCCAGGGTGTGGGCTGGCTGATGGCTCTGTC |
| | TTCCATGGTCCTCATCCCCGGGTACATGGCCTACATGTTCCTCACCTTAAAGGGCTCCCTG |
| | AAGCAGCGCATCCAAGTCATGGTCCAGCCCAGCGAAGACATCGTTCGCCCAGAGAATGG |
| | TCCTGAGCAGCCCCAGGCGGGCAGCTCCACCAGCAAGGAGGCCTACATCTAGGGTGGG |
| | GGCCACTCACCGACCCGACACTCTCACCCCCCGACCTGGCTGAGTGCGACCACCACTTGA |
| | TGTCTGAGGATACCTTCCATCTCAACCTACCTCGAGTGGCGAGTCCAGACACCATCACCA |
| | CGCAGAGAGGGGAGGTGGGAGGACAGTTAGACCCCTGGGTGGGCCCTGCCGTGGGCA |
| | AGGATACCCGGTGGCTTCTGGCACCTGGCGGGCTGGTGACCTTTTTAATCCAGGCCCCAT |
| | GTGTGATGCAGGAAGACCACATGCGCTCCTGGCTTTTAAACCTGTTCCTGACTGTTCTCTT |
| | ACTGCCGAAACCCTTGACTGTTATCTCGGACTTTGCAGGAGTTCCTTTCCCTCCGAACGCT |
| | GCTCCATGCACAGGAAAAGGGCATTTTGTACAATGGGGACTTCCCGGGAACGCTTGCTC |
| | TTAAGTACCAGAAGCCGGCGGAGCTCTGGCTTTCGTGTTTTTGGTTTTCTCCTTCCCAAGG |
| | CAGCTGGATTGAAAAAACAAAACAAAACAAAAAAACCCAGGGGCGTCAGTCGATATTCC |
| | CAGGGCCGCTTCTCCTGCAGTCTGTGGAGCGTCCTTGTCCCCGCCGCCGGAATGAATGA |
| | GCATTCTGCAGCCCGATGTCCCTGTCCCCTCCTCGCCGGGCCATTCTGATTGGACCTGGCC |
| | CAGTGCAATCTGTCCAGACAAGCCCTGCTTGCTGGAAAACTGCCACAAGCACAATTGATC |
| | TCTTTTTATCGCCATTCCAGGGGCCTCAGGTCCTACTGGGGAAACTTCCTATACCGGAGCT |
| | CCAGTTTCTCTTAAGCTGCCCAATTTCACAGAGTACAAAATAGTTGTAGGGGAAATCAAG |
| | GTGAAGGATCTGTCCGACAGTCAAGACGGATCCACAGTAATCTTTCGGTCTCCTTAAACT |
| | ACCACCCTCGCTGCCACCCACCCCAAGCTGCTGCCGCCTCACCTTCCTTGAAATTTCTCAG |
| | CGGGAGTCTCCTCACTGCCACTAAAATCCACCCAGCCCACTAACTGAGGAGCTAGTGT |
| | TAATCCAGAGAACCCCCCGCAATGTGCTTCCGAGATTCAGACTGCTTCATTGGGAAGTAT |
| | GATTTGTTCCTTTCTGGAATTGGGCTCCGTGGTGGCGGCGGCACTTCAAGCAAAGACAGT |
| | TTCTTGCAAGCTCCAGTAGCTCCGCGTGTCTCATTTGCCAGGAAGATGGGTTCCCACGTA |
| | GCAAATCGTACATTGTGCCCTGTAGCTCCTTAGCTAGTTAGCTCACAAGCCGTGTTTTATG |
| | ACTAATCCTTAATAACTATGGTAAATAACTGTGACTGTGGGGTTTTTAATCTCTTGTCATT |
| | CTCATCCAAAAGTGACCAGCATACCAGTTCTTGCAATAAGATATTACCCTCAGAATATTAA |
| | GCACATTATTGTAGAGAAAAAAAAATATGTGTACACATATGAACGCACAACATGCACATT |
| | CATCCTCACATGTGGCACGTAAGGTCTCATTTGATATTGTGTAGGAAATCTGAAGCCTTTT |
| | CCTGAGGTCATCTGTAAAATAGTCTCATTGCCAAGGCATCCCCAGTGCCAGCTGGTGAAT |
| | CCATGATCAAAATGCATACGTATTGTTAAATGATAAGGTTTAGAATGACAGGAACCCATC |
| | ACTGTGTCTCATGGTCCCACTTCCCCATCTGTGTGTGAATTCCTTTAGACTAAGGGCAGGA |
| | AGACTTCCAGCTTTCTCTTTGTTCTTCAATGTGAAACTGAGACCAAGTCTCTCTAAGACAA |
| | ATGCAGTGTATTTAATGTTTGTAAGCAATTCTAAGTGAGATGTTTGGCAAGAAATCCCCT |
| | AACTGATTTCCATCCAAACCTACCTTATAGAGCACAATATTAAGTGTTGTACAATTACTGT |
| | GAGAACTGTGAATATGTGTAACTTTTTTTTAGTATTTGCCCGGGGGGAAAAAGATATTGT |
| | ATTATCATATATGCTTTTTTGCAATAAGGATTTATTCTCAGAACACCAAGTAAATCTATCTC |
| | TATATAAAAAATATATGTAATATATACATATTCAAAGTATATACAGAGCCTGTTTTAAAAA |
| | ATACAGTATTATTTAGTAAAATTATCTGTTCTATGGACCAAATGTAAAATATTTATAAATG |
| | AAGATGCATTTTAAATGTCTATAAATGGTGTCATAACTAGAGCACGGGCGTTATGTAAGT |
| | TTCTAAGAATTTAGAGGATAAATAATAAAGGTTCTATGATATACAA |
| SEQ ID NO: | GCCCTCGGAAGACCGAGACAGCGGAGAGGTTGCGGGTGAGCTGCGCTGAGCCCAGGA |
| 27 | GCCGAGGAGTCGGGAGCGCAGTAGCGCTGAGCCCGAGCCCGAGCGGCCCCGCGTCCCG |
| Human | AGCGCATCGGAGCGGCCGAGCCGCCCGGATGCAGCGCCTGTCCCGGGCAGCGCAGCCC |
| SLC6A1 | CGGCCGCAGGATCTCACCCAGGGTGGCAGAAGGAGGCCTTCTGGAGCTGACCCACCCCC |
| transcript | GACGACCATCAGGGTGCCCTTGAGCCGCAAAACTGCTGTCCACGTGGACCGGGGGTGAC |
| variant 3 | ATCGCACGTCCATCTGCCAGGACCCCTGCGTCCAAATTCCGAGACATGGCGACCAACGG |
| (isoform b) | CAGCAAGGTGGCCGACGGGCAGATCTCCACCGAGGAGCCTTCCTGATCCCCTATTTCCTG |
| | ACACTCATCTTTGCGGGGGTCCCACTCTTCCTGCTGGAGTGCTCCCTGGGCCAGTACACC |
| | TCCATCGGGGGGCTAGGGGTATGGAAGCTGGCTCCTATGTTCAAGGGCGTGGGCCTTGC |
| | GGCTGCTGTGCTATCATTCTGGCTGAACATCTACTACATCGTCATCATCTCCTGGGCCATT |
| | TACTACCTGTACAACTCCTTCACCACGACACTGCCGTGGAAACAGTGCGACAACCCCTGG |
| | AACACAGACCGCTGCTTCTCCAACTACAGCATGGTCAACACTACCAACATGACCAGCGCT |
| | GTGGTGGAGTTCTGGGAGCGCAACATGCATCAGATGACGGACGGGCTGGATAAGCCAG |
| | GTCAGATCCGCTGGCCACTGGCCATCACGCTGGCCATCGCCTGGATCCTTGTGTATTTCT |
| | GTATCTGGAAGGGTGTTGGCTGGACTGGAAAGGTGGTCTACTTTTCAGCCACATACCCCT |
| | ACATCATGCTGATCATCCTGTTCTTCCGTGGAGTGACGCTGCCCGGGGCCAAGGAGGGC |
| | ATCCTCTTCTACATCACACCCAACTTCCGCAAGCTGTCTGACTCCGAGGTGTGGCTGGAT |
| | GCGGCAACCCAGATCTTCTTCTCATACGGGCTGGGCCTGGGGTCCCTGATCGCTCTCGGG |
| | AGCTACAACTCTTTCCACAACAATGTCTACAGGGACTCCATCATCGTCTGCTGCATCAATT |
| | CGTGCACCAGCATGTTCGCAGGATTCGTCATCTTCTCCATCGTGGGCTTCATGGCCCATGT |
| | CACCAAGAGGTCCATTGCTGATGTGGCGGCCTCAGGCCCCGGGCTGGCGTTCCTGGCAT |
| | ACCCAGAGGCGGTGACCCAGCTGCCTATCTCCCCACTCTGGGCCATCCTCTTCTTCTCCAT |
| | GCTGTTGATGCTGGGCATTGACAGCCAGTTCTGCACTGTGGAGGGCTTCATCACAGCCCT |
| | GGTGGATGAGTACCCCAGGCTCCTCCGCAACCGCAGAGAGCTCTTCATTGCTGCTGTCTG |
| | CATCATCTCCTACCTGATCGGTCTCTCTAACATCACTCAGGGGGGTATTTATGTCTTCAAA |
| | CTCTTTGACTACTACTCTGCCAGTGGCATGAGCCTGCTGTTCCTCGTGTTCTTTGAATGTG |
| | TCTCTATTTCCTGGTTTTACGGTGTCAACCGATTCTATGACAATATCCAAGAGATGGTTGG |
| | ATCCAGGCCCTGCATCTGGTGGAAACTCTGCTGGTCTTTCTTCACACCAATCATTGTGGCG |
| | GGCGTGTTCATTTTCAGTGCTGTGCAGATGACGCCACTCACCATGGGAAACTATGTTTTC |
| | CCCAAGTGGGGCCAGGGTGTGGGCTGGCTGATGGCTCTGTCTTCCATGGTCCTCATCCCC |
| | GGGTACATGGCCTACATGTTCCTCACCTTAAAGGGCTCCCTGAAGCAGCGCATCCAAGTC |
| | ATGGTCCAGCCCAGCGAAGACATCGTTCGCCCAGAGAATGGTCCTGAGCAGCCCCAGGC |
| | GGGCAGCTCCACCAGCAAGGAGGCCTACATCTAGGGTGGGGGCCACTCACCGACCCGA |
| | CACTCTCACCCCCCGACCTGGCTGAGTGCGACCACCACTTGATGTCTGAGGATACCTTCC |
| | ATCTCAACCTACCTCGAGTGGCGAGTCCAGACACCATCACCACGCAGAGAGGGGAGGTG |
| | GGAGGACAGTTAGACCCCTGGGTGGGCCCTGCCGTGGGCAAGGATACCCGGTGGCTTC |
| | TGGCACCTGGCGGGCTGGTGACCTTITTAATCCAGGCCCCATCAGCATCCCACGATCGGC |
| | CTTGGTAACCGCCGCGGTAGATCATTTTTATCCCGCCAGGGAGTGTGATGCAGGAAGAC |
| | CACATGCGCTCCTGGCTTTTAAACCTGTTCCTGACTGTTCTCTTACTGCCGAAACCCTTGA |
| | CTGTTATCTCGGACTTTGCAGGAGTTCCTTTCCCTCCGAACGCTGCTCCATGCACAGGAAA |
| | AGGGCATTTTGTACAATGGGGACTTCCCGGGAACGCTTGCTCTTAAGTACCAGAAGCCG |
| | GCGGAGCTCTGGCTTTCGTGTTTTTGGTTTTCTCCTTCCCAAGGCAGCTGGATTGAAAAA |
| | ACAAAACAAAACAAAAAAACCCAGGGGCGTCAGTCGATATTCCCAGGGCCGCTTCTCCT |
| | GCAGTCTGTGGAGCGTCCTTGTCCCCGCCGCCGGAATGAATGAGCATTCTGCAGCCCGA |
| | TGTCCCTGTCCCCTCCTCGCCGGGCCATTCTGATTGGACCTGGCCCAGTGCAATCTGTCCA |
| | GACAAGCCCTGCTTGCTGGAAAACTGCCACAAGCACAATTGATCTCTTTTTATCGCCATTC |
| | CAGGGGCCTCAGGTCCTACTGGGGAAACTTCCTATACCGGAGCTCCAGTTTCTCTTAAGC |
| | TGCCCAATTTCACAGAGTACAAAATAGTTGTAGGGGAAATCAAGGTGAAGGATCTGTCC |
| | GACAGTCAAGACGGATCCACAGTAATCTTTCGGTCTCCTTAAACTACCACCCTCGCTGCC |
| | ACCCACCCCAAGCTGCTGCCGCCTCACCTTCCTTGAAATTTCTCAGCGGGAGTCTCCTCAC |
| | TGCCACTAAAATCCACCCAGCCCACTAACTGAGGAGCTAGTGTTAATCCAGAGAACCCCC |
| | CGCAATGTGCTTCCGAGATTCAGACTGCTTCATTGGGAAGTATGATTTGTTCCTTTCTGGA |
| | ATTGGGCTCCGTGGTGGCGGCGGCACTTCAAGCAAAGACAGTTTCTTGCAAGCTCCAGT |
| | AGCTCCGCGTGTCTCATTTGCCAGGAAGATGGGTTCCCACGTAGCAAATCGTACATTGTG |
| | CCCTGTAGCTCCTTAGCTAGTTAGCTCACAAGCCGTGTTTTATGACTAATCCTTAATAACT |
| | ATGGTAAATAACTGTGACTGTGGGGTTTTTAATCTCTTGTCATTCTCATCCAAAAGTGACC |
| | AGCATACCAGTTCTTGCAATAAGATATTACCCTCAGAATATTAAGCACATTATTGTAGAG |
| | AAAAAAAAATATGTGTACACATATGAACGCACAACATGCACATTCATCCTCACATGTGGC |
| | ACGTAAGGTCTCATTTGATATTGTGTAGGAAATCTGAAGCCTTTTCCTGAGGTCATCTGTA |
| | AAATAGTCTCATTGCCAAGGCATCCCCAGTGCCAGCTGGTGAATCCATGATCAAAATGCA |
| | TACGTATTGTTAAATGATAAGGTTTAGAATGACAGGAACCCATCACTGTGTCTCATGGTC |
| | CCACTTCCCCATCTGTGTGTGAATTCCTTTAGACTAAGGGCAGGAAGACTTCCAGCTTTC |
| | TCTTTGTTCTTCAATGTGAAACTGAGACCAAGTCTCTCTAAGACAAATGCAGTGTATTTAA |
| | TGTTTGTAAGCAATTCTAAGTGAGATGTTTGGCAAGAAATCCCCTAACTGATTTCCATCCA |
| | AACCTACCTTATAGAGCACAATATTAAGTGTTGTACAATTACTGTGAGAACTGTGAATAT |
| | GTGTAACTTTTTTTTAGTATTTGCCCGGGGGGAAAAAGATATTGTATTATCATATATGCTT |
| | TTTTGCAATAAGGATTTATTCTCAGAACACCAAGTAAATCTATCTCTATATAAAAAATATA |
| | TGTAATATATACATATTCAAAGTATATACAGAGCCTGTITTAAAAAATACAGTATTATTTA |
| | GTAAAATTATCTGTTCTATGGACCAAATGTAAAATATTTATAAATGAAGATGCATTTTAAA |
| | TGTCTATAAATGGTGTCATAACTAGAGCACGGGCGTTATGTAAGTTTCTAAGAATTTAGA |
| | GGATAAATAATAAAGGTTCTATGATATACAA |
| SEQ ID NO: | GCCCTCGGAAGACCGAGACAGCGGAGAGGTTGCGGGTGAGCTGCGCTGAGCCCAGGA |
| 28 | GCCGAGGAGTCGGGAGCGCAGTAGCGCTGAGCCCGAGCCCGAGCGGCCCCGCGTCCCG |
| Human | AGCGCATCGGAGCGGCCGAGCCGCCCGGATGCAGCGCCTGTCCCGGGCAGCGCAGCCC |
| SLC6A1 | CGGCCGCAGGATCTCACCCAGGGTGGCAGAAGGAGGCCTTCTGGAGCTGACCCACCCCC |
| transcript | GACGACCATCAGGGTGAGGCAACTCCAAGGTCCTACTCTCTTTCTGTGCCTGTTACCCAC |
| variant 4 | CCCGTCCTCCTAGGGTGCCCTTGAGCCGCAAAACTGCTGTCCACGTGGACCGGGGGTG |
| (isoform c) | ACATCGCACGTCCATCTGCCAGGACCCCTGCGTCCAAATTCCGAGACATGGCGACCAACG |
| | GCAGCAAGGTGGCCGACGGGCAGATCTCCACCGAGGCGTGGGCCTTGCGGCTGCTGTG |
| | CTATCATTCTGGCTGAACATCTACTACATCGTCATCATCTCCTGGGCCATTTACTACCTGTA |
| | CAACTCCTTCACCACGACACTGCCGTGGAAACAGTGCGACAACCCCTGGAACACAGACC |
| | GCTGCTTCTCCAACTACAGCATGGTCAACACTACCAACATGACCAGCGCTGTGGTGGAGT |
| | TCTGGGAGCGCAACATGCATCAGATGACGGACGGGCTGGATAAGCCAGGTCAGATCCG |
| | CTGGCCACTGGCCATCACGCTGGCCATCGCCTGGATCCTTGTGTATTTCTGTATCTGGAA |
| | GGGTGTTGGCTGGACTGGAAAGGTGGTCTACTTTTCAGCCACATACCCCTACATCATGCT |
| | GATCATCCTGTTCTTCCGTGGAGTGACGCTGCCCGGGGCCAAGGAGGGCATCCTCTTCTA |
| | CATCACACCCAACTTCCGCAAGCTGTCTGACTCCGAGGTGTGGCTGGATGCGGCAACCCA |
| | GATCTTCTTCTCATACGGGCTGGGCCTGGGGTCCCTGATCGCTCTCGGGAGCTACAACTC |
| | TTTCCACAACAATGTCTACAGGGACTCCATCATCGTCTGCTGCATCAATTCGTGCACCAGC |
| | ATGTTCGCAGGATTCGTCATCTTCTCCATCGTGGGCTTCATGGCCCATGTCACCAAGAGG |
| | TCCATTGCTGATGTGGCGGCCTCAGGCCCCGGGCTGGCGTTCCTGGCATACCCAGAGGC |
| | GGTGACCCAGCTGCCTATCTCCCCACTCTGGGCCATCCTCTTCTTCTCCATGCTGTTGATG |
| | CTGGGCATTGACAGCCAGTTCTGCACTGTGGAGGGCTTCATCACAGCCCTGGTGGATGA |
| | GTACCCCAGGCTCCTCCGCAACCGCAGAGAGCTCTTCATTGCTGCTGTCTGCATCATCTCC |
| | TACCTGATCGGTCTCTCTAACATCACTCAGGGGGGTATTTATGTCTTCAAACTCTTTGACT |
| | ACTACTCTGCCAGTGGCATGAGCCTGCTGTTCCTCGTGTTCTTTGAATGTGTCTCTATTTC |
| | CTGGTTTTACGGTGTCAACCGATTCTATGACAATATCCAAGAGATGGTTGGATCCAGGCC |
| | CTGCATCTGGTGGAAACTCTGCTGGTCTTTCTTCACACCAATCATTGTGGCGGGCGTGTT |
| | CATTTTCAGTGCTGTGCAGATGACGCCACTCACCATGGGAAACTATGTTTTCCCCAAGTG |
| | GGGCCAGGGTGTGGGCTGGCTGATGGCTCTGTCTTCCATGGTCCTCATCCCCGGGTACA |
| | TGGCCTACATGTTCCTCACCTTAAAGGGCTCCCTGAAGCAGCGCATCCAAGTCATGGTCC |
| | AGCCCAGCGAAGACATCGTTCGCCCAGAGAATGGTCCTGAGCAGCCCCAGGCGGGCAG |
| | CTCCACCAGCAAGGAGGCCTACATCTAGGGTGGGGGCCACTCACCGACCCGACACTCTC |
| | ACCCCCCGACCTGGCTGAGTGCGACCACCACTTGATGTCTGAGGATACCTTCCATCTCAA |
| | CCTACCTCGAGTGGCGAGTCCAGACACCATCACCACGCAGAGAGGGGAGGTGGGAGGA |
| | CAGTTAGACCCCTGGGTGGGCCCTGCCGTGGGCAAGGATACCCGGTGGCTTCTGGCACC |
| | TGGCGGGCTGGTGACCTTTTTAATCCAGGCCCCATCAGCATCCCACGATCGGCCTTGGTA |
| | ACCGCCGCGGTAGATCATTTTTATCCCGCCAGGGAGTGTGATGCAGGAAGACCACATGC |
| | GCTCCTGGCTTTTAAACCTGTTCCTGACTGTTCTCTTACTGCCGAAACCCTTGACTGTTATC |
| | TCGGACTTTGCAGGAGTTCCTTTCCCTCCGAACGCTGCTCCATGCACAGGAAAAGGGCAT |
| | TTTGTACAATGGGGACTTCCCGGGAACGCTTGCTCTTAAGTACCAGAAGCCGGCGGAGC |
| | TCTGGCTTTCGTGTTTTTGGTTTTCTCCTTCCCAAGGCAGCTGGATTGAAAAAACAAAACA |
| | AAACAAAAAAACCCAGGGGCGTCAGTCGATATTCCCAGGGCCGCTTCTCCTGCAGTCTGT |
| | GGAGCGTCCTTGTCCCCGCCGCCGGAATGAATGAGCATTCTGCAGCCCGATGTCCCTGTC |
| | CCCTCCTCGCCGGGCCATTCTGATTGGACCTGGCCCAGTGCAATCTGTCCAGACAAGCCC |
| | TGCTTGCTGGAAAACTGCCACAAGCACAATTGATCTCTTTTTATCGCCATTCCAGGGGCCT |
| | CAGGTCCTACTGGGGAAACTTCCTATACCGGAGCTCCAGTTTCTCTTAAGCTGCCCAATTT |
| | CACAGAGTACAAAATAGTTGTAGGGGAAATCAAGGTGAAGGATCTGTCCGACAGTCAA |
| | GACGGATCCACAGTAATCTTTCGGTCTCCTTAAACTACCACCCTCGCTGCCACCCACCCCA |
| | AGCTGCTGCCGCCTCACCTTCCTTGAAATTTCTCAGCGGGAGTCTCCTCACTGCCACTAAA |
| | ATCCACCCAGCCCACTAACTGAGGAGCTAGTGTTAATCCAGAGAACCCCCCGCAATGTGC |
| | TTCCGAGATTCAGACTGCTTCATTGGGAAGTATGATTTGTTCCTTTCTGGAATTGGGCTCC |
| | GTGGTGGCGGCGGCACTTCAAGCAAAGACAGTTTCTTGCAAGCTCCAGTAGCTCCGCGT |
| | GTCTCATTTGCCAGGAAGATGGGTTCCCACGTAGCAAATCGTACATTGTGCCCTGTAGCT |
| | CCTTAGCTAGTTAGCTCACAAGCCGTGTTTTATGACTAATCCTTAATAACTATGGTAAATA |
| | ACTGTGACTGTGGGGTTTTTAATCTCTTGTCATTCTCATCCAAAAGTGACCAGCATACCAG |
| | TTCTTGCAATAAGATATTACCCTCAGAATATTAAGCACATTATTGTAGAGAAAAAAAAAT |
| | ATGTGTACACATATGAACGCACAACATGCACATTCATCCTCACATGTGGCACGTAAGGTC |
| | TCATTTGATATTGTGTAGGAAATCTGAAGCCTTTTCCTGAGGTCATCTGTAAAATAGTCTC |
| | ATTGCCAAGGCATCCCCAGTGCCAGCTGGTGAATCCATGATCAAAATGCATACGTATTGT |
| | TAAATGATAAGGTTTAGAATGACAGGAACCCATCACTGTGTCTCATGGTCCCACTTCCCC |
| | ATCTGTGTGTGAATTCCTTTAGACTAAGGGCAGGAAGACTTCCAGCTTTCTCTTTGTTCTT |
| | CAATGTGAAACTGAGACCAAGTCTCTCTAAGACAAATGCAGTGTATTTAATGTTTGTAAG |
| | CAATTCTAAGTGAGATGTTTGGCAAGAAATCCCCTAACTGATTTCCATCCAAACCTACCTT |
| | ATAGAGCACAATATTAAGTGTTGTACAATTACTGTGAGAACTGTGAATATGTGTAACTTT |
| | TTTTTAGTATTTGCCCGGGGGGAAAAAGATATTGTATTATCATATATGCTTTTTTGCAATA |
| | AGGATTTATTCTCAGAACACCAAGTAAATCTATCTCTATATAAAAAATATATGTAATATAT |
| | ACATATTCAAAGTATATACAGAGCCTGTTTTAAAAAATACAGTATTATTTAGTAAAATTAT |
| | CTGTTCTATGGACCAAATGTAAAATATTTATAAATGAAGATGCATTTTAAATGTCTATAAA |
| | TGGTGTCATAACTAGAGCACGGGCGTTATGTAAGTTTCTAAGAATTTAGAGGATAAATA |
| | ATAAAGGTTCTATGATATACAA |
| SEQ ID NO: | GCCCTCGGAAGACCGAGACAGCGGAGAGGTTGCGGGTGAGCTGCGCTGAGCCCAGGA |
| 29 | GCCGAGGAGTCGGGAGCGCAGTAGCGCTGAGCCCGAGCCCGAGCGGCCCCGCGTCCCG |
| Human | AGCGCATCGGAGCGGCCGAGCCGCCCGGATGCAGCGCCTGTCCCGGGCAGCGCAGCCC |
| SLC6A1 | CGGCCGCAGGATCTCACCCAGGGTGGCAGAAGGAGGCCTTCTGGAGCTGACCCACCCCC |
| transcript | GACGACCATCAGGGTGCCCTTGAGCCGCAAAACTGCTGTCCACGTGGACCGGGGGTGAC |
| variant 5 | ATCGCACGTCCATCTGCCAGGACCCCTGCGTCCAAATTCCGAGACATGGCGACCAACGG |
| (isoform c) | CAGCAAGGTGGCCGACGGGCAGATCTCCACCGAGGCGTGGGCCTTGCGGCTGCTGTGC |
| | TATCATTCTGGCTGAACATCTACTACATCGTCATCATCTCCTGGGCCATTTACTACCTGTAC |
| | AACTCCTTCACCACGACACTGCCGTGGAAACAGTGCGACAACCCCTGGAACACAGACCG |
| | CTGCTTCTCCAACTACAGCATGGTCAACACTACCAACATGACCAGCGCTGTGGTGGAGTT |
| | CTGGGAGCGCAACATGCATCAGATGACGGACGGGCTGGATAAGCCAGGTCAGATCCGC |
| | TGGCCACTGGCCATCACGCTGGCCATCGCCTGGATCCTTGTGTATTTCTGTATCTGGAAG |
| | GGTGTTGGCTGGACTGGAAAGGTGGTCTACTTTTCAGCCACATACCCCTACATCATGCTG |
| | ATCATCCTGTTCTTCCGTGGAGTGACGCTGCCCGGGGCCAAGGAGGGCATCCTCTTCTAC |
| | ATCACACCCAACTTCCGCAAGCTGTCTGACTCCGAGGTGTGGCTGGATGCGGCAACCCA |
| | GATCTTCTTCTCATACGGGCTGGGCCTGGGGTCCCTGATCGCTCTCGGGAGCTACAACTC |
| | TTTCCACAACAATGTCTACAGGGACTCCATCATCGTCTGCTGCATCAATTCGTGCACCAGC |
| | ATGTTCGCAGGATTCGTCATCTTCTCCATCGTGGGCTTCATGGCCCATGTCACCAAGAGG |
| | TCCATTGCTGATGTGGCGGCCTCAGGCCCCGGGCTGGCGTTCCTGGCATACCCAGAGGC |
| | GGTGACCCAGCTGCCTATCTCCCCACTCTGGGCCATCCTCTTCTTCTCCATGCTGTTGATG |
| | CTGGGCATTGACAGCCAGTTCTGCACTGTGGAGGGCTTCATCACAGCCCTGGTGGATGA |
| | GTACCCCAGGCTCCTCCGCAACCGCAGAGAGCTCTTCATTGCTGCTGTCTGCATCATCTCC |
| | TACCTGATCGGTCTCTCTAACATCACTCAGGGGGGTATTTATGTCTTCAAACTCTTTGACT |
| | ACTACTCTGCCAGTGGCATGAGCCTGCTGTTCCTCGTGTTCTTTGAATGTGTCTCTATTTC |
| | CTGGTTTTACGGTGTCAACCGATTCTATGACAATATCCAAGAGATGGTTGGATCCAGGCC |
| | CTGCATCTGGTGGAAACTCTGCTGGTCTTTCTTCACACCAATCATTGTGGCGGGCGTGTT |
| | CATTTTCAGTGCTGTGCAGATGACGCCACTCACCATGGGAAACTATGTTTTCCCCAAGTG |
| | GGGCCAGGGTGTGGGCTGGCTGATGGCTCTGTCTTCCATGGTCCTCATCCCCGGGTACA |
| | TGGCCTACATGTTCCTCACCTTAAAGGGCTCCCTGAAGCAGCGCATCCAAGTCATGGTCC |
| | AGCCCAGCGAAGACATCGTTCGCCCAGAGAATGGTCCTGAGCAGCCCCAGGCGGGCAG |
| | CTCCACCAGCAAGGAGGCCTACATCTAGGGTGGGGGCCACTCACCGACCCGACACTCTC |
| | ACCCCCCGACCTGGCTGAGTGCGACCACCACTTGATGTCTGAGGATACCTTCCATCTCAA |
| | CCTACCTCGAGTGGCGAGTCCAGACACCATCACCACGCAGAGAGGGGAGGTGGGAGGA |
| | CAGTTAGACCCCTGGGTGGGCCCTGCCGTGGGCAAGGATACCCGGTGGCTTCTGGCACC |
| | TGGCGGGCTGGTGACCTTTTTAATCCAGGCCCCATCAGCATCCCACGATCGGCCTTGGTA |
| | ACCGCCGCGGTAGATCATTTTTATCCCGCCAGGGAGTGTGATGCAGGAAGACCACATGC |
| | GCTCCTGGCTTTTAAACCTGTTCCTGACTGTTCTCTTACTGCCGAAACCCTTGACTGTTATC |
| | TCGGACTTTGCAGGAGTTCCTTTCCCTCCGAACGCTGCTCCATGCACAGGAAAAGGGCAT |
| | TTTGTACAATGGGGACTTCCCGGGAACGCTTGCTCTTAAGTACCAGA |
| | AGCCGGCGGAGCTCTGGCTTTCGTGTTTTTGGTTTTCTCCTTCCCAAGGCAGCTGGATTG |
| | AAAAAACAAAACAAAACAAAAAAACCCAGGGGCGTCAGTCGATATTCCCAGGGCCGCTT |
| | CTCCTGCAGTCTGTGGAGCGTCCTTGTCCCCGCCGCCGGAATGAATGAGCATTCTGCAGC |
| | CCGATGTCCCTGTCCCCTCCTCGCCGGGCCATTCTGATTGGACCTGGCCCAGTGCAATCT |
| | GTCCAGACAAGCCCTGCTTGCTGGAAAACTGCCACAAGCACAATTGATCTCTTTTTATCG |
| | CCATTCCAGGGGCCTCAGGTCCTACTGGGGAAACTTCCTATACCGGAGCTCCAGTTTCTC |
| | TTAAGCTGCCCAATTTCACAGAGTACAAAATAGTTGTAGGGGAAATCAAGGTGAAGGAT |
| | CTGTCCGACAGTCAAGACGGATCCACAGTAATCTTTCGGTCTCCTTAAACTACCACCCTCG |
| | CTGCCACCCACCCCAAGCTGCTGCCGCCTCACCTTCCTTGAAATTTCTCAGCGGGAGTCTC |
| | CTCACTGCCACTAAAATCCACCCAGCCCACTAACTGAGGAGCTAGTGTTAATCCAGAGAA |
| | CCCCCCGCAATGTGCTTCCGAGATTCAGACTGCTTCATTGGGAAGTATGATTTGTTCCTTT |
| | CTGGAATTGGGCTCCGTGGTGGCGGCGGCACTTCAAGCAAAGACAGTTTCTTGCAAGCT |
| | CCAGTAGCTCCGCGTGTCTCATTTGCCAGGAAGATGGGTTCCCACGTAGCAAATCGTACA |
| | TTGTGCCCTGTAGCTCCTTAGCTAGTTAGCTCACAAGCCGTGTTTTATGACTAATCCTTAA |
| | TAACTATGGTAAATAACTGTGACTGTGGGGTTTTTAATCTCTTGTCATTCTCATCCAAAAG |
| | TGACCAGCATACCAGTTCTTGCAATAAGATATTACCCTCAGAATATTAAGCACATTATTGT |
| | AGAGAAAAAAAAATATGTGTACACATATGAACGCACAACATGCACATTCATCCTCACATG |
| | TGGCACGTAAGGTCTCATTTGATATTGTGTAGGAAATCTGAAGCCTTTTCCTGAGGTCAT |
| | CTGTAAAATAGTCTCATTGCCAAGGCATCCCCAGTGCCAGCTGGTGAATCCATGATCAAA |
| | ATGCATACGTATTGTTAAATGATAAGGTTTAGAATGACAGGAACCCATCACTGTGTCTCA |
| | TGGTCCCACTTCCCCATCTGTGTGTGAATTCCTTTAGACTAAGGGCAGGAAGACTTCCAG |
| | CTTTCTCTTTGTTCTTCAATGTGAAACTGAGACCAAGTCTCTCTAAGACAAATGCAGTGTA |
| | TTTAATGTTTGTAAGCAATTCTAAGTGAGATGTTTGGCAAGAAATCCCCTAACTGATTTCC |
| | ATCCAAACCTACCTTATAGAGCACAATATTAAGTGTTGTACAATTACTGTGAGAACTGTG |
| | AATATGTGTAACTTTTTTTTAGTATTTGCCCGGGGGGAAAAAGATATTGTATTATCATATA |
| | TGCTTTTTTGCAATAAGGATTTATTCTCAGAACACCAAGTAAATCTATCTCTATATAAAAA |
| | ATATATGTAATATATACATATTCAAAGTATATACAGAGCCTGTTTTAAAAAATACAGTATT |
| | ATTTAGTAAAATTATCTGTTCTATGGACCAAATGTAAAATATTTATAAATGAAGATGCATT |
| | TTAAATGTCTATAAATGGTGTCATAACTAGAGCACGGGCGTTATGTAAGTTTCTAAGAAT |
| | TTAGAGGATAAATAATAAAGGTTCTATGATATACAA |
| SEQ ID NO: | TTTCAATATTATTGAAGCATTTATCAGGGTTATTGTCTCATGAGCGGATACATATTTGAAT |
| 30 | GTATTTAGAAAAATAAACAAATAGGGGTCAGTGTTACAACCAATTAACCAATTCTGAACA |
| AAV true | TTATCGCGAGCCCATTTATACCTGAATATGGCTCATAACACCCCTTGTTTGCCTGGCGGCA |
| type DNA | GTAGCGCGGTGGTCCCACCTGACCCCATGCCGAACTCAGAAGTGAAACGCCGTAGCGCC |
| sequence | GATGGTAGTGTGGGGACTCCCCATGCGAGAGTAGGGAACTGCCAGGCATCAAATAAAA |
| | CGAAAGGCTCAGTCGAAAGACTGGGCCTTTCGCCCGGGCTAATTAGGGGGTGTCGCCCT |
| | TCGCTGAAGTCCTGTATTAGAGGTCACGTGAGTGTTTTGCGACATTTTGCGACACCATGT |
| | GGTCACGCTGGGTATTTAAGCCCGAGTGAGCACGCAGGGTCTCCATTTTGAAGCGGGAG |
| | GTTTGAACGCGCAGCCGCCATGCCGGGGTTTTACGAGATTGTGATTAAGGTCCCCAGCG |
| | ACCTTGACGAGCATCTGCCCGGCATTTCTGACAGCTTTGTGAACTGGGTGGCCGAGAAG |
| | GAATGGGAGTTGCCGCCAGATTCTGACATGGATCTGAATCTGATTGAGCAGGCACCCCT |
| | GACCGTGGCCGAGAAGCTGCAGCGCGACTTTCTGACGGAATGGCGCCGTGTGAGTAAG |
| | GCCCCGGAGGCCCTTTTCTTTGTGCAATTTGAGAAGGGAGAGAGCTACTTCCACATGCAC |
| | GTGCTCGTGGAAACCACCGGGGTGAAATCCATGGTTTTGGGACGTTTCCTGAGTCAGAT |
| | TCGCGAAAAACTGATTCAGAGAATTTACCGCGGGATCGAGCCGACTTTGCCAAACTGGT |
| | TCGCGGTCACAAAGACCAGAAATGGCGCCGGAGGCGGGAACAAGGTGGTGGATGAGT |
| | GCTACATCCCCAATTACTTGCTCCCCAAAACCCAGCCTGAGCTCCAGTGGGCGTGGACTA |
| | ATATGGAACAGTATTTAAGCGCCTGTTTGAATCTCACGGAGCGTAAACGGTTGGTGGCG |
| | CAGCATCTGACGCACGTGTCGCAGACGCAGGAGCAGAACAAAGAGAATCAGAATCCCA |
| | ATTCTGATGCGCCGGTGATCAGATCAAAAACTTCAGCCAGGTACATGGAGCTGGTCGGG |
| | TGGCTCGTGGACAAGGGGATTACCTCGGAGAAGCAGTGGATCCAGGAGGACCAGGCCT |
| | CATACATCTCCTTCAATGCGGCCTCCAACTCGCGGTCCCAAATCAAGGCTGCCTTGGACA |
| | ATGCGGGAAAGATTATGAGCCTGACTAAAACCGCCCCCGACTACCTGGTGGGCCAGCAG |
| | CCCGTGGAGGACATTTCCAGCAATCGGATTTATAAAATTTTGGAACTAAACGGGTACGAT |
| | CCCCAATATGCGGCTTCCGTCTTTCTGGGATGGGCCACGAAAAAGTTCGGCAAGAGGAA |
| | CACCATCTGGCTGTTTGGGCCTGCAACTACCGGGAAGACCAACATCGCGGAGGCCATAG |
| | CCCACACTGTGCCCTTCTACGGGTGCGTAAACTGGACCAATGAGAACTTTCCCTTCAACG |
| | ACTGTGTCGACAAGATGGTGATCTGGTGGGAGGAGGGGAAGATGACCGCCAAGGTCGT |
| | GGAGTCGGCCAAAGCCATTCTCGGAGGAAGCAAGGTGCGCGTGGACCAGAAATGCAAG |
| | TCCTCGGCCCAGATAGACCCGACTCCCGTGATCGTCACCTCCAACACCAACATGTGCGCC |
| | GTGATTGACGGGAACTCAACGACCTTCGAACACCAGCAGCCGTTGCAAGACCGGATGTT |
| | CAAATTTGAACTCACCCGCCGTCTGGATCATGACTTTGGGAAGGTCACCAAGCAGGAAG |
| | TCAAAGACTTTTTCCGGTGGGCAAAGGATCACGTGGTTGAGGTGGAGCATGAATTCTAC |
| | GTCAAAAAGGGTGGAGCCAAGAAAAGACCCGCCCCCAGTGACGCAGATATAAGTGAGC |
| | CCAAACGGGTGCGCGAGTCAGTTGCGCAGCCATCGACGTCAGACGCGGAAGCTTCGATC |
| | AACTACGCAGACAGGTACCAAAACAAATGTTCTCGTCACGTGGGCATGAATCTGATGCT |
| | GTTTCCCTGCAGACAATGCGAGAGAATGAATCAGAATTCAAATATCTGCTTCACTCACGG |
| | ACAGAAAGACTGTTTAGAGTGCTTTCCCGTGTCAGAATCTCAACCCGTTTCTGTCGTCAA |
| | AAAGGCGTATCAGAAACTGTGCTACATTCATCATATCATGGGAAAGGTGCCAGACGCTT |
| | GCACTGCCTGCGATCTGGTCAATGTGGATTTGGATGACTGCATCTTTGAACAATAAATGA |
| | TTTAAATCAGGTATGGCTGCCGATGGTTATCTTCCAGATTGGCTCGAGGACACTCTCTCT |
| | GAAGGAATAAGACAGTGGTGGAAGCTCAAACCTGGCCCACCACCACCAAAGCCCGCAG |
| | AGCGGCATAAGGACGACAGCAGGGGTCTTGTGCTTCCTGGGTACAAGTACCTCGGACCC |
| | TTCAACGGACTCGACAAGGGAGAGCCGGTCAACGAGGCAGACGCCGCGGCCCTCGAGC |
| | ACGACAAAGCCTACGACCGGCAGCTCGACAGCGGAGACAACCCGTACCTCAAGTACAAC |
| | CACGCCGACGCGGAGTTTCAGGAGCGCCTTAAAGAAGATACGTCTTTTGGGGGCAACCT |
| | CGGACGAGCAGTCTTCCAGGCGAAAAAGAGGATCCTTGAACCTCTGGGCCTGGTTGAGG |
| | AACCTGTTAAGACGGCTCCGGGAAAAAAGAGGCCGGTAGAGCACTCTCCTGCCGAGCCA |
| | GACTCCTCCTCGGGAACCGGAAAGAGCGGCCAGCAGCCTGCAAGAAAAAGATTGAATTT |
| | TGGTCAGACTGGAGACGCAGACTCAGTACCTGACCCCCAGCCTCTCGGACAGCCACCAG |
| | CAGCCCCCTCTGGTCTGGGAACTAATACGATGGCTAGCGGCAGTGGCGCACCAATGGCA |
| | GACAATAACGAGGGCGCCGACGGAGTGGGTAATTCCTCGGGAAATTGGCATTGCGATTC |
| | CACATGGATGGGCGACAGAGTCATCACCACCAGCACCCGAACCTGGGCCCTGCCCACCT |
| | ACAACAACCACCTCTACAAACAAATTTCCAGCCAATCAGGAGCCTCGAACGACAATCACT |
| | ACTTTGGCTACAGCACCCCTTGGGGGTATTTTGACTTCAACAGATTCCACTGCCACTTTTC |
| | ACCACGTGACTGGCAAAGACTCATCAACAACAACTGGGGATTCCGACCCAAGAGACTCA |
| | GCTTCAAGCTCTTTAACATTCAAGTCAAAGAGGTCACGCAGAATGACGGTACGACGACG |
| | ATTGCCAATAACCTTACCAGCACGGTTCAGGTGTTTACTGACTCGGAGTACCAGCTCCCG |
| | TACGTCCTCGGCTCGGCGCATCAAGGATGCCTCCCGCCGTTCCCAGCAGACGTCTTCATG |
| | GTGCCACAGTATGGATACCTCACCCTGAACAACGGGAGTCAGGCAGTAGGACGCTCTTC |
| | ATTTTACTGCCTGGAGTACTTTCCTTCTCAGATGCTGCGTACCGGAAACAACTTTACCTTC |
| | AGCTACACTTTTGAGGACGTTCCTTTCCACAGCAGCTACGCTCACAGCCAGAGTCTGGAC |
| | CGTCTCATGAATCCTCTCATCGACCAGTACCTGTATTACTTGAGCAGAACAAACACTCCAA |
| | GTGGAACCACCACGATGTCAAGGCTTCAGTTTTCTCAGGCCGGAGCGAGTGACATTCGG |
| | GACCAGTCTAGGAACTGGCTTCCTGGACCCTGTTACCGCCAGCAGCGAGTATCAAAGAC |
| | AGCCGCGGATAACAACAACAGTGACTACTCGTGGACTGGAGCTACCAAGTACCACCTCA |
| | ATGGCAGAGACTCTCTGGTGAATCCGGGCCCGGCCATGGCAAGCCACAAGGACGATGA |
| | AGAAAAGTACTTTCCTCAGAGCGGGGTTCTCATCTTTGGGAAGCAAGACTCAGGCAAAA |
| | CAAATGTGGACATTGAAAAGGTCATGATTACAGACGAAGAGGAAATCAGGACAACCAAT |
| | CCCGTGGCTACGGAGCAGTATGGTTCTGTATCTACCAACCTCCAGAGCGGCAACACCCAA |
| | GCAGCTACCAGCGATGTCAACACACAAGGCGTTCTTCCAGGCATGGTCTGGCAGGACAG |
| | AGATGTGTACCTTCAGGGGCCCATCTGGGCAAAGATTCCACACACGGACGGACATTTTC |
| | ACCCCTCTCCCCTCATGGGTGGATTCGGACTTAAACACCCTCCTCCACAGATTCTCATCAA |
| | GAACACCCCGGTACCTGCGAATCCTTCGACCACCTTCAGTGCGGCAAAGTTTGCTTCCTTC |
| | ATCACACAGTACTCCACGGGACAGGTCAGCGTGGAGATCGAGTGGGAGCTGCAGAAGG |
| | AAAACAGCAAACGCTGGAATCCCGAAATTCAGTACACTTCCAACTACAACAAGTCTGTTA |
| | ATGTGGACTTTACTGTGGACACTAATGGCGTGTATTCAGAGCCTCGCCCCATTGGCACCA |
| | GATACCTGACTCGTAATCTGTAATTGCTTGTTAATCAATAAACCGTTTAATTCGTTTCAGTT |
| | GAACTTTGGTCTCTGCGCGTCAAAAGGGCGACACAAAATTTATTCTAAATGCATAATAAA |
| | TACTGATAACATCTTATAGTTTGTATTATATTTTGTATTATCGTTGACATGTATAATTTTTCT |
| | AGAGCGGCCGCAGATCTCAGCTGGATATCAAAAACTGATTTTCCCTTTATTATTTTCGAG |
| | ATTTATTTTCTTAATTCTCTTTAACAAACTAGAAATATTGTATATACAAAAAATCATAAATA |
| | GTGCGTTGCTTTTTTCTCATTTATAAGGTTAAATAATTCTCATATATCAAGCAAAGTGACA |
| | GGCGCCCTTAAATATTCTGACAAATGCTCTTTCCCTAAACTCCCCCCATAAAAAAACCCGC |
| | CGAAGCGGGTTTTTACGTTATTTGCGGATTAACGATTACTCGTTATCAGAACCGCCCAGG |
| | GGGCCCGAGCTTAAGACTGGCCGTCGTTTTACAACACAGAAAGAGTTTGTAGAAACGCA |
| | AAAAGGCCATCCGTCAGGGGCCTTCTGCTTAGTTTGATGCCTGGCAGTTCCCTACTCTCG |
| | CCTTCCGCTTCCTCGCTCACTGACTCGCTGCGCTCGGTCGTTCGGCTGCGGCGAGCGGTA |
| | TCAGCTCACTCAAAGGCGGTAATACGGTTATCCACAGAATCAGGGGATAACGCAGGAAA |
| | GAACATGTGAGCAAAAGGCCAGCAAAAGGCCAGGAACCGTAAAAAGGCCGCGTTGCTG |
| | GCGTTTTTCCATAGGCTCCGCCCCCCTGACGAGCATCACAAAAATCGACGCTCAAGTCAG |
| | AGGTGGCGAAACCCGACAGGACTATAAAGATACCAGGCGTTTCCCCCTGGAAGCTCCCT |
| | CGTGCGCTCTCCTGTTCCGACCCTGCCGCTTACCGGATACCTGTCCGCCTTTCTCCCTTCG |
| | GGAAGCGTGGCGCTTTCTCATAGCTCACGCTGTAGGTATCTCAGTTCGGTGTAGGTCGTT |
| | CGCTCCAAGCTGGGCTGTGTGCACGAACCCCCCGTTCAGCCCGACCGCTGCGCCTTATCC |
| | GGTAACTATCGTCTTGAGTCCAACCCGGTAAGACACGACTTATCGCCACTGGCAGCAGCC |
| | ACTGGTAACAGGATTAGCAGAGCGAGGTATGTAGGCGGTGCTACAGAGTTCTTGAAGT |
| | GGTGGGCTAACTACGGCTACACTAGAAGAACAGTATTTGGTATCTGCGCTCTGCTGAAG |
| | CCAGTTACCTTCGGAAAAAGAGTTGGTAGCTCTTGATCCGGCAAACAAACCACCGCTGGT |
| | AGCGGTGGTTTTTTTGTTTGCAAGCAGCAGATTACGCGCAGAAAAAAAGGATCTCAAGA |
| | AGATCCTTTGATCTTTTCTACGGGGTCTGACGCTCAGTGGAACGACGCGCGCGTAACTCA |
| | CGTTAAGGGATTTTGGTCATGAGCTTGCGCCGTCCCGTCAAGTCAGCGTAATGCTCTGCT |
| | TTTAGAAAAACTCATCGAGCATCAAATGAAACTGCAATTTATTCATATCAGGATTATCAAT |
| | ACCATATTTTTGAAAAAGCCGTTTCTGTAATGAAGGAGAAAACTCACCGAGGCAGTTCCA |
| | TAGGATGGCAAGATCCTGGTATCGGTCTGCGATTCCGACTCGTCCAACATCAATACAACC |
| | TATTAATTTCCCCTCGTCAAAAATAAGGTTATCAAGTGAGAAATCACCATGAGTGACGAC |
| | TGAATCCGGTGAGAATGGCAAAAGTTTATGCATTTCTTTCCAGACTTGTTCAACAGGCCA |
| | GCCATTACGCTCGTCATCAAAATCACTCGCATCAACCAAACCGTTATTCATTCGTGATTGC |
| | GCCTGAGCGAGGCGAAATACGCGATCGCTGTTAAAAGGACAATTACAAACAGGAATCG |
| | AGTGCAACCGGCGCAGGAACACTGCCAGCGCATCAACAATATTTTCACCTGAATCAGGA |
| | TATTCTTCTAATACCTGGAACGCTGTTTTTCCGGGGATCGCAGTGGTGAGTAACCATGCA |
| | TCATCAGGAGTACGGATAAAATGCTTGATGGTCGGAAGTGGCATAAATTCCGTCAGCCA |
| | GTTTAGTCTGACCATCTCATCTGTAACATCATTGGCAACGCTACCTTTGCCATGTTTCAGA |
| | AACAACTCTGGCGCATCGGGCTTCCCATACAAGCGATAGATTGTCGCACCTGATTGCCCG |
| | ACATTATCGCGAGCCCATTTATACCCATATAAATCAGCATCCATGTTGGAATTTAATCGCG |
| | GCCTCGACGTTTCCCGTTGAATATGGCTCATATTCTTCCTT |
| SEQ ID NO: | ATGGCGACTGACAACAGCAAGGTGGCTGATGGGCAGATCTCTACTGAGGTCAGCGAGG |
| 31 | CCCCTGTGGCCAGCGACAAGCCCAAAACCCTGGTAGTCAAGGTGCAGAAGAAGGCCGG |
| Mouse | GGACCTCCCTGACCGGGACACATGGAAGGGACGCTTCGACTTCCTCATGTCCTGCGTGG |
| SLC6A1 NCBI | GCTATGCCATCGGCCTGGGCAATGTGTGGAGGTTCCCTTACCTCTGTGGGAAAAACGGT |
| Reference | GGCGGGGCCTTCCTAATCCCATATTTCCTGACGCTCATCTTTGCGGGTGTTCCTCTCTTCC |
| NM_178703.4 | TTTTGGAGTGCTCCCTAGGCCAGTACACCTCCATTGGGGGCCTGGGCGTATGGAAGCTG |
| | GCGCCCATGTTCAAGGGTGTGGGCCTCGCGGCAGCTGTGCTGTCCTTCTGGCTGAACATC |
| | TACTACATCGTCATCATCTCCTGGGCCATCTACTACCTGTACAACTCCTTCACCACGACCCT |
| | GCCATGGAAACAGTGTGACAACCCGTGGAACACTGACCGCTGCTTCTCCAACTACAGCCT |
| | GGTCAATACCACCAACATGACCAGCGCCGTGGTGGAGTTCTGGGAGCGCAACATGCACC |
| | AGATGACAGATGGACTGGACAAGCCAGGACAGATCCGCTGGCCTCTGGCCATCACACTG |
| | GCCATTGCCTGGGTGCTCGTGTATTTCTGCATCTGGAAGGGTGTTGGTTGGACTGGAAA |
| | GGTGGTCTACTTCTCAGCCACGTACCCCTACATCATGCTTATCATCCTGTTCTTCCGTGGA |
| | GTGACGCTTCCCGGGGCCAAGGAGGGGATCCTCTTCTACATCACACCCAACTTCCGAAA |
| | ATAGATGAATAGTTTAATTATAGGTGTTCATCAATCGAAAAAGCAACGTATCTTATTTAAA |
| | GCTGTCTGATTCTGAGGTGTGGCTTGACGCCGCCACCCAGATCTTCTTCTCCTACGGGCT |
| | GGGCCTGGGGTCCCTGATTGCTCTGGGAAGCTACAACTCTTTCCACAACAATGTGTACAG |
| | GGACTCCATCATCGTTTGCTGCATCAACTCCTGCACCAGCATGTTTGCCGGATTCGTCATC |
| | TTCTCCATCGTGGGCTTCATGGCTCATGTCACCAAGAGGTCCATAGCTGATGTGGCAGCC |
| | TCAGGCCCGGGGCTGGCATTCTTGGCGTACCCTGAGGCTGTGACACAGCTACCCATCTCT |
| | CCCCTCTGGGCTATCCTCTTCTTCTCCATGCTGCTGATGCTGGGCATTGACAGCCAGTTCT |
| | GTACCGTGGAGGGCTTCATCACTGCCCTGGTGGACGAGTACCCCAGACTTCTCCGCAATC |
| | GCCGTGAACTCTTCATTGCTGCCGTGTGCATCGTGTCCTACCTGATTGGCCTGTCTAACAT |
| | CACCCAGGGTGGCATTTATGTCTTCAAACTGTTTGATTATTACTCTGCCAGCGGCATGAG |
| | CTTGCTGTTCCTGGTTTTCTTCGAGTGTGTCTCCATTTCCTGGTTTTATGGTGTCAACCGGT |
| | TCTATGACAACATCCAGGAGATGGTTGGCTCCAGGCCCTGCATCTGGTGGAAGCTGTGC |
| | TGGTCCTTTTTCACACCCATCATTGTGGCGGGCGTGTTTCTCTTCAGTGCTGTGCAGATGA |
| | CACCACTCACCATGGGAAGCTATGTTTTCCCCAAGTGGGGCCAGGGCGTGGGCTGGCTC |
| | ATGGCTCTGTCCTCCATGGTGCTCATCCCCGGGTACATGGCTTACATGTTCCTCACCCTGA |
| | AGGGCTCCCTGAAGCAGCGTCTCCAGGTCATGATTCAGCCCAGTGAAGATATTGTGCGC |
| | CCTGAGAATGGCCCTGAGCAGCCGCAGGCTGGCAGCTCAGCCAGCAAGGAGGCCTACA |
| | TCTAG |
| SEQ ID NO: | GAGCAGAAACTCATCTCAGAAGAGGATCTG |
| 32 | |
| Myc tag | |
| SEQ ID NO: | TACCCTTACGATGTACCGGATTACGCA |
| 33 | |
| HA tag | |
| SEQ ID NO: | GCGCTCCCTCCTCTCGGAGAGAGGGCTGTGGTAAAACCCGTCCGGAAATTGGCCGCCGC |
| 34 | TGCCGCCACCGCCGCCGCCGCCGCCGCGCCGAGCGGAGGAGGAGGAGGAGGCGAGGA |
| MECP2 | GGAGAGACTGTGAGTGGGACCGCCAAGGCCGCGGGCGGGGACCCTTGCTGGGGGGCG |
| intron | GGTAGGGGCGGGACGTGGCGCGGGAGGGGCCCGCGGGGTCGGGCGACACGGCTGGC |
| | GGTTGGCGTCCCTCCTCTCTACCCTCCCCCTCCCTCTGCCGCCGGTGGTGGCTTTCTCCAC |
| | TCGTCTCCCGCAATCGCGAGCGACGGTTCTCAGCGCGATCTCCCTGGAGCCACCTTCGAT |
| | TGACGCCCTCCCGCTGCCCGCCCCATCTGTGCGCATCCTAGGCCCCAGCTGTGCAAGCGC |
| | CCTTGTCGTCTGGGCTTCGCCAGTTGGGGCTGCGCGCGCTCCTGCCCTTCTTGGGGCTTT |
| | GGGCCTCGGCACTGTCGCGCGCCCGCGGTCCCGGCCTCTCCCTGGATCGCGCTGTCCCCT |
| | TCTCCCTCGCGCGCCCCCACTCCCGTTACTTGCTCCCCCCTCACACACACAGACTGGCGCG |
| | CGTGCGCAGTCCATCTCCCGTTGGGAGAGTGCGCCACAAGGGCTCCTGAGCTCTTACCCC |
| | CATCTCTGGGTTTTGCTCCCTCCTCCTCCTCTCCCATTCCGTGACTTTTTGCCCCCACTGCA |
| | AGCGAGTCGGTCCATCAGCTCCATTCCCCACTTGGCAGGAACAAGTTGAGGGTTATTGTC |
| | CACCCACAAAAAGGACTAGACATTTTGTTCCTAGGTCCCACAACTCATCATAAAGAGTTG |
| | GTTGTAGTTCTCATCAGGAACCGTGGGCAAGGGACTGTGCGTTCCTCAGCACTCGAAGC |
| | TCTTCCGTGAGACCTTGCCCGCAGGGTGCTCTGGTTCTTTGGGGTTGCTGTGCTGTGGCT |
| | TCGGAATTTGAGCGTCTTCCCACCCTCCCTCCCCTCCCTTCGCCAGCGTTCTGTCTACAAG |
| | AAAGAATAGGCAGGTGTCCTTGGATATCGTAGTTGCTAATCGCCTATACACTGTTCTATT |
| | ACACCTTTCTGCTAAGGATAGGGTTTTTGGTTTTGGTTTTGGTTTTGTTCCCCACCCTCCA |
| | GTTTGGTTTAGTTTTGGTTTTGGCATTTAGGGTTTTTTGGGGGGGAGTAATATCTTGTGG |
| | TAAAGACCCATCTGACCCAAGATACCTTTTTTCTCATACTGGAACCCTAGGCAGCAGTTGC |
| | TATTTCCCTGAGTTAGCAATAGTTTTACAGTATTTTGAGGCCTTTTGTCCATAATTCTCACG |
| | GAATCCCTCAGGGATCAGATTAGCTGCTGTTGGGATCAGGAAATTGGGTTACACCGCTG |
| | AAATCTCTTGCTGGGGCCCTTGTTTTGAATTGGAAAGTCAGGAGGCTGGAACGAAGGCT |
| | CACAAGTTAACAGTGCCAGCTGCTCTTCCAGAAGCCCTGGATTCAGTCCCACCAATCCAT |
| | CGCGGGTCACAACCATCTGTAACTTCAGTCCCAAGGGGTCCGAAGCCCTCTTCTGGCTTT |
| | GCCCTATTATTTTATTTATCTTATCTGTTTTTGTCTTGTCATCTGGCAAGCCCAGGGGGCCA |
| | TTGGGTGCAACTTATAAACTGACTTCTGTATCTTAAGAAGCCAACCATACAGTGCTTACAT |
| | TCCAGAAAAAAAATCTGCCACTTTAACAGCACTAGAACTAGGGTTTAGAGAAGTATCATA |
| | AAGGTCAAATATCTTTGACCAATATCACCAGCAACCTAAAGCTGTTAAGAAATCTTTGGG |
| | CCCCAGCTTGACCCAAGGATACAGTATCCTAGGGAAGTTACCAAAATCAGAGATAGTAT |
| | GCAGCAGCCAGGGGTCTCATGTGTGGCACTCAAGCTCACCTATACTCACTACTGTGCAGA |
| | CAGCTGTGTTCTCTGTAATACTTACATATTTGTTTAATACTTCAGGGAGGAAAAGTCAGA |
| | AGACCAGGATCTCCAGGGCCTCA |
| SEQ ID NO: | GGCTCCGGTGCCCGTCAGTGGGCAGAGCGCACATCGCCCACAGTCCCCGAGAAGTTGG |
| 35 EF1a | GGGGAGGGGTCGGCAATTGAACCGGTGCCTAGAGAAGGTGGCGCGGGGTAAACTGGG |
| promoter | AAAGTGATGTCGTGTACTGGCTCCGCCTTTTTCCCGAGGGTGGGGGAGAACCGTATATA |
| | AGTGCACTAGTCGCCGTGAACGTTCTTTTTCGCAACGGGTTTGCCGCCAGAACACAG |

The invention will now be further described by way of examples with references to embodiments illustrated in the accompanying drawings.

### EXAMPLES

### Example 1: Construct Design, Generation and Cloning

Plasmids used in this study were constructed by recombinant DNA techniques. AAV Cis backbone plasmids were synthesized de-novo and contained two AAV inverted terminal repeats (ITRs), a kanamycin resistance cassette, a prokaryotic origin of replication, and an SV40 polyadenylation sequence. Human and mouse SLC6A1 DNA sequences (comprising SEQ ID NO: 15 and 31 (or 16), respectively), coding isoform a of GAT-1, were synthesized de-novo with convenient cloning restriction sites). Individual promoters were synthesized de-novo with convenient restriction sites. The Human influenza hemagglutinin (HA) or Myc tags (encoded according to SEQ ID NO: 33 and 32, respectively) were synthesized as oligonucleotides from Integrated DNA Technologies™ (Coralville, IA, USA) and inserted at the amino or carboxy terminal as indicated in FIG. 3. Four different promoters were tested for both human and mouse SLC6A1 gene.

### Example 2: Evaluation of SLC6A1 Expression Under Different Promoters

### Cell Culture

The human-derived AD-HEK293 (Agilent Technologies™, Santa Clara, CA, USA) and mouse-derived Neuro-2A (ATCC™, Manassas, VA) cell lines were passaged in DMEM+10% FBS+1% Penicillin/Streptomycin (all from Thermo Fisher Scientific™, Waltham, MA, USA). Neuro-2A cells were differentiated by supplementing the growth media with 10 μM Retinoic Acid (MilliporeSigma™, Burlington, MA, USA) for 72 hours as previously described (Tremblay, R. G. et al. Differentiation of mouse Neuro 2A cells into dopamine neurons. J Neurosci Methods 186, 60-67, doi:10.1016/j.jneumeth.2009.11.004 (2010)). Cells were transfected using Lipofectamine 2000 (Thermo Fisher Scientific™, Waltham, MA, USA) according to the manufacturer's protocol. A control transfection, without plasmid was also included.

### Immunofluorescence and Microscopy

Imaging experiments were performed on a Zeiss Axio Observer 7 epifluorescent microscope (Carl Zeiss AG™, Oberkochen, Germany) equipped with a 20× objective lens, and a Hamamatsu Orca 4 flash cooled monochrome camera (Hamamatsu Photonics KK™, Hamamatsu City, Japan). Transfected AD-HEK293 and Neuro-2A cells were fixed with 4% paraformaldehyde (Electron Microscopy Sciences, Hatfield, PA, 19440), and stained with the primary antibodies rabbit monoclonal anti-GAT-1 (Abcam™, Cambridge, MA, USA) at 1:100 or rabbit polyclonal anti-GAT-1 (Cell Signalling Technology™, Danvers, MA, USA) at 1:100. Cells were then stained with goat anti-rabbit secondary antibodies conjugated to Alexa Fluor 488 or 568 at 1:1,000 prior to imaging.

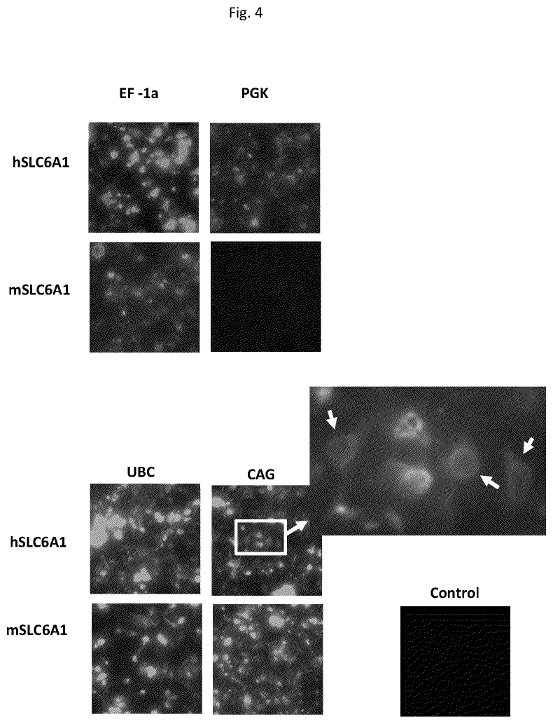

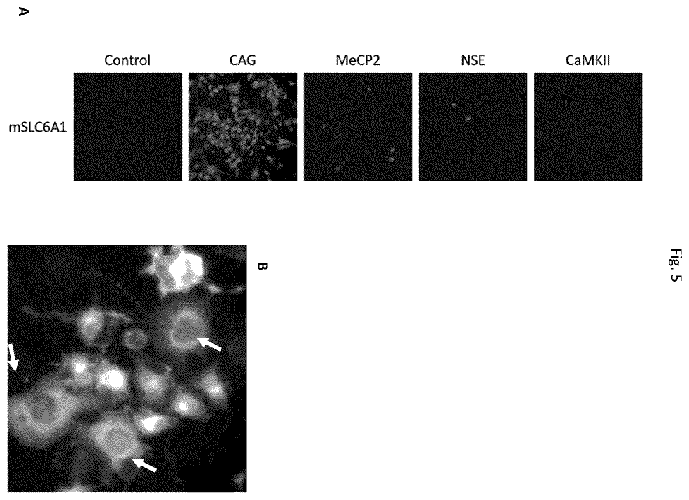

As shown in FIG. 4, all transfected cells demonstrated robust expression of the mouse and human transgene under the control of the ubiquitous EF1a, PGK, UBC and CAG promoters. The strongest expression was observed with the CAG promoter, with lower expression observed, as expected, with the PGK promoter.

Neuro-2A transfected cells transfected with the mSLC6A1 plasmids driven by different neuron-specific promoters and CAG ubiquitous promoter were also analysed. As shown in FIG. 5, all promoters lead to the expression of mouse SLC6A1; as expected, the neuron-specific promotors were weaker compared to the strong and ubiquitous CAG promoter. Enlarged images of transfected AD-HEK293 and Neuro-2A show that SLC6A1 expressed from these plasmids localizes to the plasma membrane as expected (FIGS. 4 and 5B).

### Western-Blot Analysis

Transfected AD-HEK 293 cells were harvested in 1× Cell Lysis Buffer (Cell Signaling Technology™, Danvers, MA, USA) containing 1× Halt Protease and Phosphatase Inhibitor Cocktail (Thermo Fisher Scientific™, Waltham, MA, USA) according to the manufacturer's instructions. Lithium dodecyl sulfate (LDS) Sample Buffer supplemented with 10% reducing agent (both Thermo Fisher Scientific™, Waltham, MA, US) were added to the protein lysates to a final concentration of 1×. Samples were resolved by 1D SDS-PAGE gel electrophoresis. For each sample, 30 μg of proteins were loaded per lane. Proteins were transferred to nitrocellulose membranes (Li-Cor Biosciences™, Lincoln, NE, USA) using a semidry transfer apparatus (Bio-Rad Laboratories™, Hercules CA). Following transfer, membranes were incubated in blocking solution (Li-Cor Biosciences™, Lincoln, NE, USA) for 1 hour at room temperature. Membranes were then incubated with blocking solution containing primary antibodies overnight at 4° C. The following primary antibodies were used for this analysis: rabbit monoclonal anti-GAT-1 (Abcam™, Cambridge, MA, USA) at 1:1,000, rabbit polyclonal anti-GAT-1 (Cell Signalling Technology™, Danvers, MA, USA) at 1:1,000, rabbit polyclonal anti-c-myc at 1,1000 (MilliporeSigma™, Burlington, MA, USA), rabbit monoclonal anti-HA at 1:1,000 (Cell Signalling Technology™, Danvers, MA, USA), mouse monoclonal anti-GAPDH at 1:1,000 (Thermo Fisher Scientific™, Waltham, MA, US), rabbit monoclonal anti-GAPDH at 1:1,000 (Cell Signalling Technology™, Danvers, MA, USA). Membranes were washed three times with PBST solution, placed in blocking solution containing IRDye 800CW or 680LT goat anti-mouse or goat anti-chicken secondary antibodies (1:15,000; Li-Cor Biosciences™, Lincoln, NE, USA) suitable for detection on the far-red spectrum for 1 hour at room temperature. Proteins were visualized using a Li-Cor Odyssey CLx far red imager (Li-Cor Biosciences™, Lincoln, NE, USA.

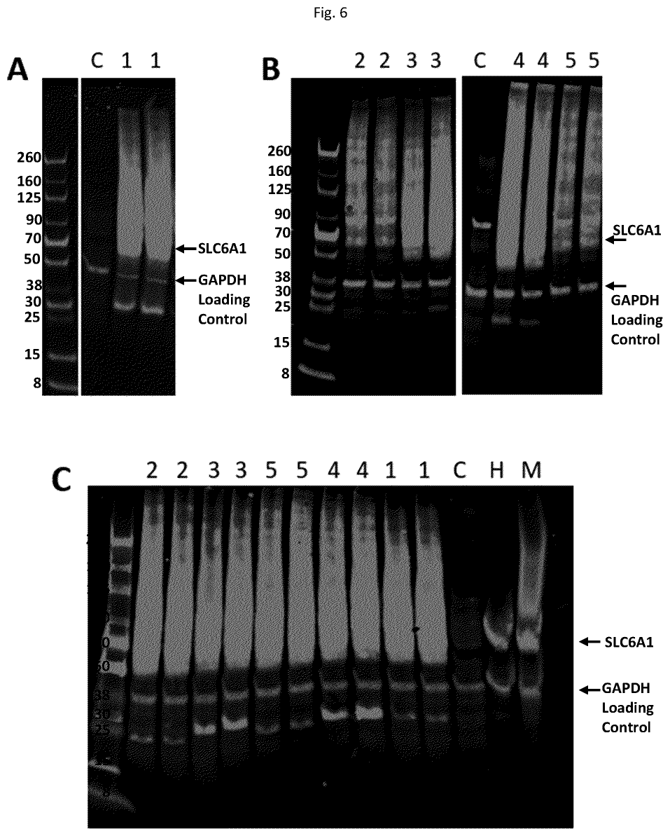

SLC6A1 is a membrane protein with 12 transmembrane domains and is glycosylated (Bennett, E. R. and B. I. Kanner. J Biol Chem. 272, 1203-1210, (1997)). The molecular mass of the SLC6A1 monomer under reducing conditions is predicted at ˜70 kDa and the protein was detected by Western blot as a dimer and high molecular mass aggregates, presumably due to its membrane topology and post-translational modifications. This is consistent with the banding pattern that was detected for SLC6A1 in the literature (Bennett, E. R. and B. I. Kanner. J Biol Chem. 272, 1203-1210, (1997). Additional bands detected in some of the conditions at a lower molecular weight around ˜28 kDA are likely degradation products of SLC6A1. Detection of GAPDH was used as a loading control. These results show that robust expression was achieved by both the N- and C-terminal tagged constructs driven by the CAG promoter (FIG. 6). Similar results were obtained when the protein was detected using antibodies against SLC6A1 in brain lysates from human and mouse samples (panel C lanes labelled H and M).

### Example 3: Pathogenic, Likely Pathogenic and Naturally Occurring Variant Identification and Analysis

The ClinVar database (https://www.ncbi.nlm.nih.gov/clinvar/), a freely accessible, public archive of reports of the relationships among human variations and phenotypes, with supporting evidence, was mined to identify SLC6A1 gene variants using search term “SLC6A1” and “pathogenic” or “likely pathogenic”. The list of pathogenic variants was complemented with mutations published in scientific peer-reviewed literature and manually curated from a PubMed (https://pubmed.ncbi.nlm.nih.gov/) search using search terms “SLC6A1 and mutation” and defined as pathogenic by the authors to identify additional SLC6A1 pathogenic variants not reported in ClinVar.

The pathogenic and likely pathogenic variants resulted from an amino acid change in the GAT-1 protein were then identified (Table 2A). Other pathogenic variants generated by a frame shift, or a deletion of an amino acid, or a mutation leading to the generation of a stop codon are shown in Table 2B.

TABLE 2A
Pathogenic and likely pathogenic variants resulting from
amino acid change (with reference to SEQ ID NO: 18)

| | |
|---|---|
| R44W | G297R |
| R50L | A305T |
| S56F | G307R |
| G63S | V323I |
| N66D | A334P |
| G75R | V342M |
| G79R | A357V |
| F92S | G362R |
| G94E | L366V |
| G105S | F385L |
| Q106R | G393S |
| Y140C | S456R |
| C173Y | S459R |
| F270S | M487T |
| R277H | V511L |
| A288V | G550R |
| S295L | G79V |
| D52E | A367T |
| D52V | G112V |
| F53S | G232V |

TABLE 2B
Pathogenic and likely pathogenic variants
resulted from other mutations

| | |
|---|---|
| A358fs | N66fs |
| A9fs | R419fs |
| G457fs | R50fs |
| L113fs | S175fs |
| L408fs | V446fs |
| C74* | Q534* |
| Q397* | R479* |
| W135* | W235* |
| W500* | W532* |
| Y246* | Q199* |
| Q291* | I268fs |
| I148fs | S437fs |
| L363fs | T170fs |
| F174del | F294del |

In addition to mutation due from amino acid changes, other mutations may occur. One type of mutation that was identified was a mutation involving the insertion or deletion of a nucleotide in which the number of changed base pairs is not divisible by three which lead to the creation of a new amino acid (a frameshift, indicated as fs in Table 2B). If the mutation disrupts the correct reading frame, the entire DNA sequence following the mutation will be read incorrectly. More specifically, A358fs as indicated in Table 2B, means that Alanine at position 358 with reference to SEQ ID NO: 18, is changed due to a frameshift of nucleotides, resulting in abnormal protein product with an incorrect amino acid sequence.

Another type of mutation identified was a mutation at the DNA level which removes one or more amino acid residues in the protein. This type of mutations is indicated as deletion (del) in Table 2B. For example, F174del means that phenylalanine at position 174 with reference to SEQ ID NO: 18 is removed, and the protein will be 1 amino acid shorter and missing Phe174.

Finally, another type of mutations identified was the introduction of a stop codon (indicated by an asterisk (*) in Table 2B), which is reported herein as for example C74* which means that a translation of the protein is stopped at Cysteine at position 74 with reference to SEQ ID NO: 18 and the protein will be truncated from this position onwards.

Naturally occurring variants in healthy population were derived from gnomAD (The Genome Aggregation Database—https://gnomad.broadinstitute.org/v2.1.1), a publicly available control data-set containing genetic information from 60,146 samples from unrelated individuals using the query term “SLC6A1”. The variants extracted from the control dataset include missense, resulting in amino acid change, start lost variants (a point mutation in the DNA sequence which results in the loss of AUG start codon, resulting in the reduction or elimination of GAT-1) and stop gained variants (a point mutation in the DNA sequence which results in a new stop codon, ultimately resulting in the reduction of GAT-1). The naturally occurring variants resulting in amino acid change are reported in Table 3.

TABLE 3
Naturally occurring variants (with reference to SEQ ID NO: 18)

| | | | | |
|---|---|---|---|---|
| Ala2Thr | Asp165Tyr | Arg277Ser | Ile434Met | Arg579His |
| Gly5Ser | Arg172Cys | Arg277Cys | Ser470Cys | Pro580Ser |
| Asp10Asn | Arg172His | Arg277Pro | Ile471Val | Pro587Ala |
| Gly11Arg | Phe174Tyr | Ser280Cys | Gly476Ser | Ala589Val |
| Ile13Thr | Ser178Asn | Asn310Ser | Arg479Gln | Ile599Val |
| Glu16Lys | Asn181Asp | Tyr317His | Lys497Asn | Met1 (start loss |
| | | | | variant) |
| Glu19Gly | Asn181Lys | Ile321Val | Phe502Tyr | Glu411 (stop |
| | | | | gained variant) |
| Pro21Thr | Arg195His | Ser328Leu | Ile506Val | |
| Lys33Glu | Met197Leu | Met332Val | Ala509Val | |
| Val34Leu | Asp202Glu | Val337Ile | Thr520Met | |
| Asp40Asn | Lys206Glu | His347Arg | Gly535Val | |
| Asp43Glu | Arg211Cys | Ala354Val | Leu547Phe | |
| Lys76Asn | Ile220Val | Leu375Met | Met552Ile | |
| Asn77Asp | Ile220Asn | Ile377Val | Met555Val | |
| Ile84Phe | Ala221Thr | Ile405Val | Thr558Asn | |
| Phe87Leu | Val240Ala | Val409Met | Arg566His | |
| Ile91Val | Phe242Val | Leu415Ile | Gln572Arg | |
| Val142Ile | Tyr246Cys | Arg417Cys | Pro573Thr | |
| Thr156Asn | Arg257Cys | Arg417His | Pro573Ser | |
| Thr158Pro | Arg257His | Arg419Cys | Ser574Asn | |
| Asp165Asn | Thr260Met | Arg419His | Val578Ile | |

### Example 4: Viral Particles Production

### AAV Production

Trans plasmids containing the AAV2 Rep sequences followed by the AAV9.hu14 (hereinafter AAV9) or AAV-true type (hereinafter AAVtt) capsid sequences (which amino acid sequence are SEQ ID NO: 24 and 25, respectively) were synthesized de-novo by ATUM™ (Newark, CA, USA). AAV helper plasmid pALD-X80 was purchased from Aldevron, LLC™ (Fargo, ND, USA).

Non-replicating AAV vectors were produced by the triple transfection method. Expi293 cells (Thermo Fisher™, Waltham, MA, USA) were passaged every 3-4 days using Expi293 Expression Media (Thermo Fisher™, Waltham, MA, USA) in shake flasks at a seeding density of 3.0E+05-3.5E+05 cells/mL. In the final passage prior to the start of the experiment, cells were passaged at 3.5E+05 cells/mL in 2×1,000 mL shake flasks at a total working volume of 220 mL per viral preparation. Viable cell density was calculated using a Vi-Cell Blu (Beckman Coulter™, Pasadena, CA, USA). One day prior to transfection, shake flasks were seeded at 1.5E+06 cells/mL.

A transfection complex was created for each flask as follows: 180 μL Polyethylenimine (PEI) MAX at 1 mg/mL (Polysciences Inc™, Warrington, PA, USA) was diluted in 1.5 mL OptiPRO serum free media (Thermo Fisher™, Waltham, MA, USA), vortexed at setting 8 four times and incubated for 5 minutes at room temperature. Separately, the 20 μg Cis plasmid (CAG-hSLC6A1), 30 μg Rep/Cap plasmid (AAV9 or AAV-tt), and 40 μg helper plasmid (pALD-X80) were diluted in 1.5 mL OptiPRO serum free media, vortexed at setting 8 four times and incubated for 5 minutes at room temperature. These two mixtures were then combined, vortexed at setting 8 four times, and incubated at room temperature for 15 minutes. Transfection complexes were then added to shake flasks containing cells. Cells were cultured with the transfection mixture at 37° C. with constant agitation at 125 rpm.

After 96 hours, flasks were spiked with AAV lysis buffer to a final concentration of 1× (150 mM NaCl, 120 mM Tris-HCl [pH=8.0], 2 mM MgCl2, 0.1% Triton X-100), and Benzonase (MilliporeSigma™, Burlington, MA, USA) to a final concentration of 50 U/mL. This mixture was incubated for 1 hour at 37° C. with constant agitation at 125 rpm. The mixture was clarified by centrifugation at 2,250×g for 20 minutes at 23° C. Samples were stored at −80° C. until further analysis.

### AAV Titer Determination

Each sample was removed from −80° C. and allowed to thaw at room temperature for 15 minutes. Once the sample was thawed, it was briefly vortexed and centrifuged for one minute. After this, 10 μL of sample was added to an individual well of a 96-well PCR plate combined with 10×DNase Buffer, 50 U DNase, and DNase-free water (all from Promega™, Madison, WI, USA) to a total volume of 100 μL in each well.

The plate was then transferred to a Bio-Rad™ (Hercules, CA, USA) thermal cycler and was heated for 30 minutes at 37° C. then cooled to 4° C. Samples were then serially diluted as described in the Table 4.

TABLE 4
Sample Dilution Scheme

| | Intermediate | | Intermediate | Diluent | Total | |
|---|---|---|---|---|---|---|
| Dilution | Dilution | Intermediate | Volume | Volume | Volume | Total |
| Step | Factor | Sample | (μL) | (μL) | (μL) | DF |
| D0 | 10 | DNase | 100 | NA | 100 | 10 |
| | | Treated | | | | |
| | | Sample | | | | |
| D1 | 1.5 | D0 | 100 | 50 | 150 | 1.50E+01 |
| D2 | 10 | D1 | 10 | 90 | 100 | 1.50E+02 |
| D3 | 3 | D2 | 10 | 90 | 100 | 1.50E+03 |
| D4 | 3 | D3 | 10 | 90 | 100 | 1.50E+04 |
| D5 | 3 | D4 | 10 | 90 | 100 | 1.50E+05 |

Five (5) μL of dilutions D2, D3, D4, and D5 were mixed with 20 μL of a ddPCR master mix composed of Supermix for Probes (No dUTP; Bio-Rad™, Hercules, CA, USA), forward primer GATCCAGACATGATAAGATACATTG, reverse primer GCAATAGCATCACAAATTTCAC, Probe 6-Fam/Zen/3′IB FQ: TGGACAAACCACAACTAGAATGCA, and DNase-free water to a final concentration of 1×. Each sample was run in duplicate in a 96-well PCR plate.

The plate was heat sealed with a foil covering, pulse vortexed, and centrifuged at 1,000×g for 5 minutes. The plate was placed into the Bio-Rad™ QX-200 droplet generator and droplets were generated per the manufacturer's instructions.

After droplet generation, the plate was heat-sealed with a foil covering and placed into a Bio-Rad™ thermocycler programmed to run the cycle described in Table 5.

TABLE 5
PCR amplification Settings (all ramping is set at 2.5° C./Seconds)

| Cycle Step | Temperature | | Duration | | Number of Cycles |
|---|---|---|---|---|---|
| Enzyme Activation | 95° | C. | 10 | minutes | 1 |
| Denaturing | 95° | C. | 30 | seconds | 39 |
| Annealing/Extension | 56° | C. | 1 | minute | |
| Enzyme Deactivation | 98° | C. | 10 | minutes | 1 |

| | | | | |
|---|---|---|---|---|
| Hold | 4° | C. | Infinite | 1 |

Once complete, the plate was placed into a Bio-Rad™ QX200 droplet for droplet reading per the manufacturer's instruction. The concentration of vector genomes (VG/mL) were quantified using the following formula:

VG/ML: X=[(aY)(1000/b)]D

where:
X is VG/mL;
a is volume of the ddPCR reaction (25 μl);
Y is the ddPCR readout in copies per microliter;
b is the volume of diluter vector in the ddPCR (5 μL);
D is the total dilution applied to the test material.

Assay acceptance criteria were defined as follows:

The % CV between the replicates must be ≤15%; if >15% one outlier may be omitted. If an outlier was omitted and the % CV remained >15%, the assay needed to be repeated. The inter-dilution % CV needed to be ≤20% and reported dilutions needed to be at least two consecutive dilutions. If the % CV was >20%, a dilution could be omitted so long the reported dilutions were at least two consecutive dilutions. If the averaged dilutions were still >20%, the assay needed to be repeated. Each reaction well needed to have ≥1,000 accepted droplets. If <10,000 droplets, the well was excluded from analysis.

### Viral Particle Quantitation by AAV Capsid ELISA

The viral particle titer was determined for each construct using AAV Titration ELISA kits designed for AAV9 and AAV2 (PROGEN™ Biotechnik GmbH, Heidelberg, Germany) according to the manufacturer's instructions. For AAV9, the mouse monoclonal ADK9 antibody was used for both the capture and detection steps. For AAVTT, the A20R monoclonal antibody was used for both capture and detection steps. Washes in the provided 1× Assay Buffer (ASSB) were performed between each step using a Molecular Devices™ (San Jose, CA, USA) AquaMax 4000 microplate washer. Samples were detected with a Molecular Devices™ SpectraMax M5e plate reader. Capsid titers were interpolated from the standard curve and are reported in Table 6.

TABLE 6

| | | | | Total | | |
|---|---|---|---|---|---|---|
| | | | Capsid | Viral | | Total |
| | | | Titer | Particles | Titer | Titer |
| Construct | Flask # | Capsid | (VP/mL) | (VP) | (VG/mL) | (VG) |
| CAG-hSLC6A1 | 1 | AAV9 | 4.43E+11 | 2.92E+13 | 7.37E+09 | 4.86E+11 |
| CAG-hSLC6A1 | 2 | AAV9 | 5.49E+11 | 3.62E+13 | 7.53E+09 | 4.97E+11 |
| CAG-hSLC6A1 | 1 | AAVtt | 2.82E+11 | 1.86E+13 | 1.11E+10 | 7.29E+11 |
| CAG-hSLC6A1 | 2 | AAVtt | 1.91E+11 | 1.26E+13 | 7.22E+09 | 4.77E+11 |

The viral genome titers obtained by ddPCR and capsid titers obtained by ELISA indicated that both AAV9 and AAVTT viral particles comprising a viral vector with a nucleic acid comprising a CAG promoter operably linked to a human SLC6A1 transgene could be successfully produced.

### Example 5: In-Vitro GABA Uptake of Mutated Forms of SLC6A1

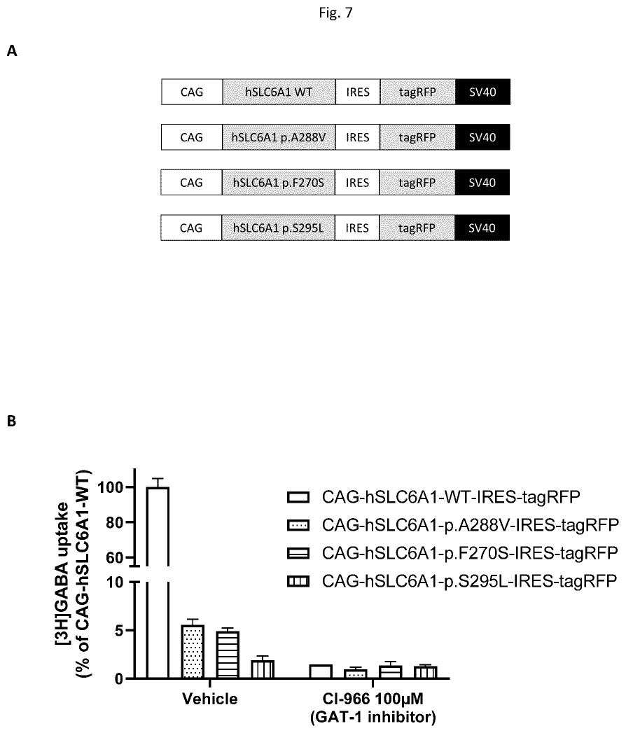

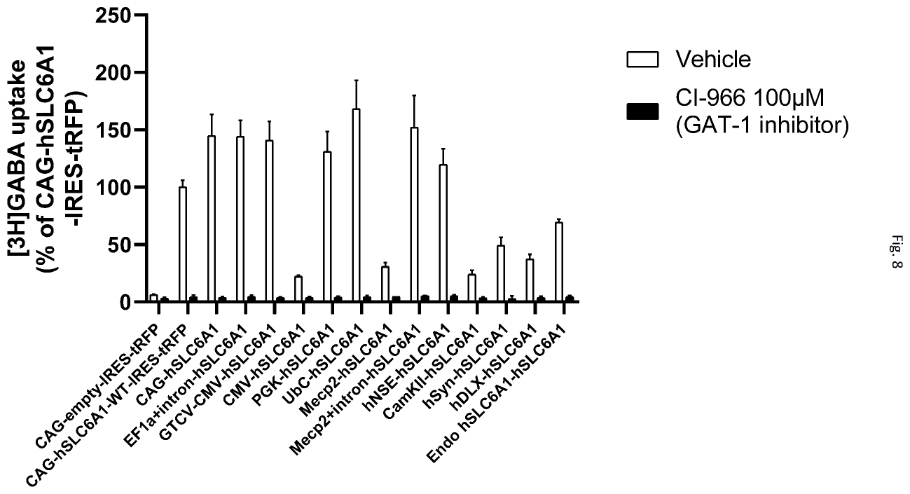

Different tool plasmids consisting of the CAG promoter expressing the hSLC6A1-WT sequence or described mutated forms of the later were produced (pathogenic variants: S295L, A288V, F270S, see also Example 3). All plasmids encoded a fluorescent protein (tagRFP) through an Internal Ribosome Entry Site (IRES) system allowing expression of 2 independent proteins (FIG. 7A). The latter allowed to confirm similar transfection levels between conditions.

COS7 cells (monkey fibroblast-like cell line) were seeded on scintillating microplates and were transfected with the tool plasmids described above using Lipofectamine 2000 and following manufacturer instructions. At 2 days post transfection, the level of transfection was checked with an epifluorescent microscope thanks to the tagRFP reporter gene. All transfection conditions were similar (data not shown). The COS7 cells were then submitted to a GABA uptake assay. Briefly, cells were washed and treated with a specific GAT-1 inhibitor (CI-966 (Tocris, Cat No 1296), final concentration of 100 μM in 1% DMSO) or with the vehicle alone (1% DMSO) for 10 min at 37° C. Cells were then treated with a mixture of tritiated and cold GABA ([3H]GABA 10 μCi/ml and 15 μM GABA final concentrations) for 10 min at 37° C. and the reaction was stopped using 1 mM cold GABA before quantification of the radioactivity with a Microbeta instrument (Perkin Elmer).

As illustrated in FIG. 7B, the described pathogenic variants of SLC6A1 showed significant decrease in the functional GABA uptake assay compared to wild type SLC6A1.

### Example 6: SLC6A1-Mediated GABA Uptake Under Different Promoters

SH-SY5Y cells (human neuroblastoma cell line) were transfected with the constructs as described in Example 1 using Lipofectamine 3000 and following manufacturer instructions. The positive control consisted of a plasmid encoding hSLC6A1 under the control of a CAG promoter expressed together with a tagRFP fluorescent protein whilst as negative control (and a matching plasmid lacking the hSLC6A1 sequence. At 2 days post transfection, SH-SY5Y cells were attested for either ICC analysis of GABA uptake assay. Briefly, ICC analysis was performed as follow: cells were fixed with 4% paraformaldehyde and stained with the primary antibodies rabbit monoclonal anti-GAT-1 (Ref: ab177483; Abcam™, Cambridge, MA, USA) at 1:250. Cells were then stained with goat anti-rabbit secondary antibodies conjugated to Alexa Fluor 488 at 1:1,000 prior to imaging. The level of transfection was estimated based on the number of fluorescent cells and is shown in Table 7. For the GABA uptake assay, cells were previously seeded on scintillating microplates. At 2 days post transfection, cells were washed and treated with a specific GAT-1 inhibitor (CI-966 (Tocris, Cat No 1296), final concentration of 100 μM in 0.8% DMSO) or with the vehicle alone (0.8% DMSO) for 10 min at 37° C. Cells were then treated with a mixture of tritiated and cold GABA ([3H]GABA 8 μCi/ml and 15 μM GABA final concentrations) for 10 min at 37° C. and the reaction was stopped using 1 mM cold GABA before quantification of the radioactivity with a Microbeta instrument (Perkin Elmer).

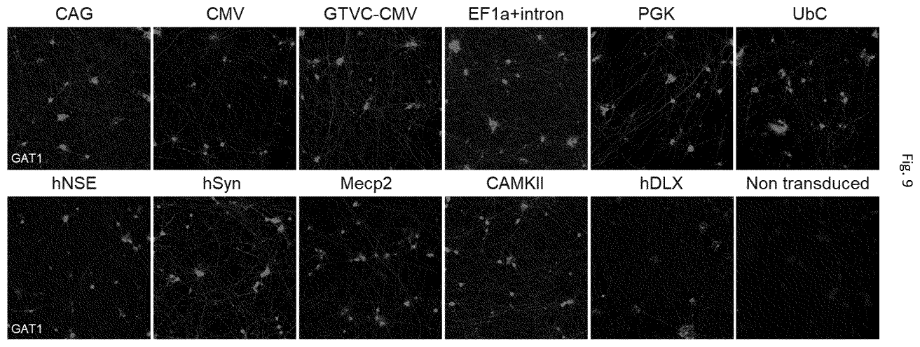

As shown in Table 7, the constructs were associated with different levels of pAAV transfection based on GAT-1 immunostaining. In addition, all promoters led to the expression of a functional GAT-1 protein, showed by an uptake of [3H]GABA that was present when cells were treated with the vehicle and inhibited when cells were treated with the GAT-1 inhibitor (FIG. 8).

TABLE 7

| CAG | | EF1a + | GTVC- | | | | | Mecp2 + | | | | | Endo |
|---|---|---|---|---|---|---|---|---|---|---|---|---|---|
| (tool) | CAG | intron | CMV | CMV | PGK | UBC | Mecp2 | intron | hNSE1 | CamKII | hSyn | hDLX | hSLC6A1 |
| +++ | +++ | +++ | +++ | ++ | ++ | +++ | + | ++ | + | + | ++ | + | ++ |

### Example 7: In Vitro Screening of Prom-hSLC6A1 Constructs in LVV Backbones

A gene edited iPSC-line carrying a DOX-inducible NGN2 expression was differentiated into iPSCs derived neurons (BIONi010-C-13 line). In this protocol, the NGN2 transcription factor is induced by doxycycline for 9 days to prime neuronal differentiation. At day in vitro (DIV) 21, the iPSCs derived NGN2 neurons were transduced with serial dilutions of Lentiviral vectors expressing hSLC6A1 under the control of different promoters of interest. At DIV 28, ICC analysis was performed as follow: cells were fixed with 2% paraformaldehyde and stained with the primary antibodies rabbit monoclonal anti-GAT-1 (Ref: ab177483; Abcam™, Cambridge, MA, USA) at 1:250. Cells were then stained with goat anti-rabbit secondary antibodies conjugated to Alexa Fluor 568 at 1:1000 prior to imaging. Imaging was performed with an InCell analyser 6000 instrument using empirical parameters. Representative images are shown in FIG. 9 with settings and parameters adapted to each image. Comparison of the signal to the non-transduced cells allowed the visualization of the hSLC6A1 transcription and expression under different promoters compared to endogenous GAT-1 in the iPSCs derived NGN2 neurons, a human based cell system which is not an immortalized cell line.

### Example 8: In Vitro and In Vivo Expression of Selected Cassettes

Four viral vectors were selected for further investigation and each was characterised by a different promoter. The CAG promoter (CAG), PGK promoter (PGK), hDLX promoter (hDLX) and the naturally occurring and endogenous SLC6A1 promoter (herein referred as ENDO) were used for driving human SLC6A1 expression. The constructs were engineered for the SLC6A1 protein to be expressed with a HA tag at the N-term. A viral vector consisting of hSyn-eGFP-NLS was used as a control (referred herein as “control AAV9”). The selected viral vectors were packaged in AAV9 and tested in vitro and in vivo. All in vivo experiments were conducted in compliance with guidelines issued by the ethics committee for animal experimentation according to Belgian law. The experiments were performed in accordance with the European Committee Council directive (2010/63/EU). All efforts were made to minimize animal suffering.

Mouse primary cortical neuronal cells were prepared from cortical tissue of E17 mouse embryos. Cortical tissues were dissociated using papain for 30 min at 37° C. and maintained in culture in Neurobasal™ Medium supplemented with B27 supplement 2%, GlutaMAX-I 1 mM and Penicillin-Streptomycin 50units/ml. Half medium change was performed every week. At division DIV 7, the neuronal cells were transduced with the different AAV9 vectors at 2 MOI (2.5E+6 GC/cell and 5.0E+5 GC/cell). The level of transduction was assessed with “control AAV9” and was high in both MOI conditions (MOI or multiplicity of infection is the ratio of agents e.g. virus, to infection targets e.g. cell). At DIV 13, cells were fixed and stained for different markers. Firstly, expression of the SLC6A1 transgene was demonstrated by measuring a positive anti-HA staining (1:100; Ref: 2367S, Cell Signaling Technology) that co-localized with the GAT-1 staining (1:200; Ref: ab177483; Abcam™, Cambridge, MA, USA). Co-location was observed in all viral vectors. Secondly, counter-staining was performed with an anti-MAP2 as a pan-neuronal marker (1:5000; Ref: ab5392; Abcam™, Cambridge, MA, USA), an anti-GABA (2.5 μg/mL; Ref: A2052; Sigma) to identify GABAergic neurons and an anti-GFAP (1:5000; Ref: ab7260; Abcam™, Cambridge, MA, USA) to identify astrocytes. The results (data not shown) confirmed that the hDLX promoter drives expression mainly in GABAergic neurons. In comparison, the PGK and ENDO promoters drove expression in astrocytes, and within the neuronal cell types, they drove expression at least in GABAergic neurons. The ENDO promoter showed better cell specificity for GABAergic neurons than the PGK promoter and also led to a pattern of expression more consistent with the endogenous expression of GAT-1 observed in non-transduced cells (wild type, without viral vectors, in control conditions). Expression through the CAG promoter led to strong expression and a MOI dependent negative effect on neuronal network development in vitro.

The in-vivo expression of the four selected viral vectors packaged in AAV9 was investigated by bilaterally injecting the viral vectors into the lateral ventricle in C57BL/6J male mice at postnatal day 1 as described in Table 8.

TABLE 8

| Age at | Delivery | | |
|---|---|---|---|
| treatment | Route | Viral Vector | Titer |
| Postnatal | ICV | AAV9-CAG-HA-hSLC6A1 | 1.32E+13 GC/mL |
| day 1 | bilaterally | AAV9-PGK-HA-hSLC6A1 | 1.33E+13 GC/mL |
| PND1 | injected | AAV9-hDLX-HA- | 1.27E+13 GC/mL |
| | 2 ul/ | hSLC6A1 | |
| | hemisphere | AAV9-ENDO-HA- | 1.23E+13 GC/mL |
| | | hSLC6A1 | |
| | | AAV9-hSYN-eGFP-NLS | 1.36E+13 GC/mL |
| | | (Control AAV9) | |
| | | Vehicle-PBS | |
| | | (Sterile Phosphate- | |
| | | buffered saline 1X) | |

Two additional groups of mice were injected with vehicle-PBS or “control AAV9” as controls.

During the 5 weeks after injection, in life assessment (clinical signs, adverse effects, body gain weight and mortality) was performed in all animal groups (vehicle-PBS, control AAV9, AAV9-CAG-HA-hSLC6A1, AAV9-PGK-HA-hSLC6A1, AAV9-hDLX-HA-hSLC6A1 and AAV9-ENDO-HA-hSLC6A1). Body weight differences were monitored once a week in order to assess the overall health status of the mice. There were no significant differences in the body gain weights in the different groups injected with the different viral vectors up and until the last evaluation. Mice injected with AAV9-CAG-HA-hSLC6A1 showed a decrease in survival (20% survival rate) over the course of the 5-week monitoring. Humane end points were reached, and mice were euthanized between the third and fourth week after injection. Mice injected with AAV9-PGK-HA-hSLC6A1 showed also a slightly decrease survival (85% survival rate) over the course of 5-week monitoring without displaying any clinical signs of toxicity. Regarding the other groups, none of the control mice injected with vehicle-PBS, control AAV9, AAV9-hDLX-HA-hSLC6A1 or AAV9-ENDO-HA-hSLC6A1 showed any signs of morbidity.

At 5 weeks post-injection, the animal was perfused with PBS under isoflurane anaesthesia, in accordance with European Committee Council directive (2010/63/EU). The brain was collected, dissected and submitted for biochemical analysis, i.e. DNA/RNA was extracted from left frontal cortex and hippocampus, while proteins were extracted from matching right frontal cortex. Briefly, DNA/RNA extraction was performed using the AllPrep mini kit (Qiagen™ 80204) following manufacturer instructions and including a DNAse treatment for the RNA extraction. The tissues were lysed in RLT Plus buffer (supplemented with beta-mercaptoethanol) using the Precellys 24 instrument (Bertin Technologies). The DNA concentration was measured and adjusted to 20 ng/μl for all samples. Then, 40 ng were submitted to qPCR using primers/probe specific for the SV40 polyA signal (present in all the AAV cassettes). The amount of mouse genomes was analysed using the ValidPrime® kit (tataabiocenter, A106P25). ValidPrime® is highly optimised and specific to a non-transcribed locus of gDNA that is present in exactly one copy per haploid normal genome. For both SV40p and ValidPrime®, absolute copy numbers were determined using the standard curve method. Reverse transcription (RT) PCR of 500 ng of RNA was performed using the kit High Capacity cDNA RT Kit+RNase Inhibitor (Applied Biosystems cat n° 4374966). Subsequently, the obtained cDNAs were submitted to the SV40 polyA signal qPCR, as well as two reference genes for normalization of the results. Relative expression was determined and scaled to the average value for all groups. For the protein extraction, tissues were lysed in RIPA buffer (Pierce, 89900) including 2× concentrated Protease and phosphatase inhibitors cocktail (Cell Signaling Technology, #5872) using the Precellys 24 instrument (Bertin Technologies) and cooling system. The samples were left on ice for 30 min, centrifuged and the supernatant was collected as the final protein extract. Protein concentration were determined using the BCA Protein Assay Kit (Pierce, 23227) and 10 μg of protein were mixed with Laemli buffer and beta-mercaptoethanol and incubated at 30° C. for 20 minutes prior to SDS-Page. Gels were transferred to nitrocellulose membranes and then submitted to standard WB procedure. Briefly, membranes were incubated in blocking solution (Ref: 927-50000; Li-Cor) for 1 hour at 4° C. The primary antibodies consisted of rabbit monoclonal anti-GAT-1 (1:2000; Ref: ab177483; Abcam™, Cambridge, MA, USA), mouse monoclonal anti-HA (1:1000; Ref: 2367S, Cell Signaling Technology) and mouse monoclonal anti GAPDH (1:10000; Ref: G8795, Sigma). The secondary antibodies used were IRDye® 680RD Donkey anti-Mouse IgG Secondary Antibody (1:20000; Ref: 926-68072, Li-Cor) and IRDye® 800CW Donkey anti-Rabbit IgG Secondary Antibody (1:20000; Ref: 926-32213, Li-Cor).

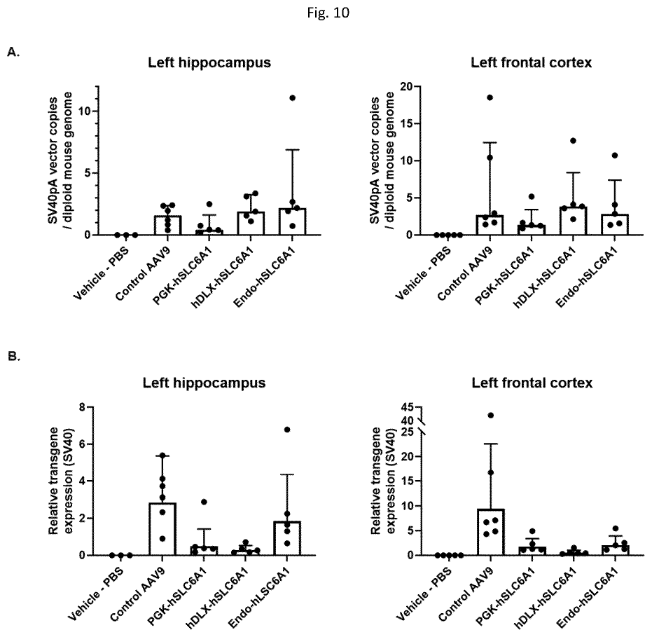

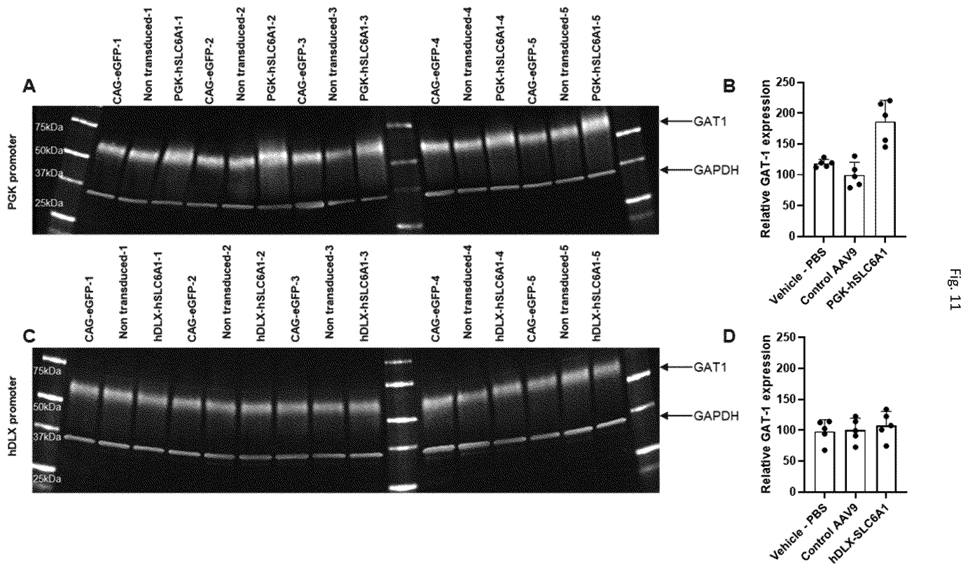

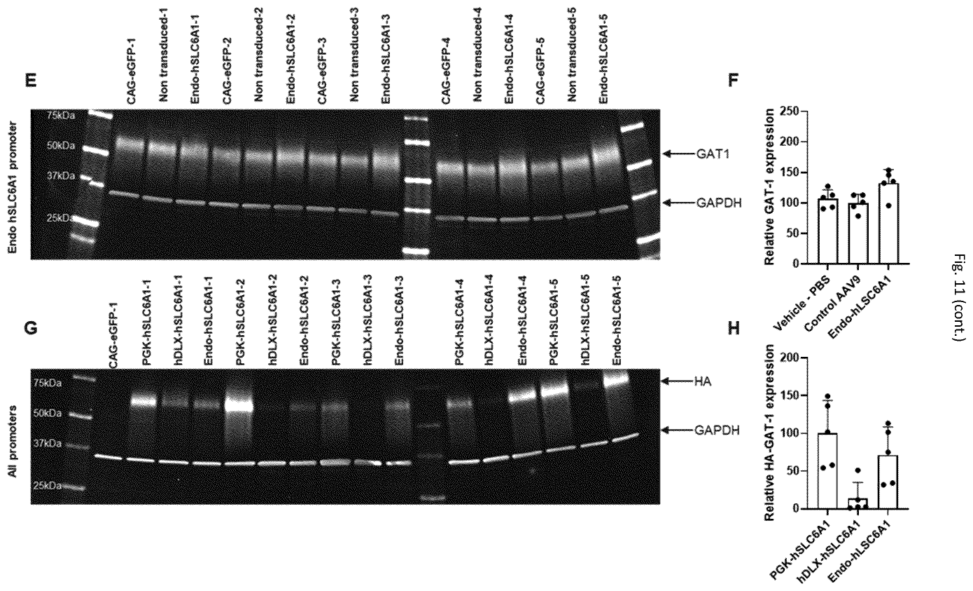

As illustrated in FIG. 10, panel A, significant viral genome copies per diploid mouse genomes were detected in the DNA extract demonstrated an efficient and homogenous AAV9 transduction among the different viral vectors. The viral vector comprising the PGK promoter had a slightly reduced transduction level. RNA expression analysis revealed expression of the transgene in all viral vectors analysed (FIG. 10, panel B). Relative comparison allowed general ranking of promoter strength among viral vectors for SV40pA mRNA expression. The control AAV9 construct led to high level of expression compared to the viral vectors with the SLC6A1 transgene. Among them the viral vectors comprising the PGK and ENDO promoters (with the latter one being more expressed in the hippocampus) showed higher expression than the hDLX promoter.

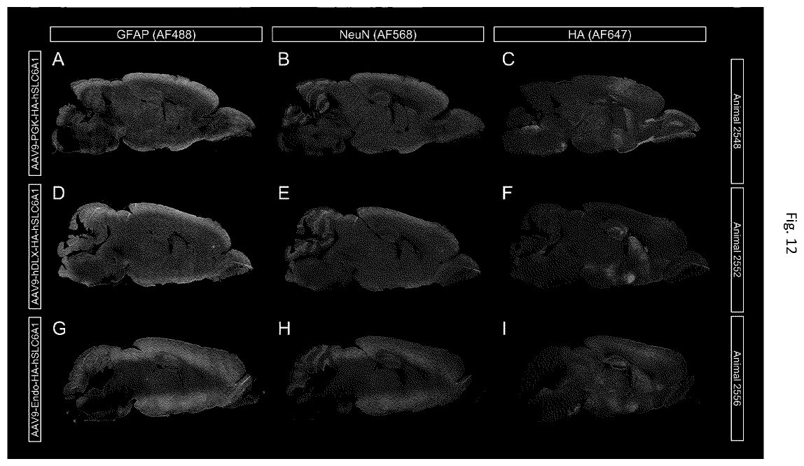

Protein analysis confirmed the DNA and RNA results, with a marked overexpression of GAT-1 at the tissue level for both the viral vectors comprising the PGK and ENDO promoters compared to the control groups (non-transduced animals injected with vehicle-PBS or transduced animals injected with control AAV9) (FIG. 11). Promoters PGK and ENDO led to similar levels of GAT-1 protein expression, whilst hDLX promoter showed lower, but yet detectable, expression.

Brain samples from additional mice injected with AAV9-PGK-HA-hSLC6A1, AAV9-hDLX-HA-hSLC6A1 and AAV9-ENDO-HA-hSLC6A1 were analysed by immunohistochemistry. Fresh frozen sections (12 μm thickness; sagittal) were generated with a cryostat-microtome by QPS Austria (Austria) and stored at −80° C. All of the following incubation steps were carried out at room temperature. Triple immunofluorescence labelling was performed on mouse brain sections using the following protocol: sections were incubated with NeuN (1:2,000; Abcam, ab177487), GFAP (1:2,000; SySy, 173006) and biotin-conjugated HA (1:5,000; Biolegend, 901505) primary antibodies together, diluted in PBS containing 0.3% Triton X-100, overnight in a humidified chamber. Following incubation, the sections were washed 3 times with PBS, then incubated with the anti-chicken Alexa Fluor 488 and anti-rabbit Alexa Fluor 546 secondary antibodies and streptavidin-conjugated Alexa Fluor 647 (all diluted at 1:1,000 in PBS; all from Thermo Fisher) for 1 hour. Then, they were counterstained with DAPI to label cell nuclei and washed 3 times with PBS. The sections were finally mounted with Prolong Gold antifade mounting media (Life Technologies) and a coverslip was applied. Digital images of stained sections were obtained using an AxioScan Z1 slide scanner with a 20× objective (Zeiss). The immunolabeling to HA was used to study the distribution of human SLC6A1 overexpressed from the different promoters, including PGK, human DLX and SLC6A1 endogenous promoter.

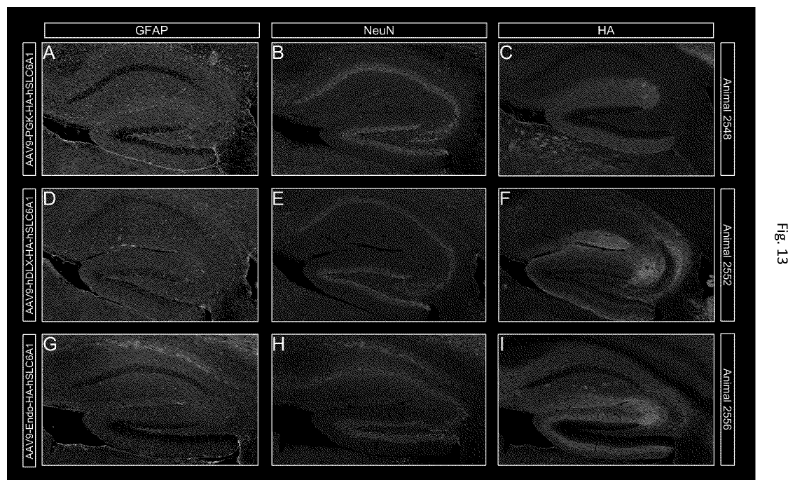

GAT-1 protein expression detected through the HA-tag labelling under the effect of the 3 different promoters was detected throughout the brain, mainly in the striatum, hippocampus, cerebral cortex, hypothalamus, pallidum and septum (FIG. 12, panels C, F and I). For the PGK promoter, the HA-tag labelling was also observed in the medulla and cerebral nuclei (FIG. 12, panel C).

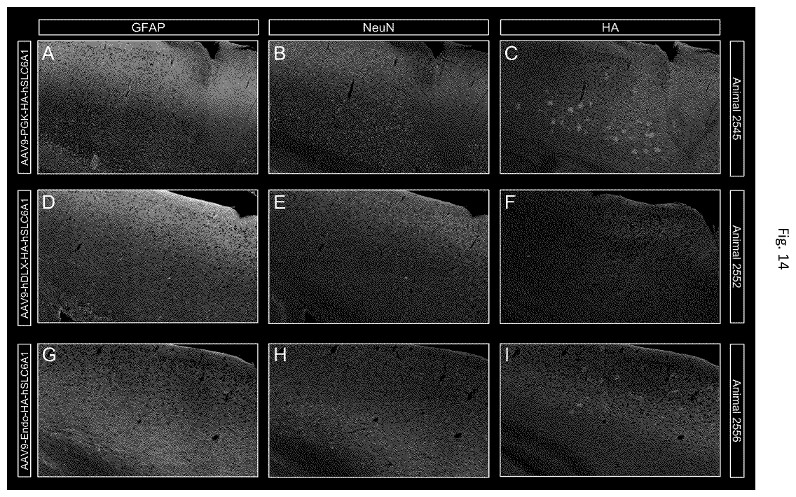

GAT-1 expression was also observed in the hippocampus with slightly distinct patterns according to the promoter. With all 3 promoters, HA-tag staining was observed in neuronal projections composing the molecular layer of the dentate gyrus and hippocampus and stratum oriens (FIG. 13, panels C, F and I). In addition, the hDLX promoter led to the expression of GAT-1 in the Cornus ammonis 3 (CA3) (FIG. 13), while the PGK and ENDO promoters led to the expression of GAT-1 in astrocytes which were GFAP+ (FIG. 13, panels C and I).

GAT-1 expression was observed in the neuropil of the cerebral cortex (FIG. 14, panels C, F and I). Specifically, PGK and ENDO promoters led to the expression of GAT-1 in astrocytes which were also labeled with GFAP.

A pathological safety assessment of selected tissues was also carried out.

Following the in vivo phase of the study, the brain was split longitudinally into two hemispheres and one hemisphere was used for pathological examination. The hemi-brain together with the spinal cord, dorsal root ganglia, liver, kidney, spleen, thymus and eyes were fixed in 10% neutral buffered formalin, embedded in paraffin, processed to wax blocks, sectioned at approximately 5 uM thickness and stained with Hematoxylin and Eosin (H&E).

A series of tissues (brain (7 transverse sections (Bolon et al. Toxicol Pathol 2018 June; 46(4):372-402. doi: 10.1177/0192623318772484), spinal cord with dorsal root ganglia (6 transverse or longitudinal sections; cervical, thoracic, and lumbar), liver (2 sections; left and caudate lobes), kidney (2 sections; left and right organs), spleen (1 section), thymus (1 section) and eyes (2 sections, left and right organs)), from n=28 mice were evaluated by light microscopy.

No findings specific to the treatment arm were identified within the brain, spinal cord/dorsal root ganglia, kidneys, or eyes (pigmentation of the retinal pigmented epithelium in these wild-type mice precluded an assessment of lipofuscin/pigment content).

Several changes were noted in the brain that were considered the result of mechanical (procedural) damage at necropsy or from the injection procedure characterised by dark neuron artefacts, on occasion accompanied by architectural disruption.

Within the liver, a number of animals administered control AAV9 had minimal, diffuse, hepatocyte vacuolation, predominantly within the midzonal regions which was also observed in individual animals administered with viral vectors AAV9-CAG-HA-hSLC6A1, AAV9-PGK-HA-hSLC6A1, or AAV9-hDLX-HA-hSLC6A1.

Increased mitotic figures (up to slight severity) were also noted within the liver of some animals administered with viral vectors AAV9-CAG-HA-hSLC6A1 or AAV9-PGK-HA-hSLC6A1 (and occasionally in the kidneys—not recorded).

Finally, two animals from treatment groups injected with viral vectors AAV9-CAG-HA-hSLC6A1 and AAV9-PGK-HA-hSLC6A1 also presented minimal hepatocyte single cell necrosis.

Other findings, such as minimal inflammatory cell infiltration, congestion, and focal necrosis, were considered to lie within a spectrum of expected normal background variation and were not considered to be related to the viral vectors. Animals administered AAV9-ENDO-HA-hSLC6A1 had liver morphology consistent with a normal background range.

Within the spleen, individual animals administered with viral vectors AAV9-CAG-HA-hSLC6A1, AAV9-PGK-HA-hSLC6A1 or AAV9-ENDO-HA-hSLC6A1 had minimal-to-slight levels of extra-medullary hematopoiesis. This was considered likely to reflect a test article-related reduction in the expected hematopoietic cellularity within this tissue (recorded as moderate in control animals).

### Example 9: In Vivo Evaluation of Selected Viral Vectors in Transgenic SLC6A1 Disease Mouse Model

To evaluate the efficacy of selected viral vectors, a transgenic mouse model that recapitulates human SLC6A1 haploinsufficiency-mediated epilepsy was generated. The model used was a knock-in (KI) mouse model on a C57BL/6J background bearing the S295L point mutation in the SLC6A1 gene (SLC6A1^{+/S295L}) generated at Shanghai Model Organisms. The S295L mutation had been functionally validated in vitro, leading to complete loss-of-function of GAT-1. The mutation is believed to occur in a region that has been shown to harbor pathogenic mutations and was found in a patient with absence seizures and developmental delay (https://slc6a1connect.org/). All in vivo experiments were conducted in compliance with guidelines issued by the ethics committee for animal experimentation according to Belgian law. The experiments were performed in accordance with the European Committee Council directive (2010/63/EU). All efforts were made to minimize animal suffering.

Heterozygous KI (SLC6A1^{+/S295L}) and wildtype littermate (SLC6A1^{+/+}) male mice were bilaterally injected into lateral ventricle with one of 3 viral vectors (AAV9-PGK-HA-hSLC6A1, AAV9-hDLX-HA-hSLC6A1 and AAV9-ENDO-HA-hSLC6A1) at postnatal day 1 as described in Table 9.

TABLE 9

| | Age at | Delivery | Viral | | Sample | In Life | Seizure |
|---|---|---|---|---|---|---|---|
| Genotype | treatment | Route | Vector | Titer | size | Assessment | monitoring |
| HET | Postnatal | ICV | AAV9- | 1.33E+13 | 10 | Clinical | YES |
| (KI) | day 1 | bilaterally | PGK-HA- | GC/mL | | signs, | In vivo |
| | PND1 | injected | hSLC6A1 | | | adverse | wireless EEG |
| | | 2 ul/ | AAV9- | 1.27E+13 | 11 | effects, body | video- |
| | | hemisphere | hDLX- | GC/mL | | gain weight | telemetry |
| | | | HA- | | | and | recordings |
| | | | hSLC6A1 | | | mortality | (6-7 weeks |
| | | | AAV9- | 1.23E+13 | 15 | during 7 | post- |
| | | | ENDO- | GC/mL | | weeks post- | injection) |
| | | | HA- | | | injection | |
| | | | hSLC6A1 | | | | |
| | | | Vehicle- | — | 11 | | |
| | | | PBS | | | | |
| | | | (Sterile | | | | |
| | | | Phosphate- | | | | |
| | | | buffered | | | | |
| | | | saline | | | | |
| | | | 1X) | | | | |
| WT | Postnatal | ICV | Vehicle- | — | 10 | | NO |
| | day 1 | bilaterally | PBS | | | | |
| | PND1 | injected | (Sterile | | | | |
| | | 2 ul/ | Phosphate- | | | | |
| | | hemisphere | buffered | | | | |
| | | | saline | | | | |
| | | | 1X) | | | | |

One additional group of mice from each genotype were injected with vehicle-PBS to be used as control. Clinical signs were monitored once a week over the course of the 3 weeks post-injection and daily from week 3 to 7 post-injection in order to assess the overall health status of the mice. Terminal assessment of the brain, plasma and organs collection by biochemical analysis, histopathology, immunohisto-chemistry, and transgene expression was performed at 7 weeks post-injection.

There were no significant differences in the body gain weights in the different groups injected with the different viral vectors up and until the last evaluation. No mortality was observed during the follow-up period (week 3-7 post injection).

Six weeks after injections, in vivo wireless EEG (electroencephalogram) video-telemetry recordings were performed for 1 week to evaluate seizure occurrence. SLC6A1^{+/S295L} mice were surgically implanted with subcutaneous telemetry transmitter and cortical EEG electrodes 5 weeks after injections. Surgery was performed under sterile/aseptic conditions. Anaesthetized mice (Isoflurane in oxygen—Induction: 5% at 2 l/min, maintenance 2.5-1.5% at 1.5 l/min) were placed in a stereotaxic frame with heating pad, holes were drilled on the skull surface of the prefrontal cortex (over bregma) for the recording electrode and on the skull surface of the cerebellum (behind the lambda) for the reference electrode. Thereafter, an Open Source Instruments (OSI) A3028S2 ECoG transmitter was implanted subcutaneously over the dorsum with the attached wires extending subcutaneously up to the cranium where the recording and reference electrodes were positioned through each hole approximately 0.5 mm into the brain parenchyma. Each electrode was secured in place with a screw (Plastics One). The whole assembly was held in place with cyanoacrylate and dental cement forming a small, circular headpiece and the dorsum was closed with nylon absorbable suture material. Post-operative medication and pain management included a second Carprofen dose (10 mg/kg) 24 hours following the pre-surgery dose. After the surgery, mice were recovering in warm-chamber for 2-3 h. For in vivo wireless EEG video-telemetry recordings, mice were group housed (2-3 mice/cage). Mice cages were placed in Faraday enclosures to facilitate recordings. Welfare monitoring of implanted mice was conducted once per day for 2 weeks. Mice were weighed daily for 4 days, thereafter weekly. All recordings were carried in a purposely designed recording room with temperature and humidity control in order to decrease ambient interference and improve the reception of the transmitting signals. Signals were radio transmitted from the implanted transmitter to the antennas placed inside the Faraday enclosures. EEG signal from one recording channel was digitized at 256 Hz (Band-pass filter: 0.3-80 Hz). Spike wave discharges (SWDs), typical of absence seizures, were analysed with an in-house automated seizure detection software. SWDs detection algorithm was based on event duration analysis (>2 s), band frequency analysis (5-9 Hz) and identification of specific fundamental harmonic frequencies. Each SWD detected by the algorithm was confirmed by at least one experienced observer in a blinded fashion. A period of high SWD occurrence (5 hours from 1 pm to 6 pm), was initially observed in the transgenic line SLC6A1^{+/S295L} non-injected with the viral vectors. Consequently, EEG analysis was performed during this period for the different viral vector and control groups. A total of 4 animals were excluded from the analysis due to the occurrence of technical artefacts in the EEG signal in the following groups: AAV9-PGK-HA-hSLC6A1 (2 out of 10) and AAV9-ENDO-HA-hSLC6A1 (2 out of 15). An additional 2 animals were also removed from the analysis in the group AAV9-hDLX-HA-hSLC6A1 (2 out of 11); one displayed artefact in the EEG and the other one was not transduced (no detection of viral genome copies in brain tissue, as mentioned below). The difference between groups was analysed by non-parametric one-way ANOVA (Kruskal-Wallis test) followed by a Dunn's post hoc multiple comparisons test (**p<0.01).

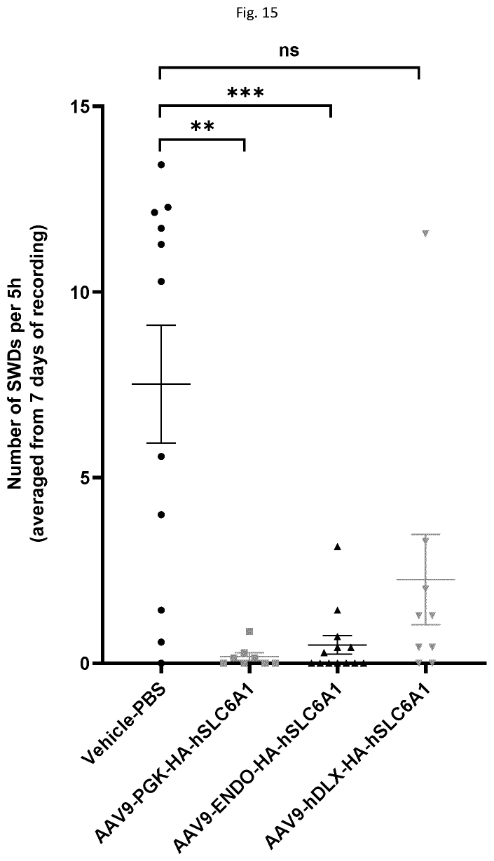

As illustrated in FIG. 15, the average number of SWDs per day recorded over 7 consecutive days during the peak hours of SWD occurrence was significantly reduced by 97% and 93% in SLC6A1^{+/S295L} mice injected with either AAV9-PGK-HA-hSLC6A1 or AAV9-ENDO-HA-hSLC6A1, respectively, compared to the control group. The reduction in number of SWDs in SLC6A1^{+/S295L} mice injected with AAV9-hDLX-HA-hSLC6A1 did not reach in this experiment statistical significance compared to the control group.

Furthermore, biochemical analysis was performed on the brain tissues from the animals injected with the different viral vectors. Animal were sacrificed 7 weeks post injection following the same methodology as described in Example 8. Caudal cortex was collected and subjected to DNA/RNA extraction and matching half medial frontal cortex was used for protein extraction using the same methodology described in Example 8.

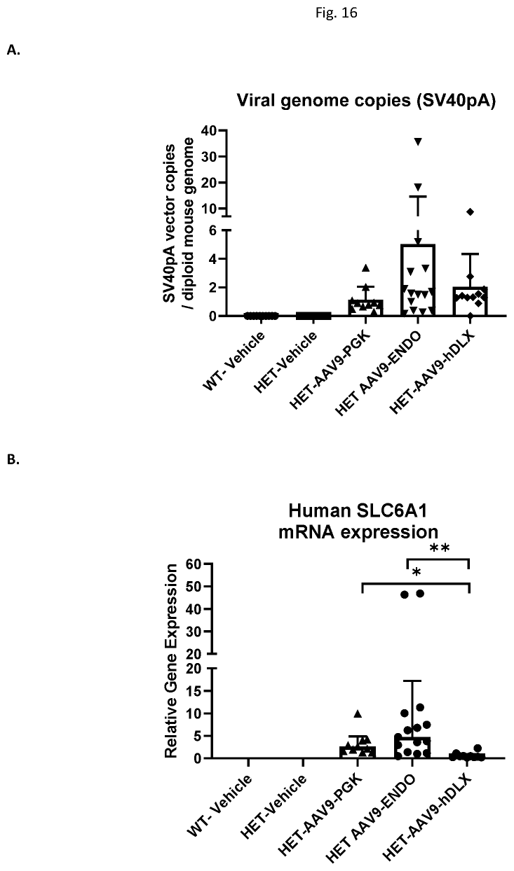

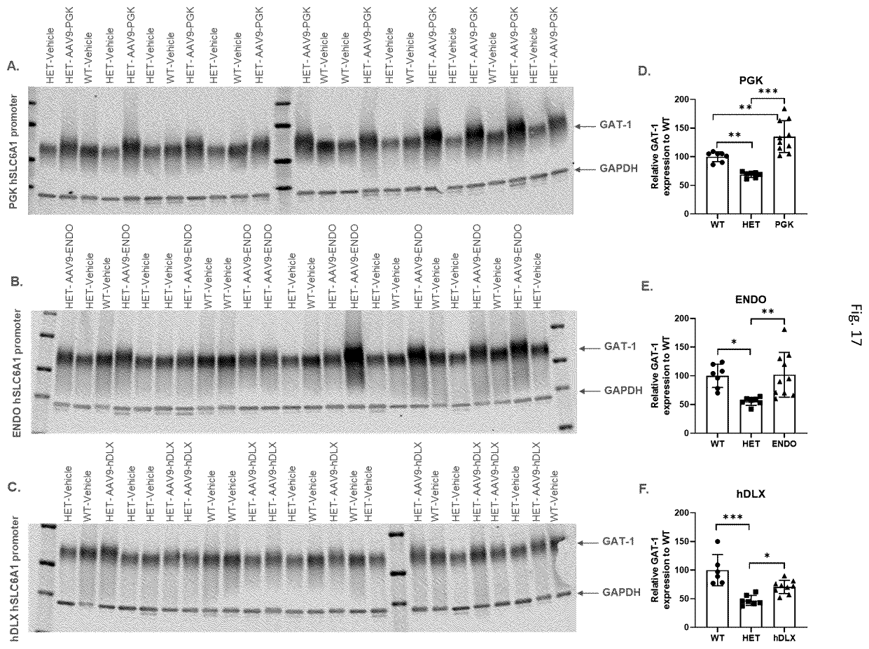

As illustrated in FIG. 16A, significant viral genome copies per diploid mouse genomes were detected in the DNA extracts demonstrating an efficient and homogenous AAV9 transduction among the different viral vectors used (with the exception of one animal in the AAV9-hDLX group that showed no viral transduction). FIG. 16B shows mRNA expression in all AAV9 transduced groups. No significant difference was observed between the PGK and ENDO promoters for SLC6A1 expression. On the other hand, the hDLX promoter showed significant reduced mRNA expression compared to the other groups.

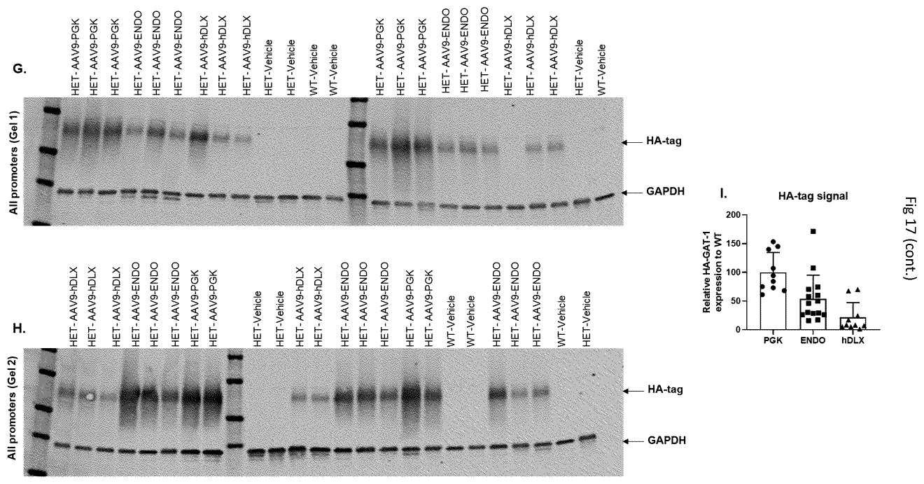

The protein analysis confirmed as expected significant reduction of GAT-1 expression in the SLC6A1^{+/S295L} mice (referred as HET in the figures) compared to their WT littermates (FIG. 17 panels D, E and F). As illustrated by FIG. 17, the western blot gels and the graphs show that GAT-1 expression was significantly increased upon AAV9 injection in the SLC6A1^{+/S295L} mice compared to the vehicle injected SLC6A1^{+/S295L} mice (referred as HET in the figures). Overexpression of GAT-1 was observed for all viral vectors used. The PGK promoter increased the expression over wild-type (WT) levels while the ENDO promoter showed similar expression levels to WT rescuing the haploinsufficiency. The hDLX promoter showed as well increased expression over the SLC6A1^{+/S295L} mice. Similarly to the observations in example 8 in WT animals when looking at the HA signal the promoter's strength could be compared. As observed before PGK promoter showed the strongest protein expression followed by ENDO and the hDLX promoter in the SLC6A1^{+/S295L} mice.

1. A nucleic acid construct comprising a transgene encoding:
i. a gamma butyric acid (GABA) transporter protein 1 (GAT-1) comprising SEQ ID NO: 18, 19, 20; or
ii. a sequence having at least 95% sequence identity to SEQ ID NO: 18, 19 or 20 and retaining functionality as GAT-1; or
iii. a naturally-occurring GAT-1 variant comprising, with reference to SEQ ID NO: 18, one or more mutations.

2. The nucleic acid construct according to claim 1, wherein the transgene is a solute carrier family 6 member 1 (SLC6A1) gene, wherein the transgene comprises:
i. SEQ ID NO: 15, 26, 27, 28 or 29, or
ii. a sequence having at least 95% sequence identity to SEQ ID NO: 15, 26, 27, 28 or 29.

3. The nucleic acid construct according to claim 1, further comprising a promoter operably linked to said transgene, wherein said promoter comprises:
a. SEQ ID NO: 1; or
b. SEQ ID NO: 3; or
c. SEQ ID NO: 4; or
d. SEQ ID NO: 5 or SEQ ID NO: 35 or SEQ ID NO: 6; or
e. SEQ ID NO: 7; or
f. SEQ ID NO: 8; or
g. SEQ ID NO: 9; or
h. SEQ ID NO: 10; or
i. SEQ ID NO: 11, or SEQ ID NO: 11 operably linked in a 5′ to 3′ orientation to SEQ ID NO: 12; or
j. SEQ ID NO: 14.

4. The nucleic acid construct according to claim 1, wherein the construct comprises a polyadenylation signal sequence comprising SEQ ID NO: 17.

5. A viral vector comprising the nucleic acid construct according to claim 1, wherein the viral vector further comprises inverted terminal repeat (ITR) at 5′ and/or 3′ flanking said nucleic acid construct.

6. The viral vector according to claim 5, wherein the 5′ITR and/or the 3′ITR comprises a natural adeno-associated virus (AAV) ITR.

7. The viral vector according to claim 5, wherein the 5′ITR comprises SEQ ID NO: 22 and/or the 3′ITR comprises SEQ ID NO: 23.

8. A viral particle comprising a nucleic acid construct according to claim 1.

9. The viral particle according to claim 8, wherein the viral particle comprises at least a VP1 capsid protein from AAV2, AAV5, AAV6, AAV8, AAV9, AAV10, AAV-true type (AAVtt) or combinations thereof.

10. The viral particle according to claim 9, wherein the capsid protein is from AAVtt and comprises SEQ ID NO: 24 or a sequence at least 98.5% identical to SEQ ID NO: 24.

11. A viral vector comprising a nucleic acid construct comprising a transgene encoding:
i. a gamma butyric acid (GABA) transporter protein 1 (GAT-1) comprising SEQ ID NO: 18, 19, 20; or
ii. a sequence having at least 95% sequence identity to SEQ ID NO: 18, 19 or 20 and retaining functionality as GAT-1; or
iii. a naturally-occurring GAT-1 variant comprising, with reference to SEQ ID NO: 18, one or more mutations;
wherein said viral vector further comprises a promoter operably-linked to said transgene; wherein the nucleic acid construct comprised in said viral vector comprises a polyadenylation signal sequence; and wherein said viral vector further comprises inverted terminal repeat (ITR) at 5′ and/or 3′ flanking said nucleic acid construct.

12. A viral vector comprising a nucleic acid construct comprising a transgene which is a solute carrier family 6 member 1 (SLC6A1) gene, wherein the transgene comprises:
i. SEQ ID NO: 15, 26, 27, 28 or 29, or
ii. a sequence having at least 95% sequence identity to SEQ ID NO: 15, 26, 27, 28 or 29,
wherein said viral vector further comprises a promoter operably linked to said transgene; wherein the nucleic acid construct comprised in said viral vector comprises a polyadenylation signal sequence and wherein said viral vector further comprises inverted terminal repeat (ITR) at 5′ and/or 3′ flanking said nucleic acid construct.

13. The viral vector according to claim 12, wherein said transgene encodes a gamma butyric acid (GABA) transporter protein 1 (GAT-1) comprising SEQ ID NO: 18.

14. The viral vector according to claim 11, wherein the polyadenylation signal sequence comprises SEQ ID NO: 17.

15. A viral particle comprising the viral vector according to claim 11.

16. The viral particle according to claim 15, wherein the viral particle comprises at least a VP1 capsid protein from an AAV2, AAV5, AAV6, AAV8, AAV9, AAV10, AAV-true type (AAVtt) or combinations thereof.

17. The viral particle according to claim 16, wherein the capsid protein is from AAV9 and SEQ ID NO: 25 or from AAVtt and comprises SEQ ID NO: 24.

18. A plasmid comprising the nucleic acid construct according to claim 1, wherein the transgene comprises the nucleic acid sequence of SEQ ID NO: 15 operably linked to a promoter comprising the nucleic acid sequence of SEQ ID NO: 14.

19. A host cell for producing a viral particle comprising a viral vector according to claim 8.

20. A host cell comprising:
a. a nucleic acid construct according to claim 4; and
b. a nucleic acid construct encoding AAV rep and/or cap genes which does not carry the ITR sequences.

21. A method of producing a viral particle, the method comprising the step of:
a. culturing a host cell according to claim 19 in a culture medium; and
b. harvesting the viral particles from the host cell culture media and/or inside the host cells.

22. A pharmaceutical composition comprising a viral particle according to claim 8 in combination with one or more pharmaceutical acceptable excipient, diluent or carrier.

23.-27. (canceled)

28. A method of treating and/or preventing a disease characterised by SLC6A1 haploinsufficiency, the method comprising administering to a subject in need thereof viral particles according to claim 8.

29. The method according to claim 28, wherein the method is for results in restoration of GAT-1 function and/or decreased seizure frequency.

30. The method according to claim 28, wherein said disease is associated with at least one mutation in a patient which leads to a pathological GAT-1 variant, wherein said pathological GAT-1 variants comprises a mutation or combinations of mutations.

31. The method according to claim 30 wherein said mutation comprises, with reference to SEQ ID NO: 18, R44W, R44Q, R50L, D52E, D52V, F53S, S56F, G63S, N66D, G75R, G79R, G79V, F92S, G94E, G105S, Q106R, G112V, Y140C, C173Y, G232V, F270S, R277H, A288V, S295L, G297R, A305T, G307R, V323I, A334P, A367T, V342M, A357V, G362R, L366V, F385L, G393S, S456R, S459R, M487T, V511L, G550R or combinations thereof.

32. The nucleic acid construct of claim 1, wherein the naturally-occurring variant comprises, with reference to SEQ ID NO: 18, one or more mutations selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; and Val578Ile.

33. The nucleic acid construct of claim 2, wherein the transgene comprises SEQ ID NO: 15.

34. The nucleic acid construct of claim 3, wherein the promoter comprises
a. SEQ ID NO: 1 operably linked in a 5′ to 3′ orientation to SEQ ID NO: 2; or
b. SEQ ID NO: 35 operably linked in a 5′ to 3′ orientation to SEQ ID NO: 6; or
c. SEQ ID NO: 7 operably linked in a 5′ to 3′ orientation to SEQ ID NO: 34; or
d. SEQ ID NO: 11 operably linked in a 5′ to 3′ orientation to SEQ ID NO: 12,
wherein SEQ ID NO: 12 is operably linked in a 5′ to 3′ orientation to SEQ ID NO: 13.

35. A viral particle comprising a viral vector according to claim 5.

36. The viral vector of claim 11, wherein (iii) the one or more mutations of the naturally-occurring variant are selected from the group consisting of Ala2Thr; Asp165Tyr; Arg277Ser; Ile434Met; Arg579His; Gly5Ser; Arg172Cys; Arg277Cys; Ser470Cys; Pro580Ser; Asp10Asn; Arg172His; Arg277Pro; Ile471Val; Pro587Ala; Gly11Arg; Phe174Tyr; Ser280Cys; Gly476Ser; Ala589Val; Ile13Thr; Ser178Asn; Asn310Ser; Arg479Gln; Ile599Val; Glu16Lys; Asn181Asp; Tyr317His; Lys497Asn; Glu19Gly; Asn181Lys; Ile321Val; Phe502Tyr; Pro21Thr; Arg195His; Ser328Leu; Ile506Val; Lys33Glu; Met197Leu; Met332Val; Ala509Val; Val34Leu; Asp202Glu; Val337Ile; Thr520Met; Asp40Asn; Lys206Glu; His347Arg; Gly535Val; deletion of Met1; stop codon after Glu411; Asp43Glu; Arg211Cys; Ala354Val; Leu547Phe; Lys76Asn; Ile220Val; Leu375Met; Met552Ile; Asn77Asp; Ile220Asn; Ile377Val; Met555Val; Ile84Phe; Ala221Thr; Ile405Val; Thr558Asn; Phe87Leu; Val240Ala; Val409Met; Arg566His; Ile91Val; Phe242Val; Leu415Ile; Gln572Arg; Val142Ile; Tyr246Cys; Arg417Cys; Pro573Thr; Thr156Asn; Arg257Cys; Arg417His; Pro573Ser; Thr158Pro; Arg257His; Arg419Cys; Ser574Asn; Asp165Asn; Thr260Met; Arg419His; and Val578Ile.

37. The viral vector of claim 11, wherein the promoter operably-linked to the transgene comprises the sequence of SEQ ID NO: 4.

38. The viral vector of claim 12, wherein the promoter operably-linked to the transgene comprises the sequence of SEQ ID NO: 4.

39. The method of claim 23, wherein the disease preferably comprises single-gene epilepsies accompanied by cognitive, motor-behavioural comorbidities, early onset developmental and epileptic encephalopathy, epileptic encephalopathy, child onset Epilepsy Syndromes, myoclonic atonic epilepsy (MAE), MEA-like and other epilepsy indications such as Lennox-Gastaut Syndrome as well as autism spectrum disorder and schizophrenia or diseases associated with impaired GABA uptake or combinations thereof, with or without autism and/or schizophrenia.

40. The host cell of claim 20 further comprising:
c. a nucleic acid construct comprising viral helper genes.

41. A host cell comprising:
a. the viral vector according to claim 12, and
b. a nucleic acid construct encoding AAV rep and/or cap genes which does not carry the ITR sequences.

42. The host cell of claim 41 further comprising:
c. a nucleic acid construct comprising viral helper genes.

43. The method of claim 21, wherein the host cell comprises a nucleic acid construct according claim 4 and a nucleic acid construct, encoding AAV rep and/or cap genes which does not carry the ITR sequences.

44. The method of claim 21, wherein the host cell comprises the viral vector according to claim 12 and a nucleic acid construct, encoding AAV rep and/or cap genes which does not carry the ITR sequences.

45. A method for restoring GAT-1 function and/or decreasing seizure frequency, the method comprising administering to a subject in need thereof viral particles according to claim 8.
